(19) United States
(12) Patent Application Publication
(10) Pub. No.: US 2006/0264431 A1
(43) Pub. Date: Nov. 23, 2006

(54) P38 inhibitors and methods of use thereof

(75) Inventors: Mark Munson, Louisville, CO (US); James Rizzi, Longmont, CO (US); Ganghyeok Kim, Superior, CO (US)

Correspondence Address: HOGAN & HARTSON LLP, ONE TABOR CENTER, 1200 17TH STREET, SUITE 1500, DENVER, CO, 80202, US

(73) Assignee: ARRAY BIOPHARMA INC.

(21) Appl. No.: 11/431,723

(22) Filed: May 10, 2006

Related U.S. Application Data
(60) Provisional application No. 60/679,883, filed on May 11, 2005.

(51) Int. Cl. A61K 31/5377; A61K 31/454; A61K 31/416; C07D 413/02; C07D 403/02

(52) U.S. Cl. 514/232.5; 514/320; 514/406; 546/199; 544/140; 548/361.1

(57) ABSTRACT

Inhibitors of p38 and methods for producing these inhibitors are provided. Also provided are pharmaceutical compositions comprising the inhibitors of the invention, methods of utilizing the inhibitors and pharmaceutical compositions comprising said inhibitors in the treatment and prevention of various disorders mediated by p38, and kits comprising said inhibitors.

### CROSS-REFERENCE TO RELATED APPLICATIONS

This application claims priority to U.S. Provisional Application Ser. No. 60/679,883, filed May 11, 2005, which is incorporated herein by reference in its entirety.

### BACKGROUND OF THE INVENTION

1. Field of the Invention

This invention relates to compounds which are inhibitors of p38 MAP kinase and related kinases, pharmaceutical compositions containing the compounds, and methods for preparing these compounds. The compounds of this invention are useful for the treatment of inflammation, osteoarthritis, rheumatoid arthritis, psoriasis, Crohn's disease, inflammatory bowel disease, hyperproliferative diseases (such as cancer), autoimmune diseases, and for the treatment of other cytokine-mediated diseases.

2. Description of the State of the Art

A number of chronic and acute inflammatory conditions have been associated with the overproduction of pro-inflammatory cytokines. Such cytokines include but are not limited to tumor necrosis factor alpha (TNF-α), interleukin 1 beta (IL-1β), interleukin 8 (IL-8) and interleukin 6 (IL-6). Rheumatoid Arthritis (RA) is a chronic disease where TNF-α and IL-1β are implicated in the onset of the diseases and in the progression of the bone and joint destruction seen with this debilitating condition. Recently approved therapeutic treatments for RA have included a soluble TNF-α receptor (ENBREL®) and an IL-1 receptor antagonist (anakinra). These treatments work by blocking the ability of their respective cytokines to bind to their natural receptors. Alternative methods to treat cytokine-mediated diseases are currently under investigation. One such method involves inhibition of the signaling pathway that regulates the synthesis and production of pro-inflammatory cytokines such as p38.

P38 (also CSBP or RK) is a serine/threonine mitogen-activated protein kinase (MAPK) that has been shown to regulate pro-inflammatory cytokines. P38 was first identified as a kinase which becomes tyrosine phosphorylated in mouse monocytes following treatment with lipopolysaccharide (LPS). A link between p38 and the response of cells to cytokines was first established by Saklatvala J., et al. (Cell, 78:1039-1049 (1994)), who showed that IL-1 activates a protein kinase cascade that results in the phosphorylation of the small heat shock protein, Hsp27, probably by mitogen-activated protein activated protein kinase 2 (MAPKAP kinase-2). Analysis of peptide sequences derived from the purified kinase indicated that it was related to the p38 MAPK activated by LPS in mouse monocytes (Han, J., et al., Science, 265:808-811 (1994)). At the same time it was shown that p38 MAPK was itself activated by an upstream kinase in response to a variety of cellular stresses, including exposure to UV radiation and osmotic shock, and the identity of the kinase that directly phosphorylates Hsp27 was confirmed as MAPKAP kinase-2 (Rouse, J., et al., Cell, 78:1027-1037 (1994)). Subsequently, workers at SmithKline Beecham showed that p38 MAPK was the molecular target of a series of pyridinylimidazole compounds that inhibited the production of TNF from LPS-challenged human monocytes (Lee, J., et al., Nature, 372:739-746). This was a key discovery which led to the development of a number of selective inhibitors of p38 MAPK and the elucidation of its role in cytokine signaling.

It is now known that multiple forms of p38 MAPK (α, β, γ, δ), each encoded by a separate gene, form part of a kinase cascade involved in the response of cells to a variety of stimuli, including osmotic stress, UV light and cytokine mediated events. These four isoforms of p38 are thought to regulate different aspects of intracellular signaling. Its activation is part of a cascade of signaling events that lead to the synthesis and production of pro-inflammatory cytokines such as TNF-α. P38 functions by phosphorylating downstream substrates that include other kinases and transcription factors. Agents that inhibit p38 kinase have been shown to block the production of cytokines including, but not limited, to TNF-α, IL-6, IL-8 and IL-1β in vitro and in vivo models (Adams, J. L., et al., Progress in Medicinal Chemistry, 38:1-60 (2001)).

Peripheral blood monocytes (PBMCs) have been shown to express and secrete pro-inflammatory cytokines when stimulated with lipopolysaccharide (LPS) in vitro. P38 inhibitors efficiently block this effect when PBMCs are pretreated with such compounds prior to stimulation with LPS (Lee, J. C., et al., Int. J. Immunopharmacol., 10:835-843 (1988)). The efficacy of p38 inhibitors in animal models of inflammatory disease has prompted an investigation of the underlying mechanism(s) which could account for the effect of these inhibitors. The role of p38 in the response of cells to IL-1 and TNF has been investigated in a number of cells systems relevant to the inflammatory response using a pyridinyl imidazole inhibitor, such as: endothelial cells and IL-8 (Hashimoto, S., et al., J. Pharmacol. Exp. Ther., 293: 370-375 (2001)); fibroblasts and IL-6/GM-CSF/PGE2 (Beyaert, R., et al., EMBO J., 15:1914-1923 (1996)); neutrophils and IL-8 (Albanyan, E. A., et al., Infect. Immun., 68: 2053-2060 (2000)); macrophages and IL-1 (Caivano, M. and Cohen, P., J. Immunol., 164: 3018-3025 (2000)); and smooth muscle cells and RANTES (Maruoka, S., et al., Am. J. Respir. Crit. Care Med., 161:659-668 (1999)). The destructive effects of many disease states are caused by the over production of pro-inflammatory cytokines. The ability of p38 inhibitors to regulate this overproduction makes them excellent candidates for disease modifying agents.

Inhibitors of p38 are active in a variety of widely recognized disease models. Inhibitors of p38 show positive effects in a number of standard animal models of inflammation including rat collagen-induced arthritis (Jackson, J. R., et al., J. Pharmacol. Exp. Ther., 284:687-692 (1998)); rat adjuvant-induced arthritis (Badger, A. M., et al., Arthritis Rheum., 43:175-183 (2000); Badger, A. M., et al., J. Pharmacol. Exp. Ther., 279: 1453-1461 (1996)); and carrageenan-induced paw edema in the mouse (Nishikori, T., et al., Eur. J. Pharm., 451:327-333 (2002)). Molecules that block the function of p38 have been shown to be effective in inhibiting bone resorption, inflammation, and other immune and inflammation-based pathologies in these animal models. Thus, a safe and effective p38 inhibitor would provide a means to treat debilitating diseases that can be regulated by modulation of p38 signaling including, but not limited to, RA.

P38 inhibitors are well known to those skilled in the art. Reviews of early inhibitors have helped establish the structure-activity relationships important for enhanced activity both in vitro and in vivo (Salituro, E. G., et al., Current Medicinal Chemistry, 6: 807-823 (1999) and Foster, M. L., et al., Drug News Perspect., 13:488-497 (2000)). More contemporary reviews have focused on the structural diversity of new inhibitors being explored as p38 inhibitors (Boehm, J. D. and Adams, J. L., Exp. Opin. Ther. Patents, 10:25-37 (2000)).

### SUMMARY OF THE INVENTION

This invention provides compounds and pharmaceutical compositions containing said compounds which inhibit p38 alpha and associated p38 mediated events such as cytokine production. Such compounds have utility as therapeutic agents for diseases that can be treated by the inhibition of the p38 signaling pathway. In general, the invention relates to p38 inhibitors of the general Formula I:

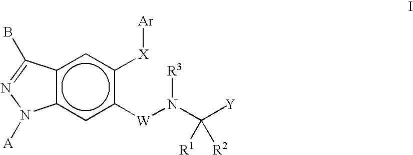

and metabolites, solvates, tautomers, resolved enantiomers, diastereomers, and pharmaceutically acceptable salts and prodrugs thereof, wherein A, B, W, X Ar, Y, R¹, R² and R³ are as defined herein.

In certain embodiments, this invention relates to compounds of the general Formula Ia

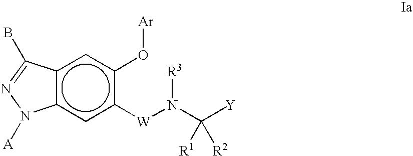

and metabolites, solvates, tautomers, resolved enantiomers, diastereomers, salts and pharmaceutically acceptable prodrugs thereof, wherein A, B, Y, W, Ar, R¹, R² and R³ are as defined herein.

In other embodiments, this invention relates to compounds of the general Formula Ib

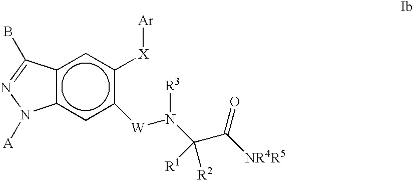

and metabolites, solvates, tautomers, resolved enantiomers, diastereomers, salts and pharmaceutically acceptable prodrugs thereof, wherein A, B, X, W, Ar, R¹, R², R³, R⁴ and R⁵ are as defined herein.

In other embodiments, this invention relates to compounds of the general Formula Ic

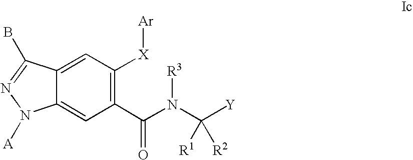

and metabolites, solvates, tautomers, resolved enantiomers, diastereomers, salts and pharmaceutically acceptable prodrugs thereof, wherein A, B, Ar, X, Y, R¹, R² and R³ are as defined herein.

In other embodiments, this invention relates to compounds of the general Formula Id and metabolites, solvates, tautomers, resolved enantiomers, diastereomers, salts and pharmaceutically acceptable prodrugs thereof,

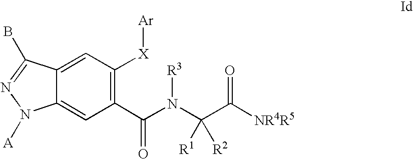

wherein A, B, X, Ar, R¹, R², R³, R⁴ and R⁵ are as defined herein.

In other embodiments, this invention relates to compounds of the general Formula Ie

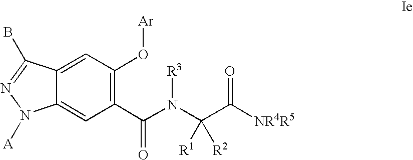

and metabolites, solvates, tautomers, resolved enantiomers, diastereomers, salts and pharmaceutically acceptable prodrugs thereof, wherein A, B, Ar, R¹, R², R³, R⁴ and R⁵ are as defined herein.

In a further aspect the present invention provides compositions that inhibit the production of cytokines such as TNF-α, IL-1, IL-6 and IL-8 comprising one or more compounds of Formulas I.

In a further aspect the present invention provides a method of treating diseases or medical conditions mediated by cytokines which comprises administering to a human or warm-blooded animal an effective amount of a compound of claim 1, or a pharmaceutically acceptable salt or prodrug thereof or a pharmaceutical composition comprising said compound, in an amount effective to treat said cytokine-mediated disease.

In a further aspect the present invention provides a method of inhibiting the production of cytokines such as TNF-α, IL-1, IL-6 and IL-8, which comprises administering to a human or warm-blooded animal a compound of claim 1, or a pharmaceutically acceptable salt or prodrug thereof or a pharmaceutical composition comprising said compound, in an amount effective to inhibit the production of such cytokines.

In a further aspect the present invention provides a method of providing a p38 kinase inhibitory effect comprising administering to a human or warm-blooded animal a compound of claim 1, or a pharmaceutically acceptable salt or prodrug thereof or a pharmaceutical composition comprising said compound, in an amount effective to provide a p38 kinase inhibitory effect.

In a further aspect the present invention provides treating or preventing a p38-mediated condition, comprising administering to a human or animal in need thereof a compound of claim 1, or a pharmaceutically acceptable salt or prodrug thereof or a pharmaceutical composition comprising said compound, in an amount effective to treat or prevent said p38-mediated condition. P38-mediated conditions that can be treated according to the methods of this invention include, but are not limited to, inflammatory disease, autoimmune disease, destructive bone disorder, hyperproliferative disorder, infectious disease, viral disease, and neurodegenerative disease.

The compounds of this invention are also useful in methods for preventing cell death and hyperplasia and therefore may be used to treat or prevent reperfusion/ischemia in stroke, heart attacks, and organ hypoxia. The compounds of this invention are also useful in methods for preventing thrombin-induced platelet aggregation.

The invention also relates to pharmaceutical compositions comprising one or more compounds of Formula I or a pharmaceutically acceptable prodrug, pharmaceutically active metabolite, or pharmaceutically acceptable salt thereof. The inventive compounds may be used advantageously in combination with other known therapeutic agents.

Another aspect of the invention includes articles of manufacture, i.e., kits, comprising a compound of Formula I, a container, and a package insert or label indicating a treatment.

Also provided are methods of preparing compounds of Formula I.

Additional advantages and novel features of this invention shall be set forth in part in the description that follows, and in part will become apparent to those skilled in the art upon examination of the following specification or may be learned by the practice of the invention. The advantages of the invention may be realized and attained by means of the instrumentalities, combinations, compositions, and methods particularly pointed out in the appended claims.

### BRIEF DESCRIPTION OF THE FIGURES

The accompanying drawings, which are incorporated herein and form a part of the specification, illustrate non-limiting embodiments of the present invention, and together with the description, serve to explain the principles of the invention.

In the Figures:

FIG. 1 shows a reaction scheme for the synthesis of compounds of Formula Ia wherein W is C(═O) and Y is CO₂Me.

FIG. 2 shows reaction schemes for the syntheses of Formula Ia wherein W is C(═O) and Y is CO₂NH₂.

FIG. 3 shows a reaction scheme for the synthesis of compounds of Formula I wherein X is O, S, NH, NCH₃, SO or SO₂.

FIG. 4 shows a reaction scheme for the synthesis of compounds of Formula Id wherein X is C═NOR⁴.

FIG. 5 shows a reaction scheme for the synthesis of compounds of Formula Id wherein X is CH(OH) or C(═O).

FIG. 6 shows a reaction scheme for the synthesis of compounds of Formula Id wherein X is CH₂.

### DETAILED DESCRIPTION OF THE INVENTION

The compounds of the general Formulas I are useful for inhibiting p38 alpha and associated p38 mediated events such as cytokine production. Such compounds have utility as therapeutic agents for diseases that can be treated by the inhibition of the p38 signaling pathway. In general, the invention relates to compounds of the general Formula I:

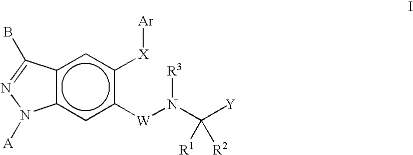

and metabolites, solvates, tautomers, resolved enantiomers, diastereomers, salts and pharmaceutically acceptable prodrugs thereof, wherein:

A is H, an amine protecting group, C₁-C₈ alkyl, C₂-C₈ alkenyl, C₂-C₈ alkynyl, saturated or partially unsaturated cycloalkyl, saturated or partially unsaturated heterocycloalkyl, aryl or heteroaryl, wherein said alkyl, alkenyl, alkynyl, cycloalkyl, heterocycloalkyl, aryl and heteroaryl are optionally substituted with one or more groups independently selected from F, Cl, Br, I, CN, ═O (with the proviso that it is not substituted on an aryl or heteroaryl), ═NOR⁴ (with the proviso that it is not substituted on an aryl or heteroaryl), —NR⁴R⁵, —NR⁴(C═O)R⁵, —NR⁴C(═O)NR⁵R⁷, —CR⁵═NOR⁴—SO₂R⁶, —SOR⁶, —SR⁵, —SO₂NR⁴R⁵, —OR⁴, —(C═O)R⁴, —(C═O)OR⁴, —O—(C═O)R⁴, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I;

B is H, —NH₂, —NHMe, —NMe₂, —CH₃, —CF₃, —CH₂OH, cyclopropyl, C₁-C₃ alkyl, —OH, CN, F, Cl, Br or I, wherein said alkyl is optionally substituted with one or more groups independently selected from F, Cl, Br and I;

W is C(═O) or SO₂;

X is O, S, SO, SO₂, NH, NCH₃, C(═O), CH₂, CH(CH₃), C(CH₃)₂, C═NOR⁴, C═CR⁴ or CHOR⁴;

Y is —C(═O)R⁴, —C(═O)OR⁴, —C(═O)NR⁴R⁵, —CR⁴R⁵OR⁷, —C(═O)NR⁴OR⁵ or —C(═O)NR⁴NR⁵R⁷;

R¹ is C₁-C₈ alkyl, C₂-C₈ alkenyl, C₂-C₈ alkynyl, C₁-C₈ heteroalkyl, saturated or partially unsaturated cycloalkyl, or saturated or partially unsaturated heterocycloalkyl, wherein said alkyl, alkenyl, alkynyl, heteroalkyl, cycloalkyl and heterocycloalkyl are optionally substituted with one or more groups independently selected from F, Cl, Br, I, CN, ═O, ═NOR⁴, —NR⁴R⁵, —NR⁴(C═O)R⁵, —NR⁴C(═O)NR⁵R⁷, —CR⁵═NOR⁴—SO₂R⁶, —SOR⁶, —SR⁵, —SO₂NR⁴R⁵, —OR⁴, —(C═O)R⁴, —(C═O)OR⁴, —O—(C═O)R⁴, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I;

R² and R⁶ are independently alkyl, alkenyl, alkynyl, saturated or partially unsaturated cycloalkyl, saturated or partially unsaturated heterocycloalkyl, aryl or heteroaryl, wherein said alkyl, alkenyl, alkynyl, cycloalkyl, heterocycloalkyl, aryl and heteroaryl are optionally substituted with one or more groups independently selected from F, Cl, Br, I, CN, ═O (with the proviso that it is not substituted on an aryl or heteroaryl), ═NOR⁴ (with the proviso that it is not substituted on an aryl or heteroaryl), —NR⁴R⁵, —NR⁴(C═O)R⁵, —NR⁴C(═O)NR⁵R⁷, —CR⁵═NOR⁴—SO₂R⁶, —SOR⁶, —SR⁵, —OR⁴, —(C═O)R⁴, —(C═O)OR⁴, —O—(C═O)R⁴, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br, I and OH,

or R¹ and R² together with the atom to which they are attached form a saturated or partially unsaturated 3-10 membered carbocyclic ring or a saturated or partially unsaturated 3-10 membered heterocyclic ring having 1 or more heteroatoms, wherein said carbocyclic and heterocyclic rings are optionally substituted with one or more groups independently selected from F, Cl, Br, I, CN, ═O, ═NOR⁴, —NR⁴R⁵, —NR⁴(C═O)R⁵, —NR⁴C(═O)NR⁵R⁷, —CR⁵═NOR⁴—SO₂R⁶, —SOR⁶, —SR⁵, SO₂NR⁴R⁵, —OR⁴, —(C═O)R⁴, —(C═O)OR⁴, —O—(C═O)R⁴, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I;

R³ is H, C₁-C₄ alkyl, C₃-C₆ cycloalkyl or —OR⁴, wherein said alkyl and cycloalkyl are optionally substituted with one or more groups independently selected from F, Cl, Br, I, and alkyl;

R⁴, R⁵ and R⁷ are independently H, alkyl, alkenyl, alkynyl, saturated or partially unsaturated cycloalkyl, saturated or partially unsaturated heterocycloalkyl, aryl, or heteroaryl, wherein said alkyl, alkenyl, alkynyl, cycloalkyl, heterocycloalkyl, aryl and heteroaryl are optionally substituted with one or more groups independently selected from F, Cl, Br, I, CN, ═O (with the proviso that it is not substituted on an aryl or heteroaryl), ═NOR⁸ (with the proviso that it is not substituted on an aryl or heteroaryl), —NR⁸R⁹, —NR⁸(C═O)R⁹, —NR⁸C(═O)NR⁹R¹⁰, —CR⁹═NOR⁸—SO₂R⁶, —SOR⁶, —SR⁹, —SO₂NR⁸R⁹, —OR⁸, —(C═O)R⁸, —(C═O)OR⁸, —O—(C═O)R⁸, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I,

or R⁴ and R⁵ together with the atom to which they are attached form a saturated or partially unsaturated 4-8 membered carbocyclic ring or a saturated or partially unsaturated 4-8 membered heterocyclic ring having 1 or more heteroatoms, wherein said carbocyclic and heterocyclic rings are optionally substituted with one or more groups independently selected from F, Cl, Br, I, CN, ═O, ═NOR⁸, —NR⁸R⁹, —NR⁸(C═O)R⁹, —NR⁸C(═O)NR⁹R¹⁰, —CR⁹═NOR⁸—SO₂R¹¹, —SOR¹¹, —SR⁹, SO₂NR⁸R⁹, —OR⁸, —(C═O)R⁸, —(C═O)OR⁸, —O—(C═O)R⁸, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I, and wherein said carbocyclic and heterocyclic rings are optionally fused to an aromatic ring,

or R⁵ and R⁷ together with the atom to which they are attached form a saturated or partially unsaturated 4-8 membered heterocyclic ring having 1 or more heteroatoms, wherein said heterocyclic ring is optionally substituted with one or more groups independently selected from F, Cl, Br, I, CN, ═O, ═NOR⁸, —NR⁸R⁹, —NR⁸(C═O)R⁹, —NR⁸C(═O)NR⁹R¹⁰, —CR⁹═NOR⁸—SO₂R¹¹, —SOR¹¹, —SR⁹, SO₂NR⁸R⁹, —OR⁸, —(C═O)R⁸, —(C═O)OR⁸, —O—(C═O)R⁸, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I, and wherein said heterocyclic ring is optionally fused to an aromatic ring;

R⁸, R⁹ and R¹⁰ are independently H, alkyl, alkenyl, alkynyl, saturated or partially unsaturated cycloalkyl, saturated or partially unsaturated heterocycloalkyl, aryl, or heteroaryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I;

R¹¹ is alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br, and I; and

Ar is aryl or heteroaryl, wherein said aryl and heteroaryl are optionally substituted with one or more groups independently selected from —NH₂, —NHMe, —NMe₂, —CH₃, —CF₃, —CH₂OH, cyclopropyl, —OH, CN, F, Cl, Br, I, and C₁-C₃ alkyl, wherein said alkyl is optionally substituted with one or more groups independently selected from F, Cl, Br and I.

It was discovered that, for compounds of Formula I having a terminal amide group (i.e., where Y is —C(═O)NR⁴R⁵), the presence of a quaternary carbon in the position directly adjacent to Y provides improved pharmacokinetic properties by, for example, reducing the rate of plasma metabolism of such compounds to less potent or inactive species, thereby increasing the half-life of the active Formula I compound. These compounds have been found to have increased stability against certain enzymes in vivo, including esterases and amidases, and minimized hydrolysis of the terminal amide group. As a result, compounds of Formula I having a terminal amide may allow a more attractive therapeutic schedule—for example, by allowing treatment at lower doses or with fewer daily doses.

The term “quaternary carbon” as used herein refers to a carbon atom bonded to four other atoms, other than hydrogen, through single bonds.

In certain embodiments, provided are compounds of Formula I wherein X is O.

In certain embodiments, provided are compounds of Formula I wherein W is C═O.

In certain embodiments, provided are compounds of Formula I wherein B is H.

In certain embodiments, provided are compounds of Formula I wherein R³ is H.

In certain embodiments, provided are compounds of Formula I wherein A is H or C₁-C₈ alkyl, C₂-C₈ alkenyl or C₂-C₈ alkynyl, wherein said alkyl, alkenyl and alkynyl are optionally substituted with one or more groups independently selected from F, Cl, Br, I, CN, —NR⁴R⁵, —NR⁴(C═O)R⁵, —OR⁴, —(C═O)R⁴, —(C═O)OR⁴, —O—(C═O)R⁴, and alkyl. In certain embodiment, A is H,C₁-C₈ alkyl, C₂-C₈ alkenyl or C₂-C₈ alkynyl, wherein said alkyl, alkenyl and alkynyl are optionally substituted with one or more groups independently selected from F, Cl, Br, I, alkyl and OR⁴. For example, in certain embodiments, A is H or alkyl optionally substituted with one or more groups independently selected from alkyl, OH and F. Exemplary embodiments include, but are not limited to, H, methyl, ethyl, n-propyl, isopropyl, butyl, CH₂F, CH₂CH₂F, CH₂C(CH₃)₂F, CH₂OH, CH₂CH₂OH, CH₂C(CH₃)₂OH, CH₂CF₃, CH₂CH═CH₂, and the like. Particular embodiments include methyl, isopropyl, CH₂C(CH₃)₂F, CH₂CH₂OH, and CH₂C(CH₃)₂OH.

In certain embodiments, provided are compounds of Formula I wherein Ar is aryl optionally substituted by one or more groups independently selected from —NH₂, —NHMe, —NMe₂, —CH₃, —CF₃, —CH₂OH, cyclopropyl, C₁-C₃ alkyl, —OH, CN, F, Cl, Br and I. In particular embodiment, Ar is phenyl optionally substituted by one or more groups independently selected from —NH₂, —NHMe, —NMe₂, —CH₃, —CF₃, —CH₂OH, cyclopropyl, C₁-C₃ alkyl, —OH, CN, F, Cl, Br and I, wherein said alkyl is optionally substituted with one or more groups independently selected from F, Cl, Br and I. Exemplary embodiments of substituted Ar include, but are not limited to, phenyl, 2-chlorophenyl, 3-chlorophenyl, 4-chlorophenyl, 2-fluorophenyl, 3-fluorophenyl, 4-fluorophenyl, 2,4-difluorophenyl, 2,5-difluorophenyl, 2-methylphenyl, 3-methylphenyl, 4-methylphenyl, 2-methoxyphenyl, 3-methoxyphenyl, 4-methoxyphenyl, 2-trifluoromethylphenyl, 3-trifluoromethylphenyl, 4-trifluoromethylphenyl, 2-trifluoromethoxyphenyl, 3-trifluoromethoxyphenyl, 4-trifluoromethoxyphenyl, and 4-(3,5-bis-trifluoromethylphenyl). In particular embodiments, Ar is 2,4-difluorophenyl.

In certain embodiments, provided are compounds of Formula I wherein Y is —C(═O)NR⁴R⁵, and R⁴ and R⁵ are independently H, alkyl, alkenyl, or alkynyl, wherein said alkyl, alkenyl, and alkynyl are optionally substituted with one or more groups independently selected from OR⁸, or R⁴ and R⁵ together with the atoms to which they are attached form a 5 to 6 membered heterocyclic ring, wherein said heterocyclic ring is optionally substituted with one or more groups independently selected from OR⁸.

For example in certain embodiments, provided are compounds of Formula I wherein Y is —C(═O)NR⁴R⁵ and R⁴ and R⁵ are independently H or an alkyl group optionally substituted with OH or O-alkyl. Exemplary embodiments include, but are not limited to, —C(═O)NH₂, —C(═O)NHCH₃, —C(═O)NHCH₂CH₃, —C(═O)N(CH₃)₂, —C(═O)N(CH₂CH₃)₂, —C(═O)NCH(CH₃)₂, —C(═O)NH(CH₂CH₂OH), and —C(═O)NHCH₂CH₂OCH₃. Particular embodiments of Y include —C(═O)NH₂, —C(═O)NHCH₃, —C(═O)N(CH₃)₂, and —C(═O)NH(CH₂CH₂OH).

For example in certain embodiments, provided are compounds of Formula I wherein Y is —C(═O)NR⁴R⁵ and R⁴ and R⁵ together with the atoms to which they are attached form a 5 to 6 membered heterocyclic ring. Exemplary embodiments of such Y groups include, but are not limited to,

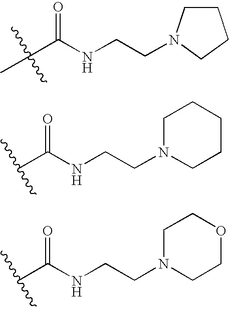

In other embodiments, Y is —C(═O)OR⁴. Particular embodiments include, but are not limited to, —C(═O)OH, —C(═O)OCH₃, —C(═O)OCH₂CH₃, and the like.

In certain embodiment, provided are compounds of Formula I wherein R¹ is C₁-C₈ alkyl, C₂-C₈ alkenyl, or C₂-C₈ alkynyl, wherein said alkyl, alkenyl, and alkynyl are optionally substituted with one or more groups independently selected from F, Cl, Br, I, CN, —NR⁴R⁵, —NR⁴(C═O)R⁵, —OR⁴, —(C═O)R⁴, —(C═O)OR⁴, —O—(C═O)R⁴, alkyl and cycloalkyl Exemplary embodiments of R¹ include, but are not limited to, C₁-C₈ alkyl optionally substituted with one or more groups independently selected from F, Cl, Br, I, alkyl and cycloalkyl.

Particular embodiments of R¹ include CH₃, CH₂CH₃, CH₂F, CHF₂, CF₃, CH₂-(cyclopropyl) and CH₂CH₂— (cyclopropyl).

In certain embodiment, provided are compounds of Formula I wherein R² is C₁-C₈ alkyl, C₂-C₈ alkenyl, or C₂-C₈ alkynyl, wherein said alkyl, alkenyl, and alkynyl are optionally substituted with one or more groups independently selected from F, Cl, Br, I, CN, —NR⁴R⁵, —NR⁴(C═O)R⁵, —OR⁴, —(C═O)R⁴, —(C═O)OR⁴, —O—(C═O)R⁴ and alkyl. Exemplary embodiments of such R² groups include, but are not limited to, C₁-C₈ alkyl optionally substituted with NR⁴R⁵, wherein R⁴ and R⁵ are independently H or a C₁-C₈ alkyl group optionally substituted with one or more groups independently selected from OR⁸.

Particular embodiments of R² include, but are not limited to, CH₂CH₂NH₂, CH₂CH₂NHCH₃, CH₂CH₂NH(CH₂CH₃), CH₂CH₂NH(CH₂CH₂CH₃), CH₂CH₂NHCH(CH₃)₂, CH₂CH₂N(CH₃)₂, CH₂CH₂N(CH₂CH₃)₂, CH₂CH₂N(CH₂CH₂CH₃)₂, CH₂CH₂N(CH₃)CH₂CH₂OH, and CH₂CH₂N(CH₃)CH₂CH₂OCH₃.

As a further example, provided are compounds of Formula I wherein R² is alkyl optionally substituted with NR⁴R⁵, wherein R⁴ and R⁵ together with the atoms to which they are attached form a 5 to 6 membered heterocyclic ring, and said heterocyclic ring is optionally substituted with one or more groups independently selected from OR⁸. For example, in certain embodiments R² is selected from the structures

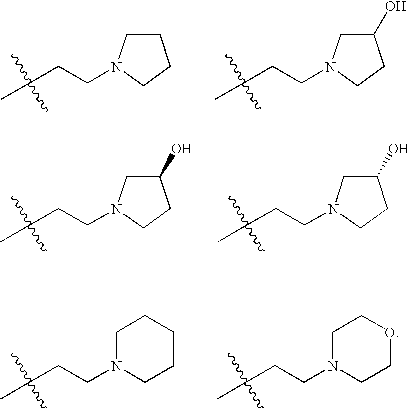

In a specific embodiment, this invention relates to compounds of the general Formula Ia:

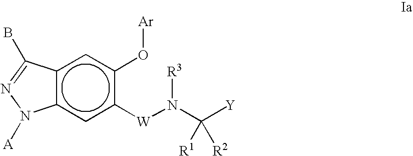

and metabolites, solvates, tautomers, resolved enantiomers, diastereomers, pharmaceutically acceptable salts and pharmaceutically acceptable prodrugs thereof, wherein A, B, Y, W, Ar, R¹, R² and R³ are as defined above. In one embodiment, Y is —C(═O)R⁴, —C(═O)OR⁴ or —C(═O)NR⁴R⁵. In a particular embodiment, Y is —C(═O)NH₂.

In one embodiment, R¹ is C₁-C₈ alkyl optionally substituted with one or more groups independently selected from F, Cl, Br, I, alkyl and cycloalkyl. In certain other embodiments, R¹ is methyl, fluoromethyl, difluoromethyl, trifluoromethyl, ethyl or cyclopropylmethyl.

In another embodiment, R² is C₁-C₈ alkyl optionally substituted with —NR⁴R⁵ or a heterocycle. In yet another embodiment, R² is C₁-C₆ alkyl substituted with —NMe₂, —NEt₂, —N(CH₃)(CH₂CH₃) or pyrrolidinyl.

In other embodiments, A is C₁-C₈ alkyl optionally substituted with one or more alkyl groups. In a particular embodiment, A is —CH₂CH(CH₃)₂.

In other embodiments, this invention relates to compounds of the general Formula Ib

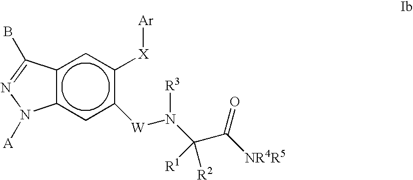

and metabolites, solvates, tautomers, resolved enantiomers, diastereomers, pharmaceutically acceptable salts and pharmaceutically acceptable prodrugs thereof, wherein A, B, X, W, Ar, R¹, R², R³, R⁴ and R⁵ are as defined above. In one embodiment, NR⁴R⁵ is NH₂. In one embodiment, R¹ is C₁-C₈ alkyl optionally substituted with one or more groups independently selected from F, Cl, Br, I, alkyl and cycloalkyl. In certain other embodiments, R¹ is methyl, fluoromethyl, difluoromethyl, trifluoromethyl, ethyl or cyclopropylmethyl.

In another embodiment, R² is C₁-C₈ alkyl optionally substituted with —NR⁴R⁵ or a heterocycle. In yet another embodiment, R² is C₁-C₆ alkyl substituted with —NMe₂, —NEt₂, —N(CH₃)(CH₂CH₃) or pyrrolidinyl.

In other embodiments, A is C₁-C₈ alkyl optionally substituted with one or more alkyl groups. In a particular embodiment, A is —CH₂CH(CH₃)₂.

In other embodiments, this invention relates to compounds of the general Formula Ic

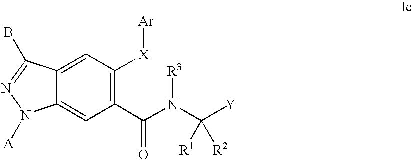

and metabolites, solvates, tautomers, resolved enantiomers, diastereomers, pharmaceutically acceptable salts and pharmaceutically acceptable prodrugs thereof, wherein A, B, Ar, X, Y, R¹, R² and R³ are as defined above. In one embodiment, Y is —C(═O)R⁴, —C(═O)OR⁴ or —C(═O)NR⁴R⁵. In a particular embodiment, Y is —C(═O)NH₂.

In one embodiment, R¹ is C₁-C₈ alkyl optionally substituted with one or more groups independently selected from F, Cl, Br, I, alkyl and cycloalkyl. In certain other embodiments, R¹ is methyl, fluoromethyl, difluoromethyl, trifluoromethyl, ethyl or cyclopropylmethyl.

In another embodiment, R² is C₁-C₈ alkyl optionally substituted with —NR⁴R⁵ or a heterocycle. In yet another embodiment, R² is C₁-C₆ alkyl substituted with —NMe₂, —NEt₂, —N(CH₃)(CH₂CH₃) or pyrrolidinyl.

In other embodiments, A is C₁-C₈ alkyl optionally substituted with one or more alkyl groups. In a particular embodiment, A is —CH₂CH(CH₃)₂

In other embodiments, this invention relates to compounds of the general Formula Id

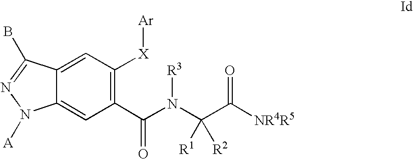

and metabolites, solvates, tautomers, resolved enantiomers, diastereomers, pharmaceutically acceptable salts and pharmaceutically acceptable prodrugs thereof, wherein A, B, X, Ar, R¹, R², R³, R⁴ and R⁵ are as defined above. In one embodiment, R¹ is C₁-C₈ alkyl optionally substituted with one or more groups independently selected from F, Cl, Br, I, alkyl and cycloalkyl. In certain other embodiments, R¹ is methyl, fluoromethyl, difluoromethyl, trifluoromethyl, ethyl or cyclopropylmethyl.

In another embodiment, R² is C₁-C₈ alkyl optionally substituted with —NR⁴R⁵ or a heterocycle. In yet another embodiment, R² is C₁-C₆ alkyl substituted with —NMe₂, —NEt₂, —N(CH₃)(CH₂CH₃) or pyrrolidinyl.

In other embodiments, A is C₁-C₈ alkyl optionally substituted with one or more alkyl groups. In a particular embodiment, A is —CH₂CH(CH₃)₂.

In other embodiments, this invention relates to compounds of the general Formula Ie and metabolites, solvates, tautomers, resolved enantiomers, diastereomers, pharmaceutically acceptable salts and pharmaceutically acceptable prodrugs thereof

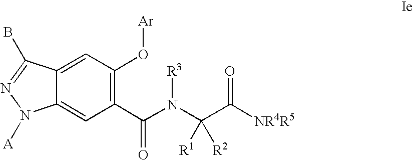

wherein A, B, Ar, R¹, R², R³, R⁴ and R⁵ are as defined above. In one embodiment, R¹ is C₁-C₈ alkyl optionally substituted with one or more groups independently selected from F, Cl, Br, I, alkyl and cycloalkyl. In certain other embodiments, R¹ is methyl, fluoromethyl, difluoromethyl, trifluoromethyl, ethyl or cyclopropylmethyl.

In another embodiment, R² is C₁-C₈ alkyl optionally substituted with —NR⁴R⁵ or a heterocycle. In yet another embodiment, R² is C₁-C₆ alkyl substituted with —NMe₂, —NEt₂, —N(CH₃)(CH₂CH₃) or pyrrolidinyl.

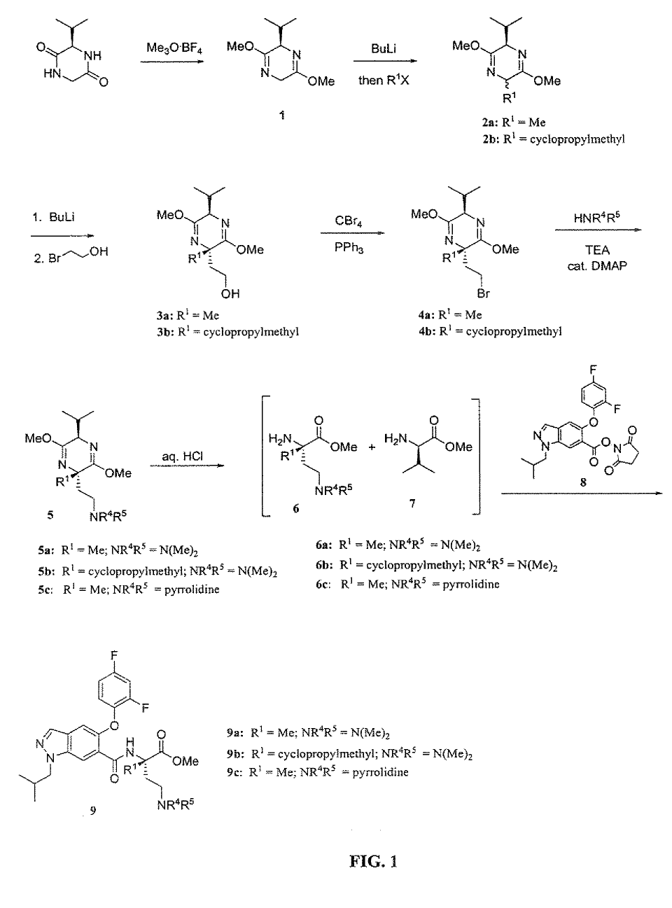

FIG. 1 shows one embodiment for the preparation of compounds of Formula I (specifically, compounds 9a, 9b and 9c), wherein a chiral quaternary amino acid 6 is coupled with an indazole derivative 8 to provide a compound 9 having a quaternary carbon in the position alpha to the Y group. A more detailed description of the specific compounds 9a, 9b and 9c is provided in Examples 1-3. Indazole derivative (8) is prepared as described in Example 4. The chiral quaternary amino acid 6 was prepared from (R)-3-isopropylpiperazine-2,5-dione according to published methods (T. Ooi, et al., J. Am. Chem. Soc., 2000, 122, 5228-5229; C. Cativiela et al., Tetrahedron: Assymetry, 1998, 9, 3517-3599) and as described in detail in Examples 1-3. As used herein, the term “quaternary amino acid” refers to an amino acid having a quaternary carbon, wherein the amino functionality and the carboxylic acid functionality are bonded to the same quaternary carbon.

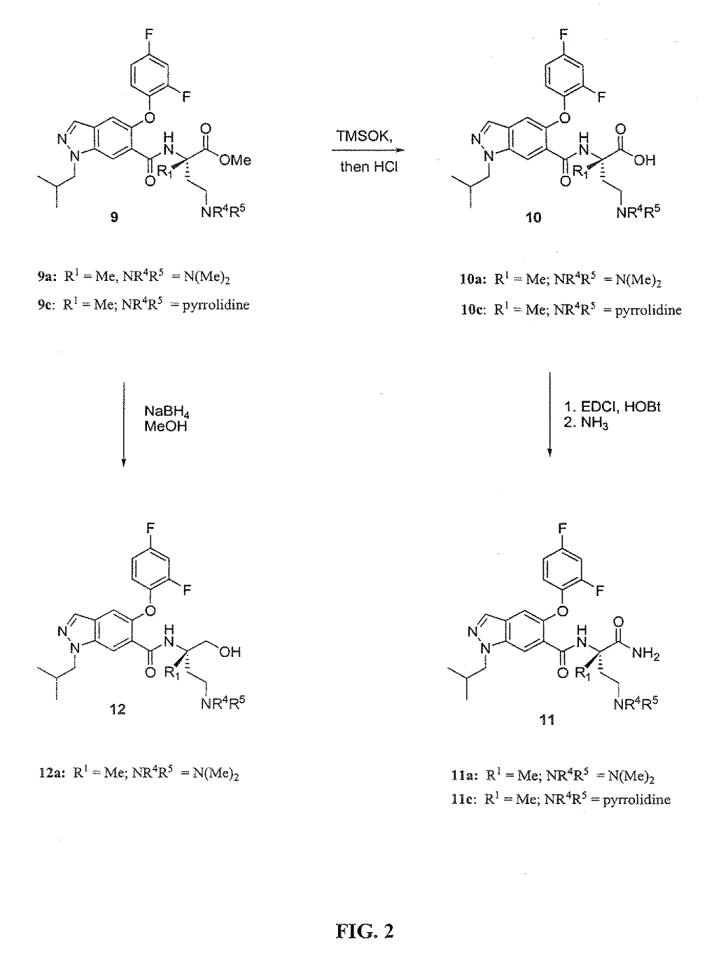

FIG. 2 shows methods of preparing compounds of Formula Ic, i.e., compounds 10a, 10c, 11a, 11c and 12a wherein Y is —COOH, —CH₂OH or —C(═O)NR⁴′R⁵′, and R⁴′ and R⁵′ are independently H, alkyl, alkenyl, alkynyl, saturated or partially unsaturated cycloalkyl, saturated or partially unsaturated heterocycloalkyl, aryl, or heteroaryl, wherein said alkyl, alkenyl, alkynyl, cycloalkyl, heterocycloalkyl, aryl and heteroaryl are optionally substituted with one or more groups independently selected from F, Cl, Br, I, CN, ═O (with the proviso that it is not substituted on an aryl or heteroaryl), ═NOR⁸ (with the proviso that it is not substituted on an aryl or heteroaryl), —NR⁸R⁹, —NR⁸(C═O)R⁹, —NR⁸C(═O)NR⁹R¹⁰, —CR⁹═NOR⁸—SO₂R⁶, —SOR⁶, —SR⁹, —SO₂NR⁸R⁹, —OR⁸, —(C═O)R, —(C═O)OR, —O—(C═O)R⁸, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I, or

R^{4′} and R⁵′ together with the atom to which they are attached form a saturated or partially unsaturated 4-8 membered carbocyclic ring or a saturated or partially unsaturated 4-8 membered heterocyclic ring having 1 or more heteroatoms, wherein said carbocyclic and heterocyclic rings are optionally substituted with one or more groups independently selected from F. Cl, Br, I, CN, ═O, ═NOR⁸, —NR⁸R⁹, —NR⁸(C═O)R⁹, —NR⁸C(═O)NR⁹R¹⁰, —CR⁹═NOR⁸—SO₂R⁶, —SOR⁶, —SR⁹, —SO₂NR⁸R⁹, —OR⁸, —(C═O)R⁸, —(C═O)OR⁸, —O—(C═O)R⁸, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I, and wherein said carbocyclic and heterocyclic rings are optionally fused to an aromatic ring The syntheses of compounds 10a, 10c, 11a, 11c and 12a are described in detail in Examples 5-7.

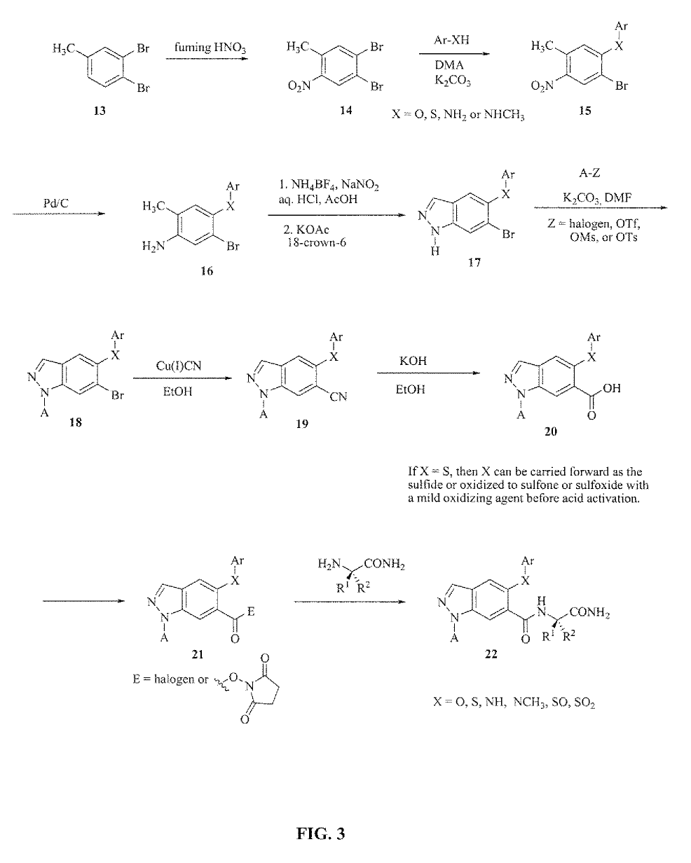

FIG. 3 shows one embodiment of a method for preparing compounds of Formula Id where X═O, S, NH, NCH₃, SO or SO₂. In one general synthetic process, compounds of Formula Ib are prepared as follows. Commercially available 1,2-dibromo-4-methylbenzene is treated with fuming nitric acid to afford the tetra-substituted intermediate (14). Treatment with a heteroatom-substituted aryl group (e.g., phenol, aniline, thiophenol), an inorganic base and heat leads to displacement of the more activated bromide to provide the nitro-substituted intermediate (15). Intermediate (15) can then be reduced, for example using palladium on carbon, to yield the para-substituted aniline (16). A standard diazotization and subsequent ring closure with base affords the N1-unsubstituted indazole (17). The N1-indazole intermediate can be readily alkylated with an electrophile (e.g., R—Br, R—I) to yield (18). Treatment of (18) with copper cyanide followed by basic aqueous work-up affords in two steps the key intermediate acid (20). If X=sulfur, then oxidation to the sulfoxide or sulfone can be achieved using standard methods, for example MCPBA, prior to the final coupling step. Classical amide bond formation by coupling with a quaternary amine as previously described leads to the desired product (22). The term “quaternary amine” as used herein refers to a compound having an amino functionality, wherein the amino functionality is bonded to a quaternary carbon (i.e., a carbon atom bonded to four other atoms other than hydrogen through single bonds).

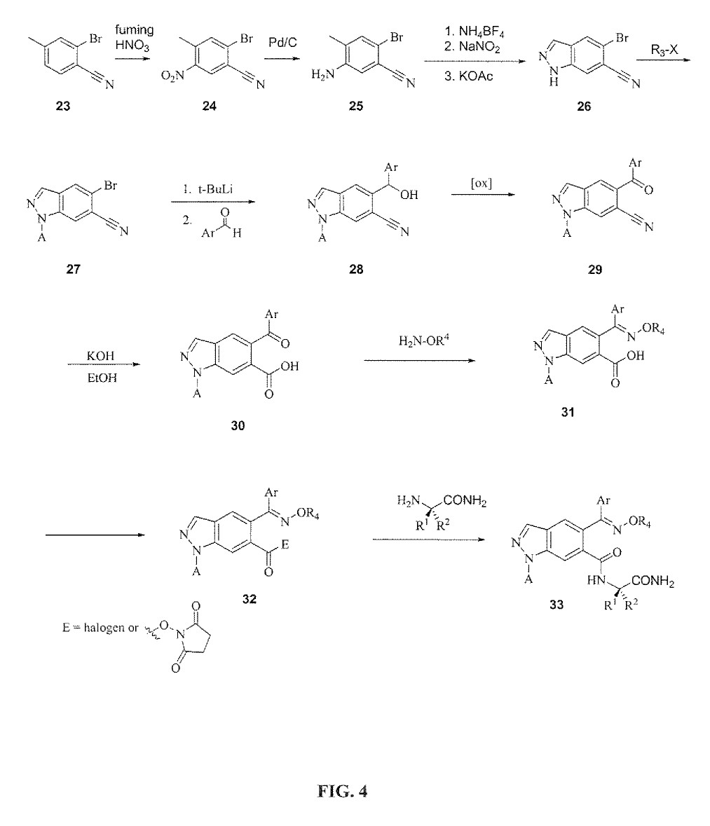

FIG. 4 shows one embodiment of a method for the synthesis of compounds of Formula Id wherein X is C═NOR⁴. Commercially available 2-bromo-4-methylbenzonitrile is treated with fuming nitric acid to afford the tetra-substituted intermediate (24). Intermediate (24) is then treated with palladium on carbon to reduce the nitro group (25) to NH₂. This is followed by previously described diazotization conditions and subsequent ring closure with base to afford the N1-unsubstituted indazole (26). The N1-indazole can be readily alkylated with an electrophile to yield (27) as previously described. Metal-halogen exchange followed by quenching with an electrophilic aldehyde yields intermediate (28). The alcohol can be oxidized to the ketone using, for example standard Swern conditions to produce (29). Hydrolysis of the nitrile in aqueous base affords intermediate (30). Treatment of this intermediate with an oxygen-alkylated or oxygen protected hydroxylamine gives (31). The acid functionality of intermediate (31) can then be converted to the activated intermediate (32) which is then reacted with a quaternary amine to afford final product (33).

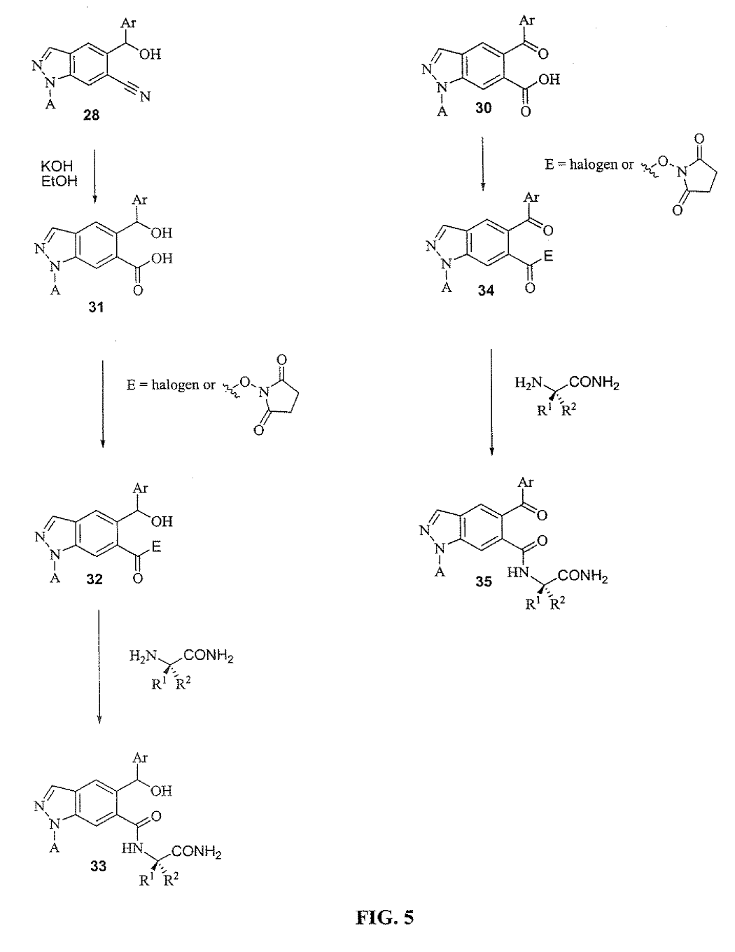

FIG. 5 shows an example of the synthesis of compounds having the general Formula Id where X═CHOH or C═O. In one general synthetic process, compounds of Formula Id wherein X═CHOH are prepared as follows. Starting with intermediate (28), the nitrile group can be hydrolyzed in aqueous base to form penultimate acid intermediate (31), which can be converted to the activated ester (32). Reaction of the activated ester (31) with a quaternary amine provides the product (33). In another general synthetic process shown in FIG. 5, compounds of Formula Id wherein X is C═O can be prepared from the ketone intermediate (30). Activation of ketone (30) to provide the activated ester (34), followed by treatment of activated ester (34) with a quaternary amine, affords the desired product (35).

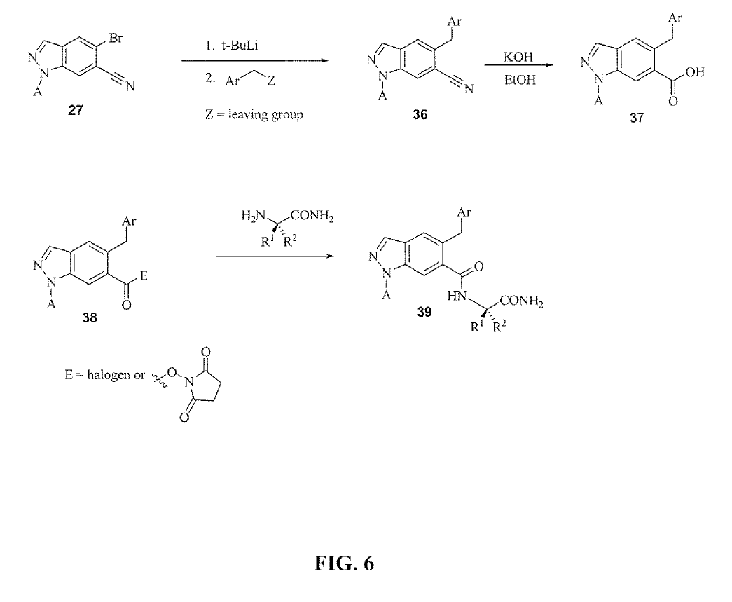

FIG. 6 shows an example of the synthesis of compounds having the general Formula Id where X═CH₂. In one general synthetic process, compounds of Formula Id are prepared as follows. Intermediate (27), prepared as described in FIG. 4, is treated with t-butyl lithium, for example in a metal-halogen exchange reaction, followed by quenching with an electrophilic benzyl group, such as benzyl bromide, to provide (36). After carbon bond formation, the nitrile is hydrolyzed to the acid, which can then be activated and coupled with a quaternary amine as described previously to afford the desired product (39).

The term “alkyl” as used herein refers to a saturated linear or branched-chain monovalent hydrocarbon radical of one to twelve carbon atoms, wherein the alkyl radical may be optionally substituted independently with one or more substituents described below. Examples of alkyl groups include, but are not limited to, methyl, ethyl, n-propyl, isopropyl, butyl, isobutyl, sec-butyl, tert-butyl, pentyl, isopentyl, tert-pentyl, hexyl, isohexyl, and the like. Examples of substituted alkyl groups include aryl-substituted alkyls such as benzyl.

As used herein, an alkyl optionally substituted with one or more alkyl groups includes, but is not limited to, 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and the like.

As used herein, an alkyl optionally substituted with one or more halogen groups includes, but is not limited to, CH₂F, CHF₂, CF₃, CH₂CH₂F, CH₂CHF₂, CH₂CF₃, CH₂C(CH₃)₂F, CH₂Cl, CH₂Br, and the like.

As used herein, an alkyl optionally substituted with one or more OR⁴ includes CH₂OH, CH₂CH₂OH, CH₂C(CH₃)₂OH, CH₂CH₂CH₂OH, CH₂CH₂CH(OH)CH₃, CH₂C(OH)(CH₃)₂, CH₂—O—CH₂OMe, CH₂CH₂OMe, CH₂CH₂CH₂OMe, CH₂CH₂CH(OMe)CH₃, CH₂C(OMe)(CH₃)₂, and the like.

As used herein, an alkyl optionally substituted with one or more NR⁴R⁵ includes CH₂NH₂, CH₂CH₂NH₂, CH₂CH₂CH₂NH₂, CH₂NHMe, CH₂CH₂NHMe, CH₂CH₂NHEt, CH₂CH₂NHCH₂CH₃, CH₂CH₂CH₂NHMe, CH₂NMe₂, CH₂CH₂NMe₂, CH₂CH₂CH₂NMe₂, CH₂CH₂NHCH(CH₂)₃, CH₂CH₂N(CH₂CH₃)₂, and the like.

The term “alkenyl” refers to linear or branched-chain monovalent hydrocarbon radical of two to twelve carbon atoms, containing at least one double bond, wherein the alkenyl radical may be optionally substituted independently with one or more substituents described herein, and includes radicals having “cis” and “trans” orientations, or alternatively, “E” and “Z” orientations. Examples of alkenyl groups include, but are not limited to: ethylene or vinyl (—CH═CH₂), allyl (—CH₂CH═CH₂), 1-cyclopent-1-enyl, 1-cyclopent-2-enyl, 1-cyclopent-3-enyl, 5-hexenyl (—CH₂ CH₂CH₂CH₂CH═CH₂), 1-cyclohex-1-enyl, 1-cyclohex-2-enyl, and 1-cyclohex-3-enyl.

The term “alkynyl” refers to a linear or branched monovalent hydrocarbon radical of two to twelve carbon atoms containing at least one triple bond, wherein the alkynyl radical may be optionally substituted independently with one or more substituents described herein. Examples of alkynyl groups include, but are not limited to: acetylene (—C≡CH) and propargyl (—CH₂C≡CH).

The terms “cycloalkyl,” “carbocycle” and “carbocyclyl” refer to a saturated or partially unsaturated cyclic hydrocarbon radical having three to twelve carbon atoms as a monocyclic ring or 7 to 12 carbon atoms as a bicyclic ring, wherein the cycloalkyl may be optionally substituted independently with one or more substituents described herein. Examples of cycloalkyl groups include, but are not limited to, cyclopropyl, cyclobutyl, cyclopentyl, cyclohexyl, cycloheptyl, and the like. The term “cycloalkyl” further includes bicyclic and tricyclic cycloalkyl structures, wherein the bicyclic and tricyclic structures may include a saturated or partially unsaturated cycloalkyl fused to a saturated or partially unsaturated cycloalkyl or heterocycloalkyl ring or an aryl or heteroaryl ring. Bicyclic carbocycles have 7 to 12 ring atoms, e.g., arranged as a bicyclo [4,5], [5,5], [5,6] or [6,6] system, or 9 or 10 ring atoms arranged as a bicyclo [5,6] or [6,6] system, or as bridged systems such as bicyclo[2.2.1]heptane, bicyclo[2.2.2]octane and bicyclo[3.2.2]nonane.

The term “heteroalkyl” refers to saturated linear or branched-chain monovalent hydrocarbon radical of one to twelve carbon atoms, wherein at least one of the carbon atoms is replaced with a heteroatom independently selected from N, O or S, and wherein the radical may be a carbon radical or heteroatom radical (i.e., the heteroatom may appear in the middle or at the end of the radical). The heteroalkyl radical may be optionally substituted independently with one or more substituents described herein. The term “heteroalkyl” encompasses alkoxy and heteroalkoxy radicals.

The terms “heterocycloalkyl,” “heterocycle” or “hetercyclyl” refer to a saturated or partially unsaturated carbocyclic radical of 3 to 8 ring atoms in which at least one ring atom is a heteroatom independently selected from nitrogen, oxygen and sulfur, the remaining ring atoms being C, where one or more ring atoms may be optionally substituted independently with one or more substituent described herein. The radical may be a carbon radical or heteroatom radical. The term further includes fused ring systems, which include a heterocycle fused one or more carbocyclic or heterocyclic rings. “Heterocycloalkyl” also includes radicals where heterocycle radicals are fused with aromatic or heteroaromatic rings. Examples of heterocycloalkyl rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinylimidazolinyl, imidazolidinyl, 3-azabicyco[3.1.0]hexanyl, 3-azabicyclo[4.1.0]heptanyl, azabicyclo[2.2.2]hexanyl, 3H-indolyl and quinolizinyl. Spiro moieties are also included within the scope of this definition. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl. The heterocycle groups herein are unsubstituted or, as specified, substituted in one or more substitutable positions with various groups. For example, such heterocycle groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

The term “aryl” refers to a monovalent aromatic carbocyclic radical of 6-20 carbon atoms having a single ring (e.g., phenyl), multiple rings (e.g., biphenyl), or multiple condensed rings in which at least one is aromatic, (e.g., 1,2,3,4-tetrahydronaphthyl, naphthyl), where one or more ring atoms may be optionally substituted independently with one or more substituent described herein. Some aryl groups are represented in the exemplary structures as “Ar”. Typical aryl groups include, but are not limited to, radicals derived from benzene, substituted benzene, naphthalene, anthracene, biphenyl, indenyl, indanyl, 1,2-dihydronapthalene, 1,2,3,4-tetrahydronapthyl, and the like.

The term “heteroaryl” refers to a monovalent aromatic radical of 5-, 6-, or 7-membered rings which includes fused ring systems (at least one of which is aromatic) of 5-10 atoms containing at least one and up to four heteroatoms selected from nitrogen, oxygen, or sulfur. Examples of heteroaryl groups include, but are not limited to, pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Spiro moieties are also included within the scope of this definition. Heteroaryl groups are optionally mono-, di-, or trisubstituted with, e.g., halogen, lower alkyl, lower alkoxy, haloalkyl, aryl, heteroaryl, and hydroxy.

By way of example and not limitation, carbon bonded heterocycles and heteroaryls are bonded at position 2, 3, 4, 5, or 6 of a pyridine, position 3, 4, 5 or 6 of a pyridazine, position 2, 4, 5, or 6 of a pyrimidine, position 2, 3, 5 or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7 or 8 of an isoquinoline. Examples of carbon bonded heterocycles include, but are not limited to, 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl or 5-thiazolyl.

By way of example and not limitation, nitrogen bonded heterocycles and heteroaryls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole or β-carboline. Examples of nitrogen bonded heterocycles include 1-aziridyl, 1-azetedyl, 1-pyrrolyl, 1-imidazolyl, 1-pyrazolyl and 1-piperidinyl.

The term “halogen” represents fluoro, chloro, bromo or iodo. Likewise, the term “halogen” refers to a fluorine, chlorine, bromine, or iodine substituent.

“Amino protecting groups” refers to those organic groups intended to protect nitrogen atoms against undesirable reactions during synthetic procedures and include, but are not limited to, benzyl, benzyloxycarbonyl (CBZ), tert-butoxycarbonyl (Boc), trifluoroacetyl, and the like.

“Substituted methyl”, “substituted alkyl”, “substituted aryl”, “substituted heteroaryl”, “substituted cycloalkyl” and “substituted heterocycloalkyl” mean alkyl, aryl, cycloalkyl and heterocyclyl, respectively, in which one or more hydrogen atoms are each independently replaced with a substituent. Typical substituents include, but are not limited to, F, Cl, Br, I, OH, OR, R, ═O, ═S, ═N(OR), —C(═O)R, —C(═O)OR, —C(═O)NRR′, —NRR′, —N(R)C(═O)R′, —N(R)C(═O)OR′, —N(R)C(═O)NR′R″, —SR, —OC(═O)R, —OC(═O)OR, —OC(═O)NRR′, —OS(O)₂(OR), —OP(═O)(OR)₂, —OP(OR)₂, —P(═O)(OR)₂, —S(O)R, —S(O)₂R, —S(O)₂NR, —S(O)(OR), —S(O)₂(OR), —SC(═O)R, —SC(═O)OR, ═O and —SC(═O)NRR′; where each R, R′ and R″ is independently selected from H, C₁-C₁₀ alkyl, C₁-C₁₀ alkenyl, C₁-C₁₀ alkynyl, C₆-C₂₀ aryl and C₂-C₂₀ heterocycle. Alkenyl, alkynyl, and heteroalkyl, groups as described above may also be similarly substituted.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. Accordingly, this invention also includes racemates and resolved enantiomers, and diastereomers compounds of the Formulas I. The methods for the determination of stereochemistry and the separation of stereoisomers are well known in the art (see discussion in Chapter 4 of “Advanced Organic Chemistry”, 4th edition J. March, John Wiley and Sons, New York, 1992).

In addition to compounds of the Formulas I, the invention also includes solvates, pharmaceutically acceptable prodrugs, pharmaceutically active metabolites, and pharmaceutically acceptable salts of such compounds.

The term “solvate” refers to an aggregate of a molecule with one or more solvent molecules.

A “pharmaceutically acceptable prodrug” is a compound that may be converted under physiological conditions or by solvolysis to the specified compound or to a pharmaceutically acceptable salt of such compound. Prodrugs include compounds wherein an amino acid residue, or a polypeptide chain of two or more (e.g., two, three or four) amino acid residues (i.e., peptides) is covalently joined through an amide or ester bond to a free amino, hydroxy or carboxylic acid group of compounds of the present invention. Amino acid residues include, but are not limited to, the 20 naturally occurring amino acids commonly designated by three letter symbols and also includes 4-hydroxyproline, hydroxylysine, demosine, isodemosine, 3-methylhistidine, norvaline, beta-alanine, gamma-aminobutyric acid, cirtulline, homocysteine, homoserine, ornithine and methionine sulfone. One preferred prodrug of this invention is a compound of Formula I covalently joined to a phosphate residue. Another preferred prodrug of this invention is a compound of Formula I covalently joined to a valine residue or an alanine-alanine dipeptide.

Additional types of prodrugs are also encompassed. For instance, free carboxyl groups can be derivatized as amides or alkyl esters. As another example, compounds of this invention comprising free hydroxy groups may be derivatized as prodrugs by converting the hydroxy group into groups such as, but not limited to, phosphate ester, hemisuccinate, dimethylaminoacetate, or phosphoryloxymethyloxycarbonyl groups, as outlined in Advanced Drug Delivery Reviews, 1996, 19, 115. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including, but not limited to, ether, amine and carboxylic acid functionalities, or where the acyl group is an amino acid ester as described above, are also encompassed. Prodrugs of this type are described in J. Med. Chem., 1996, 39, 10. More specific examples include replacement of the hydrogen atom of the alcohol group with a group such as (C₁-C₆)alkanoyloxymethyl, 1-((C₁-C₆)alkanoyloxy)ethyl, 1-methyl-1-((C₁-C₆)alkanoyloxy)ethyl, (C₁-C₆)alkoxycarbonyloxymethyl, N-(C₁-C₆)alkoxycarbonylaminomethyl, succinoyl, (C₁-C₆)alkanoyl, α-amino(C₁-C₄)alkanoyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate).

Free amines can also be derivatized as amides, sulfonamides or phosphonamides. All of these prodrug moieties may incorporate groups including, but not limited to, ether, amine and carboxylic acid functionalities. For example, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, or R-carbonyl is a natural α-aminoacyl or natural α-aminoacyl-natural α-aminoacyl, —C(OH)C(O)OY wherein Y is H, (C₁-C₆)alkyl or benzyl, —C(OY₀)Y₁ wherein Y₀ is (C₁-C₄) alkyl and Y₁ is (C₁-C₆)alkyl, carboxy(C₁-C₆)alkyl, amino(C₁-C₄)alkyl or mono-N— or di-N,N-(C₁-C₆)alkylaminoalkyl, —C(Y₂)Y₃ wherein Y₂ is H or methyl and Y₃ is mono-N— or di-N,N-(C₁-C₆)alkylamino, morpholino, piperidin-1-yl or pyrrolidin-1-yl.

Prodrugs of a compound may be identified using routine techniques known in the art. Various forms of prodrugs are known in the art. For examples of such prodrug derivatives, see, for example, a) Design of Prodrugs, edited by H. Bundgaard, (Elsevier, 1985) and Methods in Enzymology, Vol. 42, p. 309-396, edited by K. Widder, et al. (Academic Press, 1985); b) A Textbook of Drug Design and Development, edited by Krogsgaard-Larsen and H. Bundgaard, Chapter 5 “Design and Application of Prodrugs,” by H. Bundgaard p. 113-191 (1991); c) H. Bundgaard, Advanced Drug Delivery Reviews, 8, 1-38 (1992); d) H. Bundgaard, et al., Journal of Pharmaceutical Sciences, 77:285 (1988); and e) N. Kakeya, et al., Chem. Pharm. Bull., 32: 692 (1984), each of which is specifically incorporated herein by reference.

A “metabolite” is a pharmacologically active product produced through in vivo metabolism in the body of a specified compound or salt thereof. Such products may result for example from the oxidation, reduction, hydrolysis, amidation, deamidation, esterification, deesterification, enzymatic cleavage, and the like, of the administered compound. Accordingly, the invention includes metabolites of compounds of Formula I, including compounds produced by a process comprising contacting a compound of this invention with a mammal for a period of time sufficient to yield a metabolic product thereof.

Metabolites are typically identified by preparing a radiolabelled (e.g., ¹⁴C or ³H) isotope of a compound of the invention, administering it parenterally in a detectable dose (e.g., greater than about 0.5 mg/kg) to an animal such as rat, mouse, guinea pig, monkey, or to man, allowing sufficient time for metabolism to occur (typically about 30 seconds to 30 hours) and isolating its conversion products from the urine, blood or other biological samples. These products are easily isolated since they are labeled (others are isolated by the use of antibodies capable of binding epitopes surviving in the metabolite). The metabolite structures are determined in conventional fashion, e.g., by MS, LC/MS or NMR analysis. In general, analysis of metabolites is done in the same way as conventional drug metabolism studies well known to those skilled in the art. The metabolites, so long as they are not otherwise found in vivo, are useful in diagnostic assays for therapeutic dosing of the compounds of the invention.

A “pharmaceutically acceptable salt” is a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable sale. Examples of pharmaceutically acceptable salts include those salts prepared by reaction of the compounds of the present invention with a mineral or organic acid or an inorganic base, such salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, flimarates, maleates, butyn-1,4-dioates, hexyne-1,6-dioates, benzoates, chlorobenzoates, methylbenzoates, dinitromenzoates, hydroxybenzoates, methoxybenzoates, phthalates, sulfonates, xylenesulfonates, pheylacetates, phenylpropionates, phenylbutyrates, citrates, lactates, γ-hydroxybutyrates, glycollates, tartrates, methanesulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alphahydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include, but are not limited to, organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

The compounds of Formula I also include other salts of such compounds which are not necessarily pharmaceutically acceptable salts, and which may be useful as intermediates for preparing and/or purifying compounds of Formula I and/or for separating enantiomers of compounds of Formula I.

The inventive compounds may be prepared using the reaction routes and synthesis schemes as described below, employing the techniques available in the art using starting materials that are readily available.

FIGS. 1 and 2 show examples of synthetic routes for the preparation of compounds of Formula I.

Methods Of Separation

In each of the exemplary Schemes it may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like.

Selection of appropriate methods of separation depends on the nature of the materials involved. For example, boiling point and molecular weight in distillation and sublimation, presence or absence of polar functional groups in chromatography, stability of materials in acidic and basic media in multiphase extraction, and the like. One skilled in the art will apply techniques most likely to achieve the desired separation.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Also, some of the compounds of the present invention may be atropisomers (e.g., substituted biaryls) and are considered as part of this invention. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994; Lochmuller, C. H., (1975) J. Chromatogr., 113(3):283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

Under separation method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by separation method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III. (1982) J. Org. Chem. 47:4165), of the racemic mixture, and analyzing the NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (“Chiral Liquid Chromatography” (1989) W. J. Lough, Ed., Chapman and Hall, New York; Okamoto, (1990) J. of Chromatogr. 513:375-378). Enriched or purified enantiomers can be distinguished by methods used to distinguish other chiral molecules with asymmetric carbon atoms, such as optical rotation and circular dichroism.

Administration of Compounds of Formula I

Therapeutically effective amounts of the compounds of the invention may be used to treat diseases mediated by modulation or regulation of protein kinases. An “effective amount” is intended to mean that amount of compound that, when administered to a mammal in need of such treatment, is sufficient to effect treatment for a disease mediated by the activity of one or more protein kinases, such as that p38 alpha and the associated p38 mediated events such as cytokine production. Thus, for example, a therapeutically effective amount of a compound selected from Formula I or a salt, active metabolite or prodrug thereof, is a quantity sufficient to modulate, regulate, or inhibit the activity of one or more protein kinases such that a disease condition which is mediated by that activity is reduced or alleviated.

The compounds of the invention may be administered by any route appropriate to the condition to be treated. Suitable routes include oral, parenteral (including subcutaneous, intramuscular, intravenous, intraarterial, intradermal, intrathecal and epidural), intraocular, transdermal, rectal, nasal, topical (including buccal and sublingual), vaginal, intraperitoneal, intrapulmonary and intranasal. For local immunosuppressive treatment, the compounds may be administered by intralesional administration, including perfusing or otherwise contacting the graft with the inhibitor before transplantation. It will be appreciated that the preferred route may vary with for example the condition of the recipient. Where the compound is administered orally, it may be formulated as a pill, capsule, tablet, etc. with a pharmaceutically acceptable carrier or excipient. Where the compound is administered parenterally, it may be formulated with a pharmaceutically acceptable parenteral vehicle and in a unit dosage injectable form, as detailed below.

The amount of a given agent that will correspond to such an amount will vary depending upon factors such as the particular compound, disease condition and its severity, the identity (e.g., weight) of the mammal in need of treatment, but can nevertheless be routinely determined by one skilled in the art. “Treating” is intended to mean at least the mitigation of a disease condition in a mammal, such as a human, that is affected, at least in part, by the activity of one or more protein kinases, such as p38, and includes, but is not limited to, preventing the disease condition from occurring in a mammal, particularly when the mammal is found to be predisposed to having the disease condition but has not yet been diagnosed as having it; modulating and/or inhibiting the disease condition; and/or alleviating the disease condition.

Pharmaceutical Formulations

In order to use a compound of the Formula I, or a pharmaceutically acceptable salt or in vivo cleavable prodrug thereof, for the therapeutic treatment (including prophylactic treatment) of mammals including humans, it is normally formulated in accordance with standard pharmaceutical practice as a pharmaceutical composition. According to this aspect of the invention there is provided a pharmaceutical composition that comprises a compound of the Formula I, or a pharmaceutically acceptable salt or in vivo cleavable prodrug thereof, as defined hereinbefore in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, or intramuscular dosing or as a suppository for rectal dosing). For example, compositions intended for oral use may contain, for example, one or more coloring, sweetening, flavoring and/or preservative agents.

Suitable pharmaceutically-acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose. methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), coloring agents, flavoring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavoring and coloring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavoring and preservative agents.

Syrups and elixirs may be formulated with sweetening agents such as glycerol, propylene glycol, sorbitol, aspartame or sucrose, and may also contain a demulcent, preservative, flavoring and/or coloring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedures well known in the art.

Compositions for administration by insufflation may be in the form of a finely divided powder containing particles of average diameter of, for example, 30 μm or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurized aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

Other means of systemic administration which may utilize the active ingredients of the present invention in either liquid or solid form include transdermal, intranasal, and opthalmic routes. In particular, transdermal patches prepared in accordance with well known drug delivery technology may be prepared and applied to the skin of a patient to be treated. The active agent by reason of its formulated solubility characteristics migrates across the epidermis and into the dermal layers of the patient's skin where it is taken up as part of the general circulation of the patient, ultimately providing systemic distribution of the active ingredient over a desired, extended period of time. Also included are implants which are placed beneath the epidermal layer of the skin, i.e. between the epidermis and the dermis of the skin of the patient being treated. Such an implant will be formulated in accordance with well known principles and materials commonly used in this delivery technology, and may be prepared in such a way as to provide controlled-, sustained-, and/or delayed-release of the active ingredient into the systemic circulation of the patient. Such subepidermal (subcuticular) implants provide the same facility of installation and delivery efficiency as transdermal patches, but without the limitation of being subject to degradation, damage or accidental removal as a consequence of being exposed on the top layer of the patient's skin.

Pharmaceutical compositions of this invention may also be delivered using a drug delivery device such as an implant. Such implants may be biodegradable and/or biocompatible implants, or may be non-biodegradable implants. The implants may be permeable or impermeable to the active agent. Ophthalmic drug delivery devices may be inserted into a chamber of the eye, such as the anterior or posterior chambers or may be implanted in or on the sclera, choroidal space, or an avascularized region exterior to the vitreous. In one embodiment, the implant may be positioned over an avascular region, such as on the sclera, so as to allow for transcleral diffusion of the drug to the desired site of treatment, e.g., the intraocular space and macula of the eye. Furthermore, the site of transcleral diffusion may be proximity to a site of neovascularization such as a site proximal to the macula.

For further information on formulations, see Chapter 25.2 in Volume 5 of Comprehensive Medicinal Chemistry (Corwin Hansch; Chairman of Editorial Board), Pergamon Press 1990, which is specifically incorporated herein by reference.

The amount of a compound of this invention that is combined with one or more excipients to produce a single dosage form will necessarily vary depending upon the host treated and the particular route of administration. For example, a formulation intended for oral administration to humans may contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on routes of administration and dosage regimes, see Chapter 25.3 in Volume 5 of Comprehensive Medicinal Chemistry (Corwin Hansch; Chairman of Editorial Board), Pergamon Press 1990, which is specifically incorporated herein by reference.

The size of the dose for therapeutic or prophylactic purposes of a compound of Formula I will naturally vary according to the nature and severity of the conditions, the age and sex of the animal or patient and the route of administration, according to well known principles of medicine.

In one aspect of this invention, the compounds of this invention or pharmaceutical salts or prodrugs thereof may be formulated into pharmaceutical compositions for administration to animals or humans to treat or prevent a p38-mediated condition. The term “p38-mediated condition” as used herein means any disease or other deleterious condition in which p38 is known to play a role. This includes conditions which are known to be caused by IL-1, TNF, IL-6 or IL-8 overproduction. Such conditions include, without limitation, inflammatory diseases, autoimmune diseases, destructive bone disorders, hyperproliferative disorders, infectious diseases, viral disease, and neurodegenerative diseases

Inflammatory diseases which may be treated or prevented include, but are not limited to, acute pancreatitis, chronic pancreatitis, asthma, allergies, and adult respiratory distress syndrome.

Autoimmune diseases which may be treated or prevented include, but are not limited to, glomeralonephritis, rheumatoid arthritis, systemic lupus erythematosus, scleroderma, chronic thyroiditis, Graves' disease, autoimmune gastritis, insulin-dependent diabetes mellitus (Type I), autoimmune hemolytic anemia, autoimmune neutropenia, thrombocytopenia, atopic dermatitis, chronic active hepatitis, myasthenia gravis, multiple sclerosis, inflammatory bowel disease, ulcerative colitis, Crohn's disease, psoriasis, or graft vs. host disease.

Destructive bone disorders which may be treated or prevented include, but are not limited to, osteoporosis, osteoarthritis and multiple myeloma-related bone disorder.

Hyperproliferative diseases which may be treated or prevented include, but are not limited to, cancer such as brain, lung, squamous cell, bladder, gastric, pancreatic, breast, head, neck, renal, kidney, ovarian, prostate, colorectal, esophageal, testicular, gynecological or thyroid cancer. In another embodiment, said method relates to the treatment of a non-cancerous hyperproliferative disorder such as benign hyperplasia of the skin (e.g., psoriasis), restenosis, or prostate (e.g., benign prostatic hypertrophy (BPH), acute myelogenous leukemia, chronic myelogenous leukemia, metastatic melanoma, Kaposi's sarcoma, and multiple myeloma.

Infectious diseases which may be treated or prevented include, but are not limited to, sepsis, septic shock, and Shigellosis.

Viral diseases which may be treated or prevented include, but are not limited to, acute hepatitis infection (including hepatitis A, hepatitis B and hepatitis C), HIV infection and CMV retinitis.

Degenerative conditions or diseases which may be treated or prevented by the compounds of this invention include, but are not limited to, Alzheimer's disease, Parkinson's disease, cerebral ischemia and other neurodegenerative diseases.

“p38-mediated conditions” also include ischemia/reperfusion in stroke, heart attacks, myocardial ischemia, organ hypoxia, vascular hyperplasia, cardiac hypertrophy and thrombin-induced platelet aggregation.

In addition, the p38 inhibitors of this invention are also capable of inhibiting the expression of inducible pro-inflammatory proteins such as prostaglandin endoperoxide synthase-2 (PGHS-2), also referred to as cyclooxygenase-2 (COX-2). Therefore, other “p38-mediated conditions” are edema, analgesia, fever and pain, such as neuromuscular pain, headache, cancer pain, dental pain and arthritis pain.

The conditions and diseases that may be treated or prevented by the p38 inhibitors of this invention may also be conveniently grouped by the cytokine (e.g., IL-1, TNF, IL-6, IL-8) that is believed to be responsible for the disease.

Thus, an IL-1-mediated disease or condition includes rheumatoid arthritis, osteoarthritis, stroke, endotoxemia and/or toxic shock syndrome, inflammatory reaction induced by endotoxin, inflammatory bowel disease, tuberculosis, atherosclerosis, muscle degeneration, cachexia, psoriatic arthritis, Reiter's syndrome, gout, traumatic arthritis, rubella arthritis, acute synovitis, diabetes, pancreatic β-cell disease and Alzheimer's disease.

A TNF-mediated disease or condition includes rheumatoid arthritis, rheumatoid spondylitis, osteoarthritis, gouty arthritis and other arthritic conditions, sepsis, septic shock, endotoxic shock, gram negative sepsis, toxic shock syndrome, adult respiratory distress syndrome, cerebral malaria, chronic pulmonary inflammatory disease, silicosis, pulmonary sarcoisosis, bone resorption diseases, reperfusion injury, graft vs. host reaction, allograft rejections, fever and myalgias due to infection, cachexia secondary to infection, cachexia secondary to severe diseases (such as, but not limited to, cancer, acquired immunodeficiency syndrome or chronic failure), cachexia secondary to treatment with other therapeutic agents (such as, but not limited to, cancer chemotherapeutics and immunosuppressants), cachexia from conditions that cause the production and/or release of inflammatory cytokines such as IL1-alpha, TNF alpha and IL6, cachexia from non-specific or idiopathic causes, ARC or malignancy, keloid formation, scar tissue formation, Crohn's disease, ulcerative colitis or pyresis.

TNF-mediated diseases also include fatigue secondary to severe diseases (such as, but not limited to, cancer, acquired immunodeficiency syndrome or chronic failure), fatigue secondary to treatment with other therapeutic agents (such as, but not limited to, cancer chemotherapeutics and immunosuppressants), and fatigue from conditions that cause the production and/or release of inflammatory cytokines such as IL1-alpha, TNF alpha and IL6, cachexia from non-specific or idiopathic causes.

TNF-mediated diseases also include viral infections, such as HIV, CMV, influenza and herpes; and veterinary viral infections, such as lentivirus infections, including, but not limited to equine infectious anemia virus, caprine arthritis virus, visna virus or maedi virus; or retrovirus infections, including feline immunodeficiency virus, bovine immunodeficiency virus, or canine immunodeficiency virus.

IL-8 mediated disease or condition includes diseases characterized by massive neutrophil infiltration, such as psoriasis, inflammatory bowel disease, asthma, cardiac and renal reperfusion injury, adult respiratory distress syndrome, thrombosis and glomerulonephritis.

In addition, the compounds of this invention may be used topically to treat or prevent conditions caused or exacerbated by IL-1 or TNF. Such conditions include inflamed joints, eczema, psoriasis, inflammatory skin conditions such as sunburn, inflammatory eye conditions such as conjunctivitis, pyresis, pain and other conditions associated with inflammation.

In addition, the compounds of this invention may be used to treat feelings of general malaise secondary to severe diseases (such as, but not limited to, cancer, acquired immunodeficiency syndrome or chronic failure), fatigue secondary to treatment with other therapeutic agents (such as, but not limited to, cancer chemotherapeutics and immunosuppressants), and from conditions that cause the production and/or release of inflammatory cytokines such as IL1-alpha, TNF alpha and IL6, cachexia from non-specific or idiopathic causes.

The compounds of this invention may be used in combination with other drugs and therapies used in the treatment of disease states which would benefit from the inhibition of cytokines, in particular IL-1, TNF, IL-6 or IL-8. The dose of the second drug can be appropriately selected based on a clinically employed dose. The proportion of the compound of the present invention and the second drug can be appropriately determined according to the administration subject, the administration route, the target disease, the clinical condition, the combination, and other factors. In cases where the administration subject is a human, for instance, the second drug may be used in an amount of 0.01 to 100 parts by weight per part by weight of the compound of the present invention.

The second compound of the pharmaceutical combination formulation or dosing regimen preferably has complementary activities to the compound of this invention such that they do not adversely affect each other. Such drugs are suitably present in combination in amounts that are effective for the purpose intended. Accordingly, another aspect of the present invention provides a composition comprising a compound of this invention in combination with a second drug, such as described herein.

A compound of this invention and the additional pharmaceutically active agent(s) may be administered together in a unitary pharmaceutical composition or separately and, when administered separately this may occur simultaneously or sequentially in any order. Such sequential administration may be close in time or remote in time. The amounts of the compound of this invention and the second agent(s) and the relative timings of administration will be selected in order to achieve the desired combined therapeutic effect. Alternatively, the additional pharmaceutically active agent can be administered intermittently as needed.

The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

For example, by virtue of their ability to inhibit cytokines, the compounds of Formula I are of value in the treatment of certain inflammatory and non-inflammatory diseases which are currently treated with a cyclooxygenase-inhibitory non-steroidal anti-inflammatory drug (NSAID) such as indomethacin ketorolac, acetylsalicylic acid, ibuprofen, sulindac, tolmetin and piroxicam. Co-administration of a compound of the Formula I with a NSAID can result in a reduction of the quantity of the latter agent needed to produce a therapeutic effect, and thus the likelihood of adverse side-effects from the NSAID such as gastrointestinal effects are reduced. Thus according to a further feature of the invention there is provided a pharmaceutical composition which comprises a compound of Formula I, or a pharmaceutically-acceptable salt or in vivo cleavable prodrug thereof, in conjunction or admixture with a cyclooxygenase inhibitory non-steroidal anti-inflammatory agent, and a pharmaceutically-acceptable diluent or carrier.

The compounds of Formula I may also be used in the treatment of conditions such as rheumatoid arthritis in combination with anti-arthritic agents such as gold, methotrexate, steroids and penicillinamine, and in conditions such as osteoarthritis in combination with steroids.

The compounds of the present invention may also be used in the treatment of degradative diseases, for example osteoarthritis, with chondroprotective, anti-degradative and/or reparative agents such as Diacerhein, hyaluronic acid formulations such as Hyalan, Rumalon, Arteparon and glucosamine salts such as Antril.

The compounds of Formula I may also be used in the treatment of asthma in combination with anti-asthmatic agents such as bronchodilators and leukotriene antagonists.

The compounds of Formula I may also be used as an adjunct to therapies with drugs such as chemotherapeutics and immunosuppressants, in order to reduce drug-induced side effects and/or increase drug regime compliance, and/or allow the use of otherwise non-tolerated drugs or drug combinations in patients, and/or expand the duration of drug treatment.

Kits

In another embodiment of the invention, an article of manufacture, or “kit”, containing materials useful for the treatment of the disorders described above is provided. In one embodiment, the kit comprises a container comprising a compound of Formula I or a formulation thereof. The kit may also comprise a label or package insert on or associated with the container. The term “package insert” is used to refer to instructions customarily included in commercial packages of therapeutic products, that contain information about the indications, usage, dosage, administration, contraindications and/or warnings concerning the use of such therapeutic products. Suitable containers include, for example, bottles, vials, syringes, blister pack, etc. The container may be formed from a variety of materials such as glass or plastic. The container holds a compound of Formula I or a formulation thereof which is effective for treating the condition and may have a sterile access port (for example, the container may be an intravenous solution bag or a vial having a stopper pierceable by a hypodermic injection needle). The label or package insert may indicate that the composition is used for treating the condition of choice, such as cancer. In one embodiment, the label or package inserts indicates that the compound of Formula I or a formulation thereof can be used to treat a p38-mediated conditions. In addition, the label or package insert may indicate that the patient to be treated is one having a p38-mediated condition such as inflammatory disease, autoimmune disease, destructive bone disorder, proliferative disorder, infectious disease, viral disease, and neurodegenerative disease. The label or package insert may also indicate that the composition can be used to treat other disorders. Alternatively, or additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

According to another embodiment, a kit may comprise (a) a first container with a compound of Formula I or a formulation thereof contained therein; and optionally (b) a second container with a second pharmaceutical formulation contained therein, wherein the second pharmaceutical formulation comprises a second compound with anti-inflammatory activity. Alternatively, or additionally, the article of manufacture may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of a compound of Formula I or a formulation thereof and, if present, the second pharmaceutical formulation. For example, if the kit comprises a compound of Formula I or a formulation thereof (“first formulation”) and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of a compound of Formula I, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

In certain other embodiments wherein the kit comprises a compound of Formula I or a formulation thereof and a second therapeutic agent, the kit may comprise a container for containing the separate components such as a divided bottle or a divided foil packet, however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

Accordingly this invention also provides a kit for treating an abnormal cell growth condition, wherein said kit comprises a) a first pharmaceutical composition comprising a compound of this invention or a pharmaceutically acceptable salt thereof; and b) instructions for use.

In certain embodiments, the kit further comprises (c) a second pharmaceutical composition, wherein the second pharmaceutical composition comprises a second compound having anti-hyperproliferative activity. In certain embodiment comprising a second pharmaceutical composition, the kit further comprises instructions for the simultaneous, sequential or separate administration of said first and second pharmaceutical compositions to a patient in need thereof. In certain embodiments, said first and second pharmaceutical compositions are contained in separate containers. In other embodiments, said first and second pharmaceutical compositions are contained in the same container.

Although the compounds of Formula I are primarily of value as therapeutic agents for use in warm-blooded animals (including man), they are also useful whenever it is required to inhibit the effects of cytokines. Thus, they are useful as pharmacological standards for use in the development of new biological tests and in the search for new pharmacological agents.

The activity of the compounds of this invention may be assayed for p38 inhibition in vitro, in vivo, or in a cell line. In vitro assays include assays that determine inhibition of either the kinase activity or ATPase activity of activated p38. Alternate in vitro assays quantitate the ability of the inhibitor to bind to p38 and may be measured either by radiolabelling the inhibitor prior to binding, isolating the inhibitor/p38 complex and determining the amount of radiolabel bound, or by running a competition experiment where new inhibitors are incubated with p38 bound to known radioligands. These and other useful in vitro and cell culture assays are well known to those of skill in the art.

Cell culture assays of the inhibitory effect of the compounds of this invention may be used to determine the amounts of TNF-α, IL-1, IL-6 or IL-8 produced in whole blood or cell fractions thereof in cells treated with inhibitor as compared to cells treated with negative controls. Level of these cytokines may be determined through the use of commercially available ELISAs or as described in the Biological Examples section below.

### BIOLOGICAL EXAMPLES

The biological activities of the compounds of the invention were demonstrated by the following in vitro assays.

### Example A

### p38 Biochemical Assay

P38 activity was assayed at room temperature in a 100 μL reaction containing 5 nM activated p38α enzyme and 1 uM ATF-2 (Activating Transcription Factor 2 fusion protein) as the substrate in 25 mM HEPES (pH 7.4), 100 μM Vanadate, 1 mM DTT, 10 mM MgCl₂ and 10 μM [γ-³³P]-ATP (˜0.1 μCi P³³/reaction). The reaction was terminated after 30-40 minutes by adding 25% TCA, let stand for 5 minutes and then transferred directly to a GF-B membrane filter plate. The filter was washed twice for 30 seconds with 0.5% phosphoric acid using a Tomtec Mach III Automated Harvestor. After washing, the vacuum was continued for 30 seconds to dry the filter. Approximately 30 μL of scintillant was added per well to the filter plate and then read in a Liquid Scintillation Counter (Packard TopCount HTS).

### Example B

### Phospho-HSP27 (Ser78) LICOR Odyssey Cell-Based Assay Protocol

This assay is used to measure the ability of various compounds to inhibit the p38 pathway in anisomycin-stimulated HeLa cells.

HeLa cells were plated out at 10,000 cells/well of a 96-well, black, clear-bottomed, standard tissue culture treated plate 24 hours prior to compound treatment. Cells were treated with varying concentrations of compound, according to well, maintaining a constant concentration of DMSO (1% final), and incubated with compound for 60 minutes at 37° C., 5% CO₂. Cells were then stimulated with 1 μg/mL final concentration of Anisomycin and incubated for an additional 60 minutes at 37° C., 5% CO₂. Medium above the cells was vigorously poured off and each well was washed 1×150 μL PBS. Plates were fixed in 150 μL/well 3.7% formaldehyde/PBS for 15 minutes at room temperature, washed 5×150 μL PBS/well, and extracted for 15 minutes with 150 μL/well −20° C. MeOH. Cells were rehydrated by washing wells 3×150 μL PBS for 5′ each. Plates were blocked for overnight at 4° C. in Odyssey blocking buffer (LI-COR, catalog #927-40000), and then incubated with 50 μL/well primary antibody solution for 2 hours at room temperature.

For the primary antibody solution, primary antibodies were diluted in Odyssey blocking buffer supplemented with 0.1% Tween-20 as follows:

| Antibody | Host Species | Supplier | Catalog # | Dilution |
|---|---|---|---|---|
| pHSP27 | rabbit pAb | Stressgen | 2401 | 1:2000 |
| (Ser78) | | | | |
| GAPDH | mouse mAb | R&D Systems | RDI-TRK5-6C5 | 1 μg/mL |

Plates were washed with PBS/0.1% Tween-20 in a platewasher (5×300 μL/well), and then incubated with secondary antibody solution for 60 minutes at room temperature. Secondary antibodies were diluted in Odyssey blocking buffer supplemented with 0.1% Tween-20 as follows: 1:1000 dilution Alexa680 conjugated goat-anti-rabbit IgG (Molecular Probes, Cat#: A21109); 1:1000 dilution of IR800DyeCW-conjugated goat-anti-mouse IgG (Rockland Immunochemicals, Inc., Cat#: 610-131-121).

Plates were washed with PBS/0.1% Tween-20 in a platewasher (5×300 μL/well), and then each well was supplemented with 100 μL PBS and imaged and quantified on the LI-COR Odyssey imager. Phospho-HSP27 values associated with each of the wells was normalized to the GAPDH signal for that well as a normalization control. These values were converted to percent of induced control (POC) by the following formula:
[Well-Assay Minimum]/[Assay Maximum]×100

where Assay minimum=average of replicate wells treated only with 1% DMSO and no anisomycin, and Assay Maximum=average of replicate wells treated with 1% DMSO and 1 μg/mL anisomycin.

The POC values were plotted out as a function of compound concentration. A 4-parameter curve fitting algorithm was applied to generate a dose response curve and an associated IC₅₀ value for the compound.

### Example C

### Human Whole Blood TNF-α Assay

Compound test solutions were made by making 3.33 fold serial dilutions in DMSO, which dilutions were then diluted to 5× stocks by diluting with MEM, 2% heat inactivated fetal bovine serum (“FBS”), 20 mM HEPES, 2 mM L-glutamine, and 1% penicillin/streptomycin.

Whole blood was collected from human volunteers using sodium heparin Vacutainer™ tubes and processed within two hours of collection. Blood was diluted 3-fold with Whole Blood (WB) medium (RPMI 1640, 2% heat inactivated fetal bovine serum, 20 mM HEPES, 2 mM L-glutamine, and 1% penicillin/streptomycin). 100 μL of diluted blood was added to each well of a 96-well cell culture plate, followed by 30 μL of a compound test solution.

After a one-hour incubation at 37° C./5% CO₂, 20 μL of 7.5 ng/mL lipopolysaccharide (E. coli K-235, Sigma L2018) was added to each well. The cells were incubated again at 37° C./5% CO₂ for 16-20 hours. The test compound supernatants were collected and assayed for TNF-α content by ELISA methods. Briefly, test compound supernatants were added to wells of a 96-well plate that were coated with antibody to human TNF-α (R&D Systems, MAB210) and incubated at room temperature for at least one hour. After washing with wash buffer, wells were incubated at room temperature with 100 μL of 0.2 μg/mL biotinylated goat anti-human TNF-α (R&D Systems, BAF210) in “antibody diluent” (20 mM HEPES, pH 7.4, 150 rAM NaCl, 2 mM MgCl₂, 1% BSA, 0.02% Tween-20) for another hour. After washing, the plate was incubated with 100 μL of 0.02 μg/mL streptavidin-alkaline phosphatase in antibody diluent for an additional hour. 200 μL of the colorimetric substrate p-nitrophenyl phosphate (pNPP, 1 mg/mL) in diethanolamine buffer with 0.5 mM MgCl₂ was added to each well. After incubation at room temperature for 30-40 minutes, the reaction was stopped by the addition of 2N NaOH. The absorbance at 405 nm was then read.

### Example D

### Mouse Model of LPS-Induced TNF-α Production

TNF-α was induced in male DBA-2J mice (from Jackson Laboratories) by tail vein injection with 2 mg/kg lipopolysaccharide (from Sigma, St. Louis). Ninety minutes later isoflurane anaesthetized mice were bled by cardiac puncture. The blood samples were then allowed to clot for two hours at 4° C. and centrifuged. Serum was separated into eppendorf tubes for later TNF-α analysis. TNF-α analysis was performed using an ELISA kit (Quantikine, Minn.) and was performed according to the instructions that accompanied the kit.

### Example E

### Plasma and Whole Blood Stability

The plasma stability of certain compounds of Formula I in blood and plasma samples (obtained from rats, monkeys and humans) was determined by measuring the rate of formation of the acid (i.e., where Y is COOH) corresponding to the tested compound of Formula I. Acid levels were measured at various time points by HPLC and compared with a synthesized acid standard. From these data, the half-life to the compound of Formula I in blood and plasma was calculated. Those compounds that demonstrated lowered or minimal formation of the corresponding acid were deemed esterase/amidase stable in the respective species.

### Preparative Examples

In order to illustrate the invention, the following examples are included. However, it is to be understood that these examples do not limit the invention and are only meant to suggest a method of practicing the invention. Persons skilled in the art will recognize that the chemical reactions described may be readily adapted to prepare a number of other p38 inhibitors of the invention, and alternative methods for preparing the compounds of this invention are deemed to be within the scope of this invention. For example, the synthesis of non-exemplified compounds according to the invention may be successfully performed by modifications apparent to those skilled in the art, e.g., by appropriately protecting interfering groups, by utilizing other suitable reagents known in the art other than those described, and/or by making routine modifications of reaction conditions. Alternatively, other reactions disclosed herein or known in the art will be recognized as having applicability for preparing other compounds of the invention.

### Examples

In the examples described below, unless otherwise indicated all temperatures are set forth in degrees Celsius. Reagents were purchased from commercial suppliers such as Aldrich Chemical Company, Lancaster, TCI or Maybridge, and were used without further purification unless otherwise indicated. Tetrahydrofuran (THF), N,N-dimethylformamide (DMF), dichloromethane (DCM), toluene, dioxane and 1,2-difluoroethane were purchased from Aldrich in Sure seal bottles and used as received.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography was done on a Biotage system (Manufacturer: Dyax Corporation) having a silica gel column or on a silica SepPak cartridge (Waters).

¹H-NMR spectra were recorded on a Varian instrument operating at 400 MHz.

¹H-NMR spectra were obtained as CDCl₃ solutions (reported in ppm), using chloroform as the reference standard (7.25 ppm). Other NMR solvents were used as needed. When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

Compounds 9a-9c were prepared as shown in FIG. 1 and as described in Examples 1-3 below.

### Example 1

### (S)-Methyl 2-(5-(2,4-difluorophenoxy)-1-isobutyl-1H-indazole-6-carboxamido)-4-(dimethylamino)-2-methylbutanoate (9a)

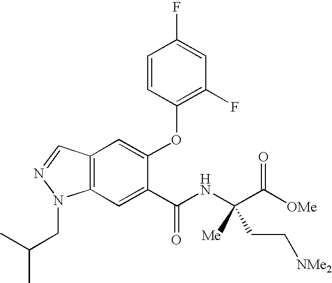

Step A: Preparation of (R)-2-isopropyl-3,6-dimethoxy-2,5-dihydropyrazine (1): To a 2 L round-bottomed flask were added (R)-3-isopropylpiperazine-2,5-dione (20.7 g, 133 mmol), Me₃OBF₄ (49.0 g, 331 mmol) and CH₂Cl₂ (500 mL). The slurry was stirred vigorously at room temperature under nitrogen atmosphere. After stirring 18 hours, the slurry became a clear solution with very viscous yellow oil settling on the bottom of the flask. An additional equivalent of Me₃OBF₄ (19.6 g, 133 mmol) was added and the mixture was stirred at room temperature. After 23 hours, the mixture was cooled in an ice bath, and 200 g of ice and 100 mL of concentrated ammonium hydroxide solution (28%) were added to the reaction mixture. The reaction mixture was stirred in an ice bath for 1 hour. The layers were separated and aqueous layer was extracted with CH₂Cl₂ (2×50 mL). The combined organic layers were washed with saturated NaHCO₃ solution (2×100 mL) and brine (100 mL), dried over K₂CO₃, filtered through a Celite pad, and concentrated under reduced pressure to provide 25.9 g of light brown oil. The crude material was purified by chromatography with 1:4 ether/pentane to provide 17.464 g of compound 1 as a colorless oil (71.5% yield). ¹H NMR (400 MHz, CDCl₃) δ 4.08-3.94 (m, 3H), 2.95 (s, 3H), 2.87 (s, 3H), 2.30-2.18 (m, 1H), 1.04 (d, J=7.03 Hz, 3H), 0.76 (d, J=6.64 Hz, 3H).

Step B: Preparation of (2R)-2-isopropyl-3,6-dimethoxy-5-methyl-2,5-dihydropyrazine (2a): To a flame-dried 250 mL round-bottomed flask was added 1 (10.04 g, 54.495 mmol) in THF (100 mL). The mixture was cooled to −78° C. and stirred for 0.5 hours. n-Butyl lithium (2.5 M in hexanes; 23.978 mL, 59.945 mmol) was slowly added at −78° C. and the mixture was stirred for 1.5 hours at −78° C. Methyl iodide (6.7851 mL; 2.50 M in hexanes, 108.99 mmol) was added. The mixture was stirred at −78° C. for 1 hour and then placed in a freezer (−18° C.) for 18 hours. The mixture was warmed to room temperature and quenched with saturated NaHCO₃ solution (70 mL). The layers were separated, and the aqueous layer was diluted with water (30 mL) and extracted with CH₂Cl₂ (3×30 mL). The combined organic layers were washed with brine (2×50 mL), dried over MgSO₄, filtered through a Celite pad, and concentrated under reduced pressure to provide 11.6 g of oil. The crude oil was purified by chromatography with 1:9 ether/hexanes. Fraction #2 and Fraction #3 were combined to provide 8.841 g of 2a as a colorless oil (2:3 ratio; 81.8% yield). Fraction #2: ¹H NMR (400 MHz, CDCl₃) δ 4.04-3.98 (m, 1H), 3.98-3.94 (m, 1H), 3.70 (s, 3H), 3.68 (s, 3H), 2.32-2.20 (m, 1H), 1.36 (d, J=7.03 Hz, 3H), 1.05 (d, J=7.03 Hz, 3H), 0.71 (d, J=7.03 Hz, 3H). Fraction #3: ¹H NMR (400 MHz, CDCl₃) δ 4.11-4.04 (m, 1H), 3.98-3.95 (m, 1H), 3.69 (s, 3H), 3.67 (s, 3H), 2.26-2.14 (m, 1H), 1.38 (d, J=7.03 Hz, 3H), 1.07 (d, J=7.03 Hz, 3H), 0.75 (d, J=7.03 Hz, 3H).

Step C: Preparation of 2-((2S,5R)-5-isopropyl-3,6-dimethoxy-2-methyl-2,5-dihydropyrazin-2-yl)ethanol (3a): Compound 2a (8.841 g, 44.59 mmol) and THF (90 mL) were added to a flask and the reaction mixture was cooled to −78° C. After 0.5 hours n-butyl lithium (2.50 M in hexanes, 41.03 mL, 102.6 mmol) was added at −78° C. After 1.5 hours at −78° C., 2-bromoethanol (3.477 mL, 49.05 mmol) was added. The mixture was stirred at −78° C. for 2 hours and then slowly warmed to −50° C. The bath temperature was maintained at −50±5° C. for 1 hour and then the mixture was placed in a freezer (−18° C.) for 2.5 days. The mixture was warmed to 0° C. and quenched with saturated NaHCO₃ solution (100 mL). The layers were separated and the aqueous layer was extracted with CH₂Cl₂ (3×10 mL). The combined organic extracts were washed with brine (10 mL), dried over MgSO₄, filtered through a Celite pad, and concentrated under reduced pressure to provide 11.8 g of an oil. The crude oil was purified by chromatography with 1:1 ether/hexanes to provide 7.774 g of 3a as oil (72% yield). ¹H NMR (400 MHz, CDCl₃) δ 3.99 (d, J=3.51 Hz, 1H), 3.98-3.95 (m, 1H), 3.81-3.76 (m, 1H), 3.67 (s, 3H), 3.65 (s, 3H), 2.29-2.21 (m, 1H), 2.03-1.90 (m, 2H), 1.40 (s, 3H), 1.08 (d, J=7.03 Hz, 3H), 0.71 (d, J=6.64 Hz, 3H).

Step D: Preparation of (2S,5R)-2-(2-bromoethyl)-5-isopropyl-3,6-dimethoxy-2-methyl-2,5-dihydropyrazine (4a): To a round-bottomed flask were placed 3a (7.774 g, 32.08 mmol), CBr₄ (12.77 g, 38.50 mmol) and CH₂Cl₂ (50 mL). The mixture was cooled in an ice bath and stirred for 15 minutes. PPh₃ (12.62 g, 48.12 mmol) was added portionwise at 0° C. The the ice bath was removed after 40 minutes and the reaction mixture was allowed to warm to room temperature. After 2 hours the reaction mixture was concentrated under reduced pressure at room temperature. Ether (50 mL) was added to the residue and a solid fell out of solution. The mixture was stirred vigorously for 0.5 hours and filtered through a medium frit funnel. The filtrate was concentrated under reduced pressure and purified by chromatography with 1:19 ether/hexanes to provide 8.134 g of 4a as an oil (83.1% yield) after drying overnight under high vacuum. ¹H NMR (400 MHz, CDCl₃) δ 3.95 (d, J=3.51 Hz, 1H), 3.68 (s, 3H), 3.66 (s, 3H), 3.21-3.15 (m, 1H), 3.13-3.06 (m, 1H), 2.43-2.34 (m, 1H), 2.29-2.21 (m, 1H), 2.17-2.09 (m, 1 h), 1.34 (s, 3H), 1.07 (d, J=7.03 Hz, 3H), 0.69 (d, J=7.03 Hz, 3H).

Step E: Preparation of 2-((2S,5R)-5-isopropyl-3,6-dimethoxy-2-methyl-2,5-dihydropyrazin-2-yl)-N,N-dimethylethanamine (5a): To a 75 mL high pressure reaction vessel were placed 4a (4.000 g, 13.11 mmol), DMAP (0.4803 g, 3.932 mmol), TEA (9.133 mL, 65.53 mmol), and dimethylamine in THF (13.11 mL, 26.21 mmol). The vessel was sealed and heated to 80° C. The mixture was cooled to room temperature. Additional dimethylamine (13.11 mL, 26.21 mmol) and TEA (d. 0.726) (9.133 mL, 65.53 mmol) were added to the vessel. The reaction mixture was heated to 80° C. and then cooled at room temperature. The reaction mixture was transferred to a flask and concentrated under reduced pressure. The residue was diluted with EtOAc (50 mL) and filtered through a medium frit filter. The brown solid was washed with EtOAc (2×20 mL). The filtrate was concentrated under reduced pressure to provide tan solid. The solid was purified by chromatography with 1:19 TEA/EtOAc to provide 2.166 g of 5a as a tan oil (61.4% yield). ¹H NMR (400 MHz, CDCl₃) δ 3.94 (d, J=3.51 Hz, 1H), 3.67 (s, 3H), 3.66 (s, 3H), 2.30-2.21 (m, 1H), 2.16 (s, 6H), 2.09-1.91 (m, 3H), 1.74-1.67 (m, 1H), 1.34 (s, 3H), 1.008 (d, J=7.03 Hz, 3H), 0.68 (d, J=7.03 Hz, 3H).

Step F: Preparation of 6a: To a round-bottomed flask were placed 5a (2.166 g, 8.041 mmol) and THF (10 mL). 1N HCl (50 mL) was added and the reaction mixture was stirred at room temperature for 2 days. The reaction mixture was then extracted with ether (2×20 mL), and the aqueous layer was saturated with NaCl (solid) and basified with concentrated NH₄OH solution. The mixture was transferred to a continuous extractor and extracted with ether continuously for 3 hours. The organic layer was dried over MgSO₄, filtered through a Celite pad, and concentrated under reduced pressure to provide 1.634 g of a crude yellow gel. The crude contained 4.404 mmol of compound 6a (54%; a mixture of (S)-methyl 2-amino-4-(dimethylamino)-2-methylbutanoate and 6.606 mmol of the side-product (R)-methyl 2-amino-3-methylbutanoate), which was used in the next step without purification.

Step G: Preparation of (S)-Methyl 2-(5-(2,4-difluorophenoxy)-1-isobutyl-1H-indazole-6-carboxamido)-4-(dimethylamino)-2-methylbutanoate (9a): To a round-bottomed flask were placed crude 6a from Step F, compound 8 prepared as described in Example 4 (5.370 g, 12.11 mmol), TEA (4.604 mL, 33.03 mmol) and CH₂Cl₂ (25 mL). The mixture was stirred at room temperature for 3 days, and then concentrated under reduced pressure. The crude residue was purified by chromatography with 100% EtOAc-5% TEA in EtOAc. Two fractions were collected: Fraction #1 (the valine methyl ester side-product) and Fraction #2: 0.946 g (mixed: 0.977 g). ¹H NMR of Fraction #2 and the mixed fractions were almost identical. Fraction #2 and the mixed fractions were dissolved in ether, filtered through a cotton plug, and concentrated under reduced pressure to provide 1.773 g of compound 9a as a viscous oil (80.1% yield; 43% yield for two steps). ¹H NMR (400 MHz, CDCl₃) δ 9.09 (br s, 1H), 8.27 (s, 1H), 7.86 (s, 1H), 7.11 (dt, J=5.47, 8.98 Hz, 1H), 7.04-6.99 (m, 1H), 7.03 (s, 1H), 6.93-6.87 (m, 1H), 4.21 (d, J=7.42 Hz, 2H), 3.72 (s, 3H), 2.41-2.26 (m, 3H), 2.26-2.19 (m, 1H), 2.12-2.05 (m, 1H), 2.07 (s, 6H), 1.70 (s, 3H), 0.92 (d, J=6.64 Hz, 6H). MS ESI (+) m/z 503 (M+1) detected.

### Example 2

### (S)-methyl 2-(cyclopropylmethyl)-2-(5-(2,4-difluorophenoxy)-1-isobutyl-1H-indazole-6-carboxamido)-4-(dimethylamino)butanoate (9b)

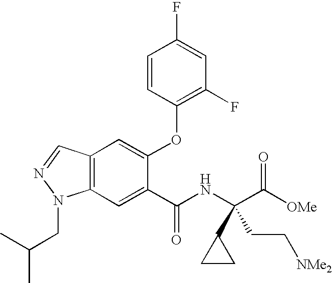

Step A: Preparation of (R)-2-isopropyl-3,6-dimethoxy-2,5-dihydropyrazine (1): Prepared as described in Example 1, Step A.

Step B: Preparation of 2-((2S,5R)-2-(cyclopropylmethyl)-5-isopropyl-3,6-dimethoxy-2,5-dihydropyrazin-2-yl)ethanol (2b): To a flame-dried 50 mL round-bottomed flask was placed 1 (1.001 g, 5.433 mmol) in THF (10 mL). The mixture was cooled to −78° C. and stirred for 0.5 hours. n-Butyl lithium (2.50 M in hexanes, 2.391 mL, 5.977 mmol) was added and the mixture was stirred at −78° C. for 1.5 hours. (Bromomethyl)cyclopropane (1.054 mL, 10.87 mmol) was added at −78° C. The mixture was stirred for 1 hour at −78° C. and placed in a freezer (−18° C.) for 18 hours. The mixture was warmed to room temperature and quenched with saturated NaHCO₃ solution (10 mL). The layers were separated and the aqueous layer was extracted with CH₂Cl₂ (3×5 mL). The combined organic layers were washed with brine (2×5 mL), dried over MgSO₄, filtered through a Celite pad, and concentrated under reduced pressure to provide 1.648 g of oil. The crude oil was purified by chromatography with 1:9 ether/hexanes to provide two fractions: mixed (Fraction #1 and Fraction #2; 1.079 g) and Fraction #2 (0.177 g), which provided 1.256 g of 2b as an oil (97.0% yield). Fraction #1: ¹H NMR (400 MHz, CDCl₃) δ 4.13-4.07 (m, 1H), 3.98-3.95 (m, 1H), 3.70 (s, 3H), 3.69 (s, 3H), 2.35-2.25 (m, 1H), 1.77-1.63 (m, 2H), 1.33-1.24 (m, 1H), 1.07 (d, J=7.03 Hz, 3H), 0.70 (d, J=7.03 Hz, 3H), 0.42-0.32 (m, 2H), 0.09-0.02 (m, 2H). Fraction #2: ¹H NMR (400 MHz, CDCl₃) δ 4.09 (dt, J=7.42, J=4.69 Hz, 1H), 3.95-3.92 (m, 1H), 3.69 (s, 3H), 3.67 (s, 3H), 2.28-2.16 (m, 1H), 1.71-1.56 (m, 2H), 1.07 (d, J=6.64 Hz, 3H), 0.97-0.86 (m, 1H), 0.75 (d, J=6.64 Hz, 3H), 0.49-0.40 (m, 2H), 0.13-0.08 (m, 2H).

Step C: Preparation of 2-((2S 5R)-2-(cyclopropylmethyl)-5-isopropyl-3,6-dimethoxy-2,5-dihydropyrazin-2-yl)ethanol (3b): To a flask was added 2b (1.256 g, 5.270 mmol) in THF (10 mL), and the reaction mixture was cooled to −78° C. After 0.5 hours at −78° C., n-butyl lithium (2.50 M in hexanes, 4.848 mL, 12.12 mmol) was added. After 1.5 hours at −78° C., 2-bromoethanol (0.4109 mL, 5.797 mmol) was added. The mixture was stirred at −78° C. for 2 hours and slowly warmed to −50° C. The bath temperature was maintained at −50±5° C. for 1 hour and then the mixture was placed in a freezer (−18° C.) for 2.5 days. The mixture was warmed to 0° C. and quenched with saturated NaHCO₃ solution (10 mL). The layers were separated and the aqueous layer was extracted with CH₂Cl₂ (3×50 mL). The combined organic layers were washed with brine (50 mL), dried over MgSO₄, filtered through a Celite pad, and concentrated under reduced pressure to provide 1.5 g of a colorless oil. The crude oil was purified by chromatography with 1:1 ether/hexanes to provide 1.020 g of 3b as a clear oil (68.5% yield). ¹H NMR (400 MHz, CDCl₃) δ 3.95 (d, J=3.51 Hz, 1H), 3.84-3.76 (m, 1H), 3.68 (s, 3H), 3.66 (s, 3H), 3.62-3.55 (m, 1H), 3.08-3.02 (m, 1H), 2.39-2.30 (m, 1H), 2.03-1.89 (m, 2H), 1.80-1.74 (m, 1H), 1.71-1.66 (m, 1H), 1.11 (d, J=6.64 Hz, 3H), 0.72 (d, J=6.64 Hz, 3H), 0.56-0.46 (m, 1H), 0.41-0.26 (m, 2H), 0.10 (0.01 (m, 2H).

Step D: Preparation of (2S,5R)-2-(2-bromoethyl)-2-(cyclopropylmethyl)-5-isopropyl-3,6-dimethoxy-2,5-dihydropyrazine (4b): To a round-bottomed flask were added 3b (1.020 g, 3.612 mmol), CBr₄ (1.437 g, 4.335 mmol) and CH₂Cl₂ (10 mL). The mixture was cooled in an ice bath and stirred for 15 minutes. PPh₃ (1.421 g, 5.418 mmol) was added portionwise at 0° C. The mixture was warmed to room temperature after 40 minutes in an ice bath, and then the reaction mixture was concentrated under reduced pressure. Ether (10 mL) was added to the residue, and white solid crashed out. The slurry was stirred vigorously for 0.5 hours and then filtered through a medium frit funnel. The filtrate was concentrated under reduced pressure and purified by chromatography with 1:19 ether/hexanes to provide 1.028 g of 4b as an oil (82.4% yield) after drying overnight under high vacuum. ¹H NMR (400 MHz, CDCl₃) δ 3.91 (d, J=3.90 Hz, 1H), 3.69 (s, 3H), 3.67 (s, 3H), 3.18-3.05 (m, 2H), 2.38-2.27 (m, 2H), 2.21-2.13 (m, 1H), 1.64 (d, J=7.03 Hz, 2H), 1.10 (d, J=7.03 Hz, 3H), 0.70 (d, J=7.03 Hz, 3H), 0.60-0.49 (m, 1H), 0.41-0.34 (m, 1H), 0.32-0.25 (m, 1H), 0.09-0.01 (m, 2H).

Step E: Preparation of 2-((2S,5R)-2-(cyclopropylmethyl)-5-isopropyl-3,6-dimethoxy-2,5-dihydropyrazin-2-yl)-N,N-dimethylethanamine (5b): To a 35 mL high pressure reaction vessel were placed 4b (0.956 g, 2.769 mmol), dimethylamine in THF (2.769 mL, 5.538 mmol), DMAP (0.1015 g, 0.8306 mmol), and TEA (1.930 mL, 13.84 mmol). The vessel was sealed and heated to 80° C. for 16 hours. The mixture was cooled to room temperature. Additional dimethylamine (2.769 mL, 5.538 mmol) and TEA (1.930 mL, 13.84 mmol) were added and the vessel was sealed. The mixture was heated to 80° C. for additional 4 days. The mixture was cooled to room temperature, and the reaction mixture was transferred to a flask and concentrated under reduced pressure. The residue was purified by chromatography with 1:19 TEA/EtOAc to provide 0.649 g of 5b as a brown oil (75.8% yield). ¹H NMR (400 MHz, CDCl₃) δ 3.90 (d, J=3.51 Hz, 1H), 3.69 (s, 3H), 3.66 (s, 3H), 2.39-2.29 (m, 1H), 2.15 (s, 6H), 2.03-S 1.96 (m, 2H), 1.94-1.86 (m, 1H), 1.80-1.71 (m, 1H), 1.65 (d, J=7.03 Hz, 2H), 1.11 (d, J=7.03 Hz, 3H), 0.70 (d, J=6.64 Hz, 3H), 0.60-0.49 (m, 1H), 0.39-0.32 (m, 1H), 0.31-0.24 (m, 1H), 0.09-0.01 (m, 2H).

Step F: Preparation of 6b: To a round-bottomed flask was added 5b (0.647 g, 2.09 mmol) in THF (5 mL). 1N HCl (20 mL) was added and the mixture was stirred at room temperature for 6 days. The reaction mixture was extracted with ether (2×10 mL) and the aqueous layer was basified with concentrated NH₄OH (5 mL). The aqueous layer was saturated with solid NaCl and extracted with ether (4×10 mL). The combined extracts were dried over MgSO₄, filtered through a Celite pad, and concentrated under reduced pressure to provide 583 mg of oil containing crude 6b (mass balance 81%). The crude reaction mixture was used in the next step without further purification.

Step G: Preparation of (S)-methyl 2-(cyclopropylmethyl)-2-(5-(2,4-difluorophenoxy)-1-isobutyl-1H-indazole-6-carboxamido)-4-(dimethylamino)butanoate (9b): To a round-bottomed flask were added the crude 6b (0.583 g, 1.688 mmol), compound 8 (0.8979 g, 2.025 mmol; prepared as described in Example 4), TEA (0.7056 mL, 5.063 mmol), and CH₂Cl₂ (15 mL). The mixture was stirred for 2 days at room temperature and then concentrated under reduced pressure. The residue was purified by chromatography with EtOAc-5% TEA in EtOAc to provide 390 mg of viscous oil. The oil was purified again with 5% TEA in ether to provide 138 mg of compound 9b (15%). ¹H NMR (400 MHz, CDCl₃) δ 8.97 (br s, 1H), 8.30 (s, 1H), 7.86 (s, 1H), 7.16 (dt, J=5.47, 8.98 Hz, 1H), 7.04-6.98 (m, 1H), 7.03 (s, 1H), 6.95-6.88 (m, 1H), 4.21 (d, J=7.42 Hz, 2H), 3.71 (s, 3H), 2.78-2.67 (m, 2H), 2.42-2.31 (m, 1H), 2.30-2.19 (m, 1H), 2.17-2.04 (m, 2H), 2.07 (s, 6H), 1.70-1.64 (m, 1H), 0.94 (d, J=6.64 Hz, 6H), 0.61-0.50 (m, 1H), 0.37-0.23 (m, 2H), 0.13-0.04 (m, 1H), −0.01 to −0.08 (m, 1H).

### Example 3

### (S)-Methyl 2-(5-(2,4-difluorophenoxy)-1-isobutyl-1H-indazole-6-carboxamido)-2-methyl-4-(pyrrolidin-1-yl)butanoate (9c)

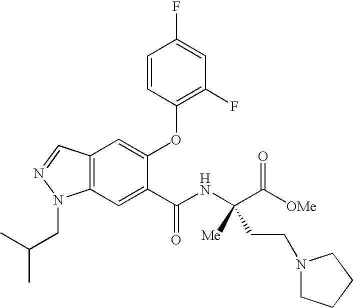

Steps A-D: Preparation of (2S,5R)-2-(2-bromoethyl)-5-isopropyl-3,6-dimethoxy-2-methyl-2,5-dihydropyrazine, 4a: Compound 4a was prepared as described in Example 1, Steps A-D.

Step E: Preparation of (2S,5R)-5-isopropyl-3,6-dimethoxy-2-methyl-2-(2-(pyrrolidin-1-yl)ethyl)-2,5-dihydropyrazine (5c): To a 15 mL high pressure reaction vessel were added 4a (0.500 g, 1.638 mmol), DMAP (0.06004 g, 0.4915 mmol), pyrrolidine (0.2735 mL, 3.276 mmol), TEA (1.142 mL, 8.191 mmol) and THF (5 mL). The vessel was sealed and heated to 80° C. for 16 hours and then cooled to room temperature and concentrated under reduced pressure. The residue was purified by chromatography with 1:19 TEA/EtOAc to provide 0.355 g of 5c as a pale yellow oil (73.4% yield). ¹H NMR (400 MHz, CDCl₃) δ 3.94 (d, J=3.12 Hz, 1H), 3.66 (s, 3H), 3.65 (s, 3H), 2.46-2.39 (m, 4H), 2.29-2.18 (m, 2H), 2.15-2.00 (m, 2H), 1.82-1.71 (m, 5H), 1.34 (s, 3H), 1.07 (d, J=7.03 Hz, 3H), 0.68 (d, J=7.03 Hz, 3H).

Step F: Preparation of compound 6c: To a round-bottomed flask were placed 5c (0.353 g, 1.19 mmol) and THF (2 mL). 1N HCl (10 mL) was added the mixture was stirred at room temperature. After 2 days, the mixture was extracted with ether (2×10 mL) and the aqueous layer was basified with concentrated NH₄OH (5 mL). The aqueous layer was saturated with solid NaCl and extracted with ether (3×10 mL). The combined extracts were dried over MgSO₄, filtered through a Celite pad, and concentrated under reduced pressure to provide 309 mg of oil (mass balance: 78%). ¹H NMR (crude) showed 1:1 ratio of compound 6c and the valine methyl ester. The crude reaction mixture was used in the next step without purification.

Step G: Preparation of (S)-Methyl 2-(5-(2,4-difluorophenoxy)-1-isobutyl-1H-indazole-6-carboxamido)-2-methyl-4-(pyrrolidin-1-yl)butanoate (9c): To a round-bottomed flask were placed crude 6c (0.309 g, 0.9323 mmol), compound 8 (0.4547 g, 1.025 mmol), TEA (0.3898 mL, 2.797 mmol; prepared as described in Example 4) and CH₂Cl₂ (10 mL). The mixture was stirred at room temperature for 3 days. The reaction mixture was concentrated under reduced pressure, and the crude residue was purified by chromatography eluting with 5% TEA in EtOAc. Fractions 15-25 were combined and chromatographed again using 5% TEA in EtOAc to provide 98 mg of 9c as an oil (19.9% yield; 15.5% yield for two steps). ¹H NMR (400 MHz, CDCl₃) δ 9.02 (br s, 1H), 8.22 (s, 1H), 7.88 (s, 1H), 7.10 (s, 1H), 7.07-6.98 (m, 2H), 6.90-6.84 (m, 1H), 4.21 (d, J=7.42 Hz, 2H), 3.70 (s, 3H), 2.53-2.48 (m, 1H), 2.46-2.31 (m, 1H), 2.14-2.08 (m, 1H), 1.68 (s, 3H), 1.67-1.61 (m, 4H), 0.93 (d, J=7.03 Hz, 6H). MS ESI (+) m/z 529 (M+1) detected.

### Example 4

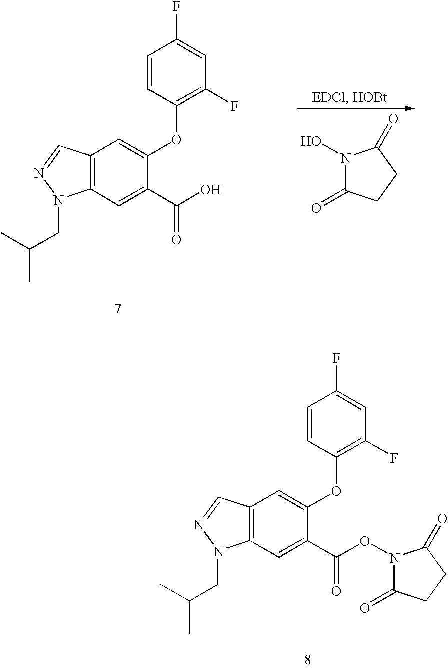

### 5-(2,4-difluorophenoxy)-1-isobutyl-1H-indazole-6-carboxylic acid 2,5-dioxo-pyrrolidin-1-yl ester (8)

This example describes the synthesis of compound (8), which can be used to prepare compounds of this invention.

Step A: 1,2-Dibromo-4-methyl-5-nitrobenzene: 3,4-dibromotoluene (108.11 mL, 800 mmol) was added dropwise with mechanical stirring over 4 hours to nitric acid (90%, 280 mL, 6000 mmol) that was cooled to 0° C. under a nitrogen atmosphere. The internal temperature of the mixture was maintained below 10° C. during the addition and the reaction mixture was stirred for 1 hour at 0° C. after completion of addition. Water (840 mL) was added drop-wise to the mixture while maintaining the internal temperature below 10° C. The crude product was collected by filtration and washed with water (5×500 mL) to remove the excess nitric acid. The solids were dried under high vacuum and purified by recrystallization from ethanol (800 mL) to provide 180.9 g (77% yield) of the desired product as a solid. ¹H NMR (400 MHz, CDCl₃) δ 8.24 (s, 1H), 7.64 (s, 1H), 2.55 (s, 3H).

Step B: 1-Bromo-2-(2,4-difluorophenoxy)-4-methyl-5-nitrobenzene: A mixture of 1,2-dibromo-4-methyl-5-nitrobenzene (84.3 g, 286 mmol), 2,4-difluorophenol (37.2 g, 286 mmol), and K₂CO₃ (43.5 g, 315 mmol) were heated to 100° C. for 45 hours. The reaction mixture was cooled to room temperature and then stored in a 5° C. refrigerator overnight. The reaction mixture was poured into 1200 mL of ice water. The resulting damp solid was collected, partially ground up, and stirred in 900 mL H₂O for 45 minutes. The solid was collected by filtration and rinsed with 700 mL of water portion-wise. The resulting solid was dried under high vacuum overnight to yield 93.5 g of the desired product as a brown solid (95% yield). ¹H NMR (400 MHz, CDCl₃) δ 8.38 (s, 1H), 7.18 (m, 1H), 7.03 (m, 1H), 6.97 (m, 1H), 6.52 (s, 1H), 2.50 (s, 3H).

Step C: 5-Bromo-4-(2,4-difluorophenoxy)-2-methylphenylamine: 1-Bromo-2-(2,4-difluorophenoxy)-4-methyl-5-nitrobenzene (87.0 g, 253 mmol) was dissolved in THF (300 mL) and diluted with MeOH (900 mL). Zinc dust (82.7 g, 1.26 mol) was added and 1 L of saturated NH₄Cl was added slowly so that the reaction temperature never exceeded 42° C. The reaction mixture was mechanically stirred vigorously for 16 hours, and the filtered through Celite and the filter cake was washed with ethyl acetate. The filtrate was then concentrated with 1.2 L of saturated NH₄OAc. The THF/MeOH was removed and the solids were collected and washed with water. The solids were then stirred in 1 L water for 30 minutes, then collected via filtration and rinsed with water (1 L) in three portions. The resulting solid was dried under high vacuum for 48 hours to produce 64 g of the desired product (81% yield). MS (ESI+) m/z 314,316 (M+1, Br pattern) detected; ¹H NMR (400 MHz, CDCl₃) δ 6.92 (m, 1H), 6.91 (s, 1H), 6.75 (m, 2H), 6.70 (s, 1H), 3.57 (br. s, 2H), 2.08 (s, 3H).

Step D: 6-Bromo-5-(2,4-difluorophenoxy)-1H-indazole:

(i) 5-bromo-4-(2,4-difluorophenoxy)-2-methylbenzenediazonium tetrafluoroborate: 5-Bromo-4-(2,4-difluorophenoxy)-2-methylphenylamine (30.0 g, 96 mmol) was dissolved in 2:1 AcOH/H₂O (960 mL). NH₄BF₄ (20.0 g, 191 mmol) was added and the reaction mixture was cooled to 3° C. (−30 minutes). Concentrated HCl (40 mL) was then added, during which the mixture warmned to 6° C. The mixture was cooled to 2° C. and then NaNO₂ (7.25 g, 105 mmol) was added. The reaction mixture was stirred in an ice bath for 5 minutes and then allowed to stir for 1 hour at room temperature. The mixture was concentrated under reduced pressure and the residue was azeotroped with toluene (3×400 mL). The crude material was used in the next reaction without further purification.

(ii) 6-Bromo-5-(2,4-difluorophenoxy)-1H-indazole: The crude 5-bromo-4-(2,4-difluorophenoxy)-2-methylbenzenediazonium tetrafluoroborate was suspended in ethyl acetate (650 mL) and treated with 10 equivalents of KOAc. The mixture was vigorously stirred at room temperature for 1.5 hours and then filtered and diluted to a 1 L total volume with ethyl acetate. The mixture was washed with saturated NaHCO₃/brine (800 mL, 1:1). The aqueous phase was extracted with ethyl acetate (400 mL). The organics were combined, dried (MgSO₄) and concentrated to provide the desired product as a brown solid (31 g, 99% yield). ¹H NMR (400 MHz, CDCl₃) δ 10.55 (br. s, 1H), 7.98 (s, 1H), 7.84 (s, 1H), 7.20 (s, 1H), 6.99 (m, 1H), 6.94 (m, 1H), 6.84 (m, 1H).

Step E: 6-Bromo-5-(2,4-difluorophenoxy)-1-isobutyl-1H-indazole: 6-Bromo-5-(2,4-difluorophenoxy)-1H-indazole (60.0 g, 185 mmol) was dissolved in DMF and treated with K₂CO₃ (76.5 g, 554 mmol) and isobutyl bromide (126.4 g, 923 mmol). The reaction mixture was stirred and heated to 80° C. for 16 hours. An additional 15 g of K₂CO₃ were added and the mixture was vigorously stirred for an additional 24 hours, then cooled to room temperature and filtered. The filtrate was concentrated under reduced pressure and dissolved in ether (1 L). The ether layer was washed with 1:5 brine/water (2×600 mL). The aqueous phases were extracted with ether (300 mL) and the combined organic layers were dried (MgSO₄) and concentrated under reduced pressure. The crude product was chromatographed on a Biotage Flash 75 in two batches (about 35 g each) eluting with 5% ethyl acetate in hexanes. The combined purified products yielded 30.1 g of the desired product as a solid (43% yield). MS (ESI+) m/z 381, 383 (M+1, Br pattern) detected; ¹H NMR (400 MHz, CDCl₃) δ 7.86 (s, 1H), 7.72 (s, 1H), 7.16 (s, 1H), 6.98 (m, 1H), 6.92 (m, 1H), 6.82 (m, 1H), 4.12 (d, 2H), 2.34 (m, 1H), 0.94 (d, 6H).

Step F: 5-(2,4-Difluorophenoxy)-1-isobutyl-1H-indazole-6-carbonitrile: 6-Bromo-5-(2,4-difluorophenoxy)-1-isobutyl-1H-indazole (31.2 g, 82 mmol) and Cu(I)CN (13.9 g, 156 mmol) were dissolved in DMA and degassed with nitrogen under vacuum. The reaction mixture was heated to 150° C. for 16 hours. The mixture was then cooled to room temperature and diluted with ethyl acetate before washing twice with 7M NH₄OH. The organic layer was washed with brine and degassed with nitrogen before being dried over MgSO₄ and concentrated under reduced pressure. The crude product was chromatographed eluting with 10% ethyl acetate in hexanes to afford 25.1 g of the desired product (95% yield).

Step G: 5-(2,4-Difluorophenoxy)-1-isobutyl-1H-indazole-6-carboxylic acid (7): 5-(2,4-Difluorophenoxy)-1-isobutyl-1H-indazole-6-carbonitrile (25.1 g, 77 mmol) was suspended in ethanol (620 mL) and KOH (2.5 M, 310 mL) and heated to reflux for 24 hours. The reaction mixture was cooled to room temperature and the ethanol was removed under reduced pressure. The resulting aqueous solution was diluted with water and washed with ether. The aqueous layer was acidified with concentrated HCl to pH 1 and extracted with ethyl acetate several times. The organic layers were combined and concentrated under reduced pressure to afford 25.5 g of the desired product (96% yield). ¹H NMR (400 MHz, CDCl₃) δ 8.37 (s, 1H), 7.91 (s, 1H), 7.20 (m, 1H), 7.07 (s, 1H), 7.04 (m, 1H), 6.95 (m, 1H), 4.24 (d, 2H), 2.36 (m, 1H), 0.94 (d, 6H).

Step H: 5-(2,4-difluorophenoxy)-1-isobutyl-1H-indazole-6-carboxylic acid 2,5-dioxopyrrolidin-1-yl ester (8): The acid 7 (39.5 g, 113.9 mmol), N-hydroxysuccinimide (17.0 g, 148 mmol) and EDCI (26.0 g, 137 mmol) were dissolved in CH₂Cl₂ (200 mL). The solution was stirred for 3 hours and then was diluted with 100 mL CH₂Cl₂ and washed sequentially with a saturated NH₄Cl solution, twice with a saturated Na₂CO₃ solution, and once with brine. The organics were dried (MgSO₄), filtered, and concentrated under reduced pressure. The crude residue was precipitated from Et₂O to afford 41.0 g of compound 8 (81% yield). ¹H NMR (400 MHz, CDCl₃) δ 8.26 (s, 1H), 7.95 (s, 1H), 7.20 (s, 1H), 7.05-6.94 (m, 2H), 6.88-6.81 (m, 1H), 4.24 (d, J=7.83 Hz, 2H), 2.90 (s, 4H), 2.43-2.32 (m, 1H), 0.96 (d, J=7.04 Hz, 6H).

Compounds 11a and 11c were prepared as shown in FIG. 2 and as described in Examples 5-6.

### Example 5

### (S)-N-(1-amino-4-(dimethylamino)-2-methyl-1-oxobutan-2-yl)-5-(2,4-difluorophenoxy)-1-isobutyl-1H-indazole-6-carboxamide (11a)

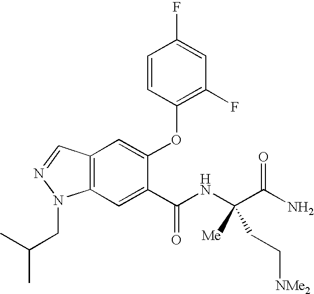

Step A: To a round-bottomed flask were added 9a (1.773 g, 3.528 mmol), TMSOK (1.257 g, 8.820 mmol) and THF (50 mL). The mixture was stirred at room temperature for 1 day. HCl in dioxane (8.820 mL, 35.28 mmol) was added and the reaction mixture was stirred for 1 hour at room temperature. The reaction mixture was then concentrated under reduced pressure and dried under high vacuum for 4 hours to provide 2.835 g of compound 10a as a foamy solid. The crude product was used in the next step without purification.

Step B: To a round-bottomed flask were added 10a (3.528 mmol), EDCI (0.8792 g, 4.586 mmol), HOBT-H₂O (0.7024 g, 4.586 mmol) and CH₂Cl₂ (25 mL). TEA (2.459 mL, 17.64 mmol) was added. The mixture was stirred for 1.5 hour at room temperature, after which NH₃ (0.5 M in dioxane; 70.56 mL, 35.28 mmol) was added. A white solid fell out of solution. The mixture was stirred at room temperature for 3 hours, then filtered through a Celite pad. The filtrate was concentrated under reduced pressure to provide 3.34 g of pale yellow oil. The crude oil was purified by chromatography eluting with 1:1:8 MeOH/TEA/EtOAc to provide 1.375 g of 11a as a solid (79.9% for two steps). ¹H NMR (400 MHz, CDCl₃) δ 9.52 (br s, 1H), 8.11 (s, 1H), 7.88 (s, 1H), 7.66 (br s, 1H), 7.12-7.05 (m, 2H), 7.07 (s, 1H), 7.04-6.97 (m, 1H), 6.92-6.85 (m, 1H), 5.26 (br s, 1H), 4.20 (d, J=7.42 Hz, 2H), 2.60-2.53 (m, 1H), 2.43-2.33 (m, 2H), 2.27-2.19 (m, 1H), 2.14 (s, 6H), 2.14-2.08 (m, 1H), 1.73 (s, 3H), 0.93 (d, J=7.03 Hz, 6H). MS ESI (+) m/z 488 (M+1) detected.

### Example 6

### (S)-N-(1-amino-2-methyl-1-oxo-4-(pyrrolidin-1-yl)butan-2-yl)-5-(2,4-difluorophenoxy)-1-isobutyl-1H-indazole-6-carboxamide (11c)

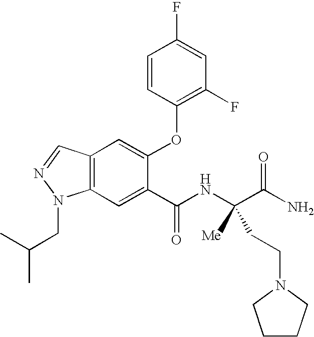

Step A: To a round-bottomed flask were added 9c (0.098 g, 0.1854 mmol), TMSOK (0.06607 g, 0.4635 mmol), and THF (2 mL). The mixture was stirred at room temperature for 1 day. HCl (4.0 M in dioxane; 0.4635 mL, 1.854 mmol) was added to the mixture and the mixture was stirred for 1 hour at room temperature. The mixture was concentrated under reduced pressure and dried under high vacuum for 4 hours to provide 154 mg of 10c as a foamy solid. The crude solid was used for the next step without purification.

Step B: To a round-bottomed flask were placed 10c (0.1854 mmol), EDCI (0.04620 g, 0.2410 mmol), HOBT-H₂O (0.03691 g, 0.2410 mmol), and CH₂Cl₂ (5 mL). TEA (0.1292 mL, 0.9270 mmol) was added. The mixture was stirred at room temperature for 1.5 hours, after which NH₃ (0.5M in dioxane; 3.708 mL, 1.854 mmol) was added, and a white solid fell out of solution. The mixture was stirred for 3 hours at room temperature, and then filtered through a Celite pad. The filtrate was concentrated under reduced pressure to provide 150 mg of colorless oil. The crude oil was purified by chromatography eluting with 1:9 TEA/EtOAc containing 5% MeOH to provide 62 mg of 11c as a solid. ¹H NMR (400 MHz, CDCl₃) δ 9.86 (br s, 1H), 7.98 (s, 1H), 7.90 (s, 1H), 7.73 (br s, 1H), 7.14 (s, 1H), 7.03-6.96 (m, 2H), 6.88-6.82 (m, 1H), 5.23 (br s, 1H), 4.20 (d, J=7.42 Hz, 2H), 2.85-2.78 (m, 1H), 2.60-2.50 (m, 3H), 2.49-2.41 (m, 2H), 2.41-2.33 (m, 1H), 2.33-2.22 (m, 1H), 2.17-2.08 (m, 1H), 1.72 (s, 3H), 1.63-1.53 (m, 4H), 0.93 (d, J=6.64 Hz, 6H). MS ESI (+) m/z 514 (M+1) detected.

Compound 12a was prepared as shown in FIG. 2 and as described in Example 7.

### Example 7

### (S)-5-(2,4-difluorophenoxy)-N-(4-(dimethylamino)-1-hydroxy-2-methylbutan-2-yl)-1-isobutyl-1H-indazole-6-carboxamide (12a)

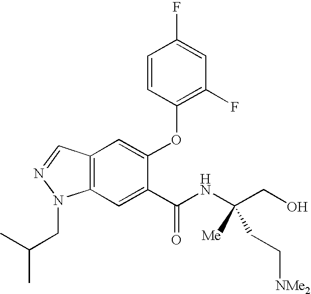

To a round-bottomed flask were added 9a (115 mg, 229 μmol) and 5 mL of THF-MeOH (4:1). NaBH₄ (43 mg, 1144 μmol) was added and the mixture was heated under reflux for 5 hours. The reaction mixture was concentrated under reduced pressure and the residue was treated with CH₂Cl₂. The resulting slurry was purified by chromatography with 1:9 MeOH/CH₂Cl₂ to provide 63 mg (58%) of compound 12a, which became foamy solid upon ether treatment. ¹H NMR (400 MHz, CDCl₃) δ 8.39 (br s, 1H), 8.24 (s, 1H), 7.86 (s, 1H), 7.13 (dt, J=5.47, 8.98 Hz, 1H), 7.03-6.96 (m, 1H), 7.02 (s, 1H), 6.93-6.86 (m, 1H), 4.21 (d, J=7.42 Hz, 2H), 3.42 (s, 2H), 2.68-2.58 (m, 2H), 2.41-2.31 (m, 1H), 2.23-2.11 (m, 1H), 2.19 (s, 6H), 1.78-1.67 (m, 1H), 1.46 (s, 3H), 0.93 (d, J=8.59 Hz, 6H). MS ESI (+) m/z 475 (M+1) detected.

Additional compounds of the present invention include compounds of general Formulas Ia-Id as shown in Tables 1-4.

TABLE 1

| | |
|---|---|
| | Ia |
| 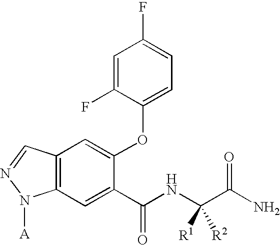 | |

| Example | | | |
|---|---|---|---|
| # | A | R¹ | R² |
| 8 | Me | Me | CH₂CH₂NH₂ |
| 9 | Me | Me | CH₂CH₂NHMe |
| 10 | Me | Me | CH₂CH₂NHEt |
| 11 | Me | Me | CH₂CH₂NHPr |
| 12 | Me | Me | CH₂CH₂NH(i-Pr) |
| 13 | Me | Me | CH₂CH₂NMe₂ |
| 14 | Me | Me | CH₂CH₂NEt₂ |
| 15 | Me | Me | CH₂CH₂N(n-Pr)₂ |
| 16 | Me | Me | 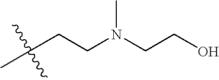 |
| 17 | Me | Me | 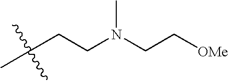 |
| 18 | Me | Me | 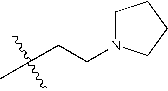 |
| 19 | Me | Me | 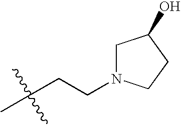 |
| 20 | Me | Me | 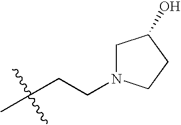 |
| 21 | Me | Me | 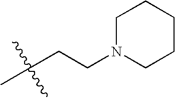 |
| 22 | Me | Me | 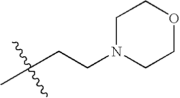 |
| 23 | Me | Et | CH₂CH₂NH₂ |
| 24 | Me | Et | CH₂CH₂NHMe |
| 25 | Me | Et | CH₂CH₂NHEt |
| 26 | Me | Et | CH₂CH₂NHPr |
| 27 | Me | Et | CH₂CH₂NH(i-Pr) |
| 28 | Me | Et | CH₂CH₂NMe₂ |
| 29 | Me | Et | CH₂CH₂NEt₂ |
| 30 | Me | Et | CH₂CH₂N(n-Pr)₂ |
| 31 | Me | Et | 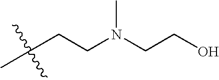 |
| 32 | Me | Et | 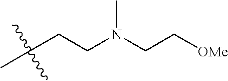 |
| 33 | Me | Et | 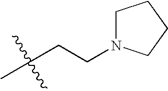 |
| 34 | Me | Et | 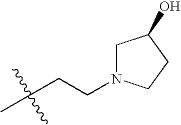 |
| 35 | Me | Et | 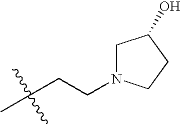 |
| 36 | Me | Et | 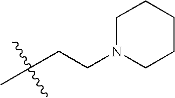 |
| 37 | Me | Et | 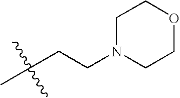 |
| 38 | Me | 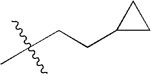 | CH₂CH₂NH₂ |
| 39 | Me | 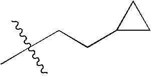 | CH₂CH₂NHMe |
| 40 | Me | 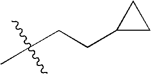 | CH₂CH₂NHEt |
| 41 | Me | 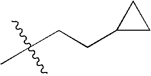 | CH₂CH₂NHPr |
| 42 | Me | 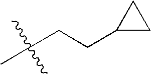 | CH₂CH₂NH(i-Pr) |
| 43 | Me | 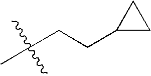 | CH₂CH₂NMe₂ |
| 44 | Me | 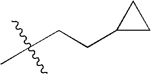 | CH₂CH₂NEt₂ |
| 45 | Me | 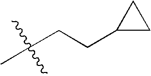 | CH₂CH₂N(n-Pr)₂ |
| 46 | Me | 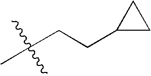 | 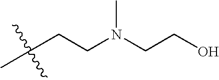 |
| 47 | Me | 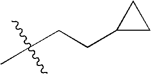 | 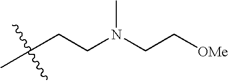 |
| 48 | Me | 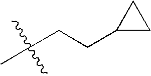 | 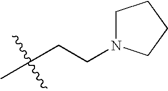 |
| 49 | Me | 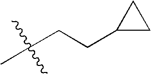 | 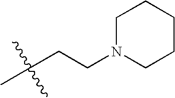 |
| 50 | Me | 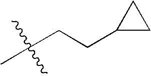 | 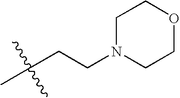 |
| 51 | 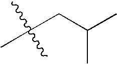 | Me | CH₂CH₂NH₂ |
| 52 | 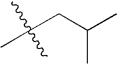 | Me | CH₂CH₂NHMe |
| 53 | 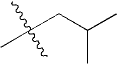 | Me | CH₂CH₂NHEt |
| 54 | 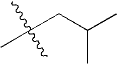 | Me | CH₂CH₂NHPr |
| 55 | 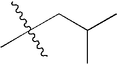 | Me | CH₂CH₂NH(i-Pr) |
| 56 | 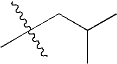 | Me | CH₂CH₂NEt₂ |
| 57 | 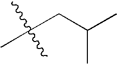 | Me | CH₂CH₂N(n-Pr)₂ |
| 58 | 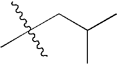 | Me | 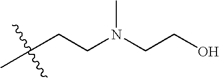 |
| 59 | 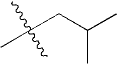 | Me | 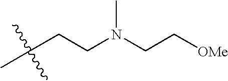 |
| 60 | 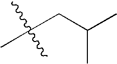 | Me | 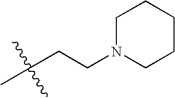 |
| 61 | 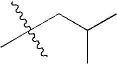 | Me | 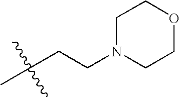 |
| 62 | 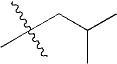 | Et | CH₂CH₂NH₂ |
| 63 | 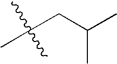 | Et | CH₂CH₂NHMe |
| 64 | 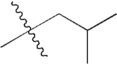 | Et | CH₂CH₂NHEt |
| 65 | 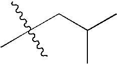 | Et | CH₂CH₂NHPr |
| 66 | 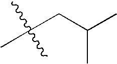 | Et | CH₂CH₂NH(i-Pr) |
| 67 | 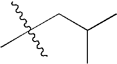 | Et | CH₂CH₂NMe₂ |
| 68 | 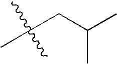 | Et | CH₂CH₂NEt₂ |
| 69 | 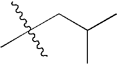 | Et | CH₂CH₂N(n-Pr)₂ |
| 70 | 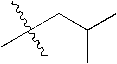 | Et | 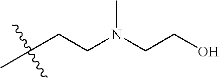 |
| 71 | 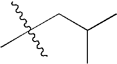 | Et | 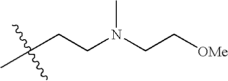 |
| 72 | 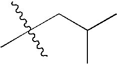 | Et | 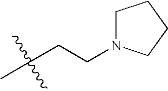 |
| 73 | 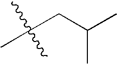 | Et | 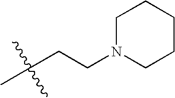 |
| 74 | 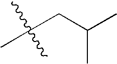 | Et | 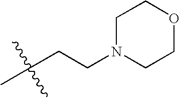 |
| 75 | 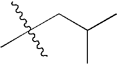 | 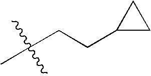 | CH₂CH₂NH₂ |
| 76 | 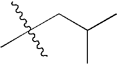 | 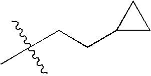 | CH₂CH₂NHMe |
| 77 | 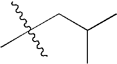 | 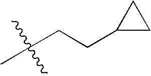 | CH₂CH₂NHEt |
| 78 | 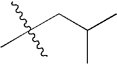 | 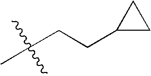 | CH₂CH₂NHPr |
| 79 | 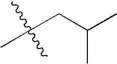 | 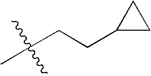 | CH₂CH₂NH(i-Pr) |
| 80 | 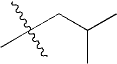 | 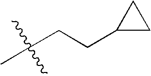 | CH₂CH₂NMe₂ |
| 81 | 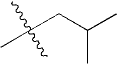 | 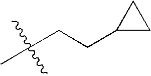 | CH₂CH₂NEt₂ |
| 82 | 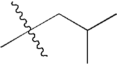 | 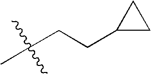 | CH₂CH₂N(n-Pr)₂ |
| 83 | 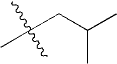 | 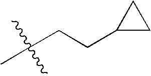 | 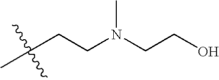 |
| 84 | 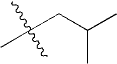 | 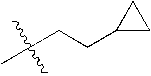 | 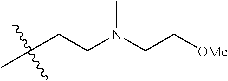 |
| 85 | 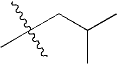 | 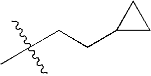 | 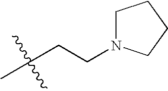 |
| 86 | 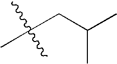 | 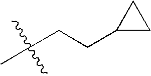 | 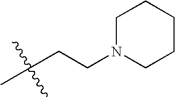 |
| 87 | 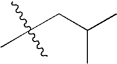 | 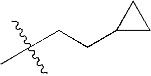 | 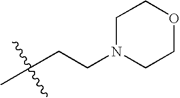 |
| 88 | 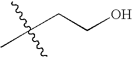 | Me | CH₂CH₂NH₂ |
| 89 | 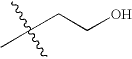 | Me | CH₂CH₂NHMe |
| 90 | 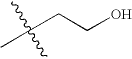 | Me | CH₂CH₂NHEt |
| 91 | 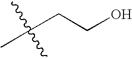 | Me | CH₂CH₂NHPr |
| 92 | 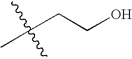 | Me | CH₂CH₂NH(i-Pr) |
| 93 | 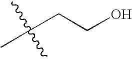 | Me | CH₂CH₂NMe₂ |
| 94 | 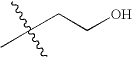 | Me | CH₂CH₂NEt₂ |
| 95 | 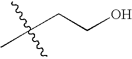 | Me | CH₂CH₂N(n-Pr)₂ |
| 96 | 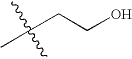 | Me | 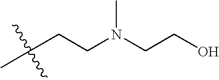 |
| 97 | 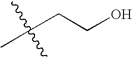 | Me | 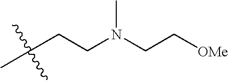 |
| 98 | 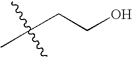 | Me | 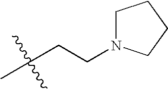 |
| 99 | 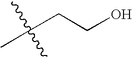 | Me | 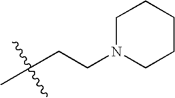 |
| 100 | 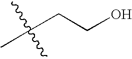 | Me | 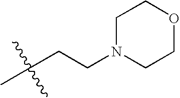 |
| 101 | 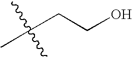 | Et | CH₂CH₂NH₂ |
| 102 | 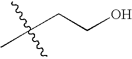 | Et | CH₂CH₂NHMe |
| 103 | 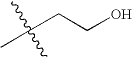 | Et | CH₂CH₂NHEt |
| 104 | 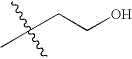 | Et | CH₂CH₂NHPr |
| 105 | 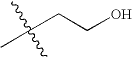 | Et | CH₂CH₂NH(i-Pr) |
| 106 | 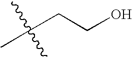 | Et | CH₂CH₂NMe₂ |
| 106 | 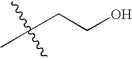 | Et | CH₂CH₂NEt₂ |
| 108 | 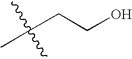 | Et | CH₂CH₂N(n-Pr)₂ |
| 109 | 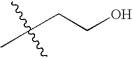 | Et | 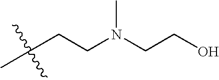 |
| 110 | 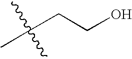 | Et | 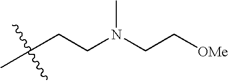 |
| 111 | 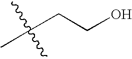 | Et | 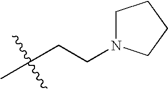 |
| 112 | 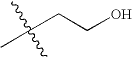 | Et | 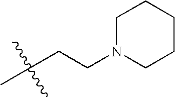 |
| 113 | 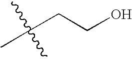 | Et | 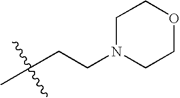 |
| 114 | 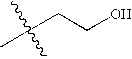 | 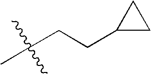 | CH₂CH₂NH₂ |
| 115 | 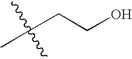 | 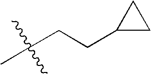 | CH₂CH₂NHMe |
| 116 | 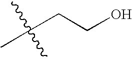 | 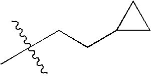 | CH₂CH₂NHEt |
| 117 | 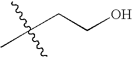 | 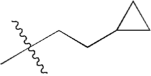 | CH₂CH₂NHPr |
| 118 | 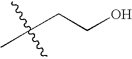 | 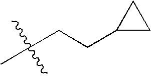 | CH₂CH₂NH(i-Pr) |
| 119 | 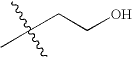 | 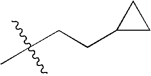 | CH₂CH₂NMe₂ |
| 120 | 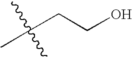 | 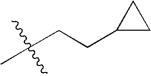 | CH₂CH₂NEt₂ |
| 121 | 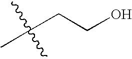 | 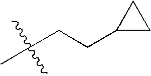 | CH₂CH₂N(n-Pr)₂ |
| 122 | 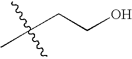 | 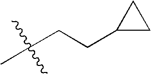 | 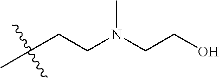 |
| 123 | 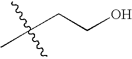 | 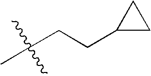 | 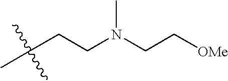 |
| 124 | 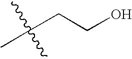 | 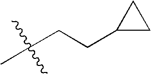 | 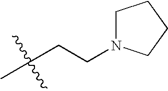 |
| 125 | 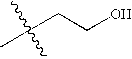 | 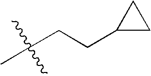 | 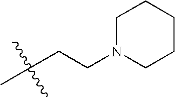 |
| 126 | 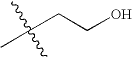 | 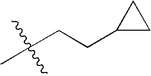 | 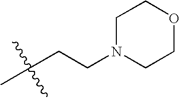 |
| 127 | 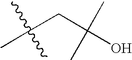 | Me | CH₂CH₂NH₂ |
| 128 | 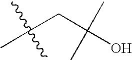 | Me | CH₂CH₂NHMe |
| 129 | 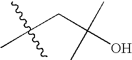 | Me | CH₂CH₂NHEt |
| 130 | 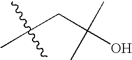 | Me | CH₂CH₂NHPr |
| 131 | 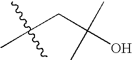 | Me | CH₂CH₂NH(i-Pr) |
| 132 | 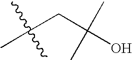 | Me | CH₂CH₂NMe₂ |
| 133 | 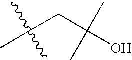 | Me | CH₂CH₂NEt₂ |
| 134 | 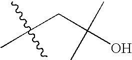 | Me | CH₂CH₂N(n-Pr)₂ |
| 135 | 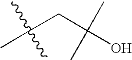 | Me | 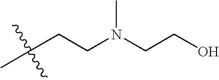 |
| 136 | 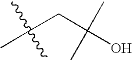 | Me | 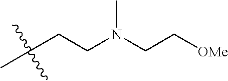 |
| 137 | 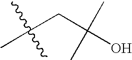 | Me | 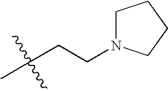 |
| 138 | 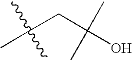 | Me | 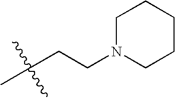 |
| 139 | 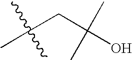 | Me | 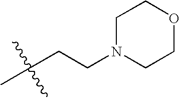 |
| 140 | 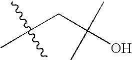 | Et | CH₂CH₂NH₂ |
| 141 | 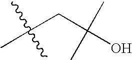 | Et | CH₂CH₂NHMe |
| 142 | 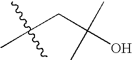 | Et | CH₂CH₂NHEt |
| 143 | 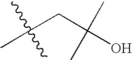 | Et | CH₂CH₂NHPr |
| 144 | 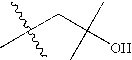 | Et | CH₂CH₂NH(i-Pr) |
| 145 | 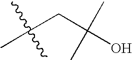 | Et | CH₂CH₂NMe₂ |
| 146 | 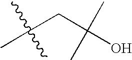 | Et | CH₂CH₂NEt₂ |
| 147 | 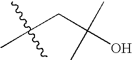 | Et | CH₂CH₂N(n-Pr)₂ |
| 148 | 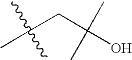 | Et | 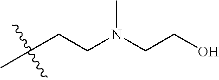 |
| 149 | 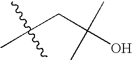 | Et | 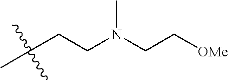 |
| 150 | 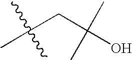 | Et | 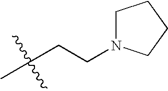 |
| 151 | 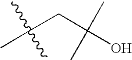 | Et | 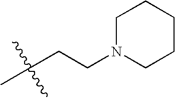 |
| 152 | 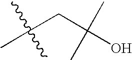 | Et | 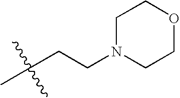 |
| 153 | 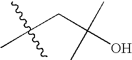 | 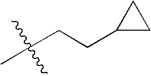 | CH₂CH₂NH₂ |
| 154 | 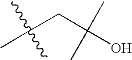 | 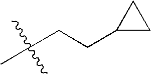 | CH₂CH₂NHMe |
| 155 | 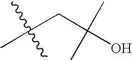 | 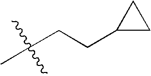 | CH₂CH₂NHEt |
| 156 | 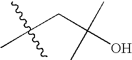 | 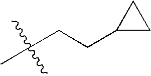 | CH₂CH₂NHPr |
| 157 | 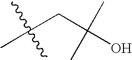 | 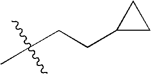 | CH₂CH₂NH(i-Pr) |
| 158 | 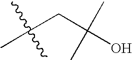 | 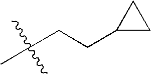 | CH₂CH₂NMe₂ |
| 159 | 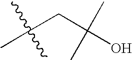 | 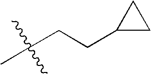 | CH₂CH₂NEt₂ |
| 160 | 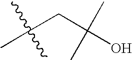 | 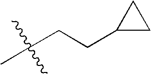 | CH₂CH₂N(n-Pr)₂ |
| 161 | 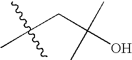 | 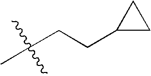 | 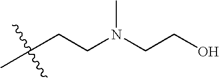 |
| 162 | 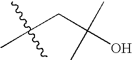 | 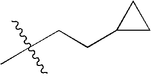 | 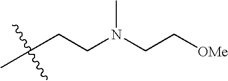 |
| 163 | 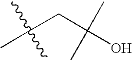 | 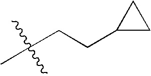 | 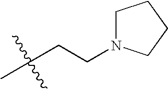 |
| 164 | 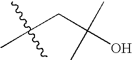 | 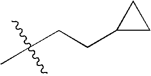 | 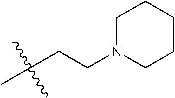 |
| 165 | 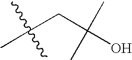 | 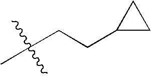 | 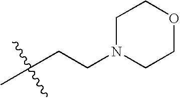 |
| 166 | 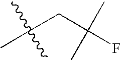 | Me | CH₂CH₂NH₂ |
| 167 | 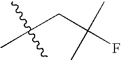 | Me | CH₂CH₂NHMe |
| 168 | 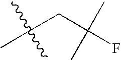 | Me | CH₂CH₂NHEt |
| 169 | 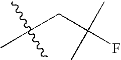 | Me | CH₂CH₂NHPr |
| 170 | 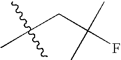 | Me | CH₂CH₂NH(i-Pr) |
| 171 | 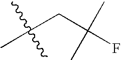 | Me | CH₂CH₂NMe₂ |
| 172 | 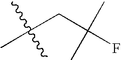 | Me | CH₂CH₂NEt₂ |
| 173 | 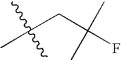 | Me | CH₂CH₂N(n-Pr)₂ |
| 174 | 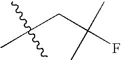 | Me | 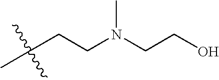 |
| 175 | 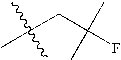 | Me | 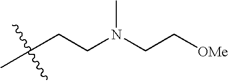 |
| 176 | 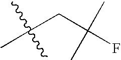 | Me | 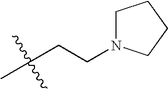 |
| 177 | 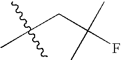 | Me | 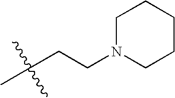 |
| 178 | 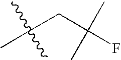 | Me | 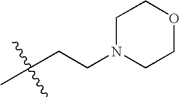 |
| 179 | 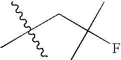 | Et | CH₂CH₂NH₂ |
| 180 | 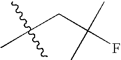 | Et | CH₂CH₂NHMe |
| 181 | 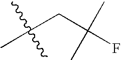 | Et | CH₂CH₂NHEt |
| 182 | 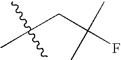 | Et | CH₂CH₂NHPr |
| 183 | 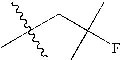 | Et | CH₂CH₂NH(i-Pr) |
| 184 | 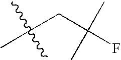 | Et | CH₂CH₂NMe₂ |
| 185 | 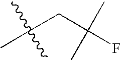 | Et | CH₂CH₂NEt₂ |
| 186 | 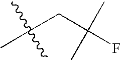 | Et | CH₂CH₂N(n-Pr)₂ |
| 187 | 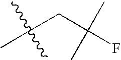 | Et | 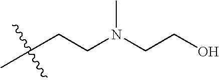 |
| 188 | 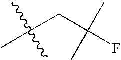 | Et | 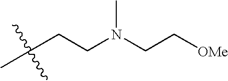 |
| 189 | 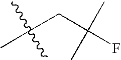 | Et | 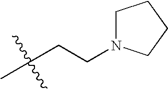 |
| 190 | 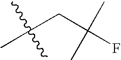 | Et | 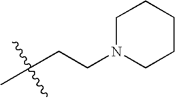 |
| 191 | 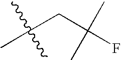 | Et | 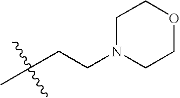 |
| 192 | 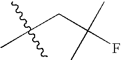 | 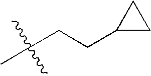 | CH₂CH₂NH₂ |
| 193 | 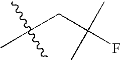 | 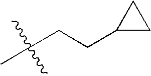 | CH₂CH₂NHMe |
| 194 | 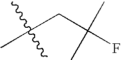 | 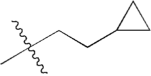 | CH₂CH₂NHEt |
| 195 | 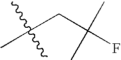 | 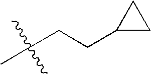 | CH₂CH₂NHPr |
| 196 | 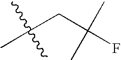 | 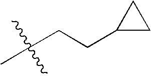 | CH₂CH₂NH(i-Pr) |
| 197 | 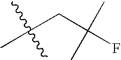 | 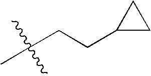 | CH₂CH₂NMe₂ |
| 198 | 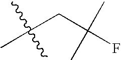 | 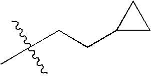 | CH₂CH₂NEt₂ |
| 199 | 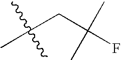 | 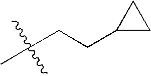 | CH₂CH₂N(n-Pr)₂ |
| 200 | 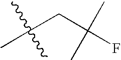 | 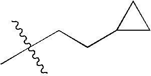 | 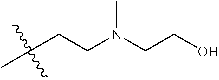 |
| 201 | 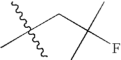 | 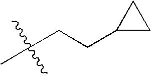 | 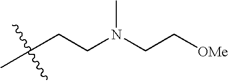 |
| 202 | 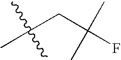 | 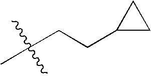 | 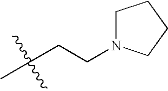 |
| 203 | 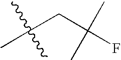 | 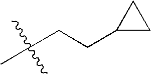 | 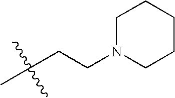 |
| 204 | 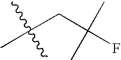 | 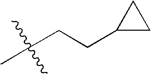 | 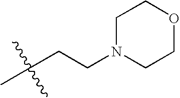 |

TABLE 2

| | |
|---|---|
| | Ib |
| 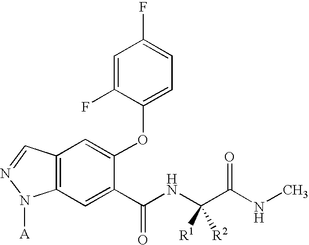 | |

| Example | | | |
|---|---|---|---|
| # | A | R¹ | R² |
| 205 | Me | Me | CH₂CH₂NH₂ |
| 206 | Me | Me | CH₂CH₂NHMe |
| 207 | Me | Me | CH₂CH₂NHEt |
| 208 | Me | Me | CH₂CH₂NHPr |
| 209 | Me | Me | CH₂CH₂NH(i-Pr) |
| 210 | Me | Me | CH₂CH₂NMe₂ |
| 211 | Me | Me | CH₂CH₂NEt₂ |
| 212 | Me | Me | CH₂CH₂N(n-Pr)₂ |
| 213 | Me | Me | 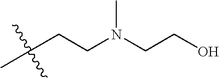 |
| 214 | Me | Me | 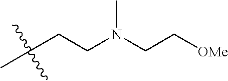 |
| 215 | Me | Me | 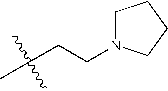 |
| 216 | Me | Me | 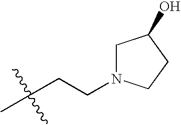 |
| 217 | Me | Me | 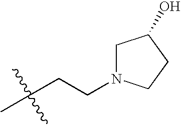 |
| 218 | Me | Me | 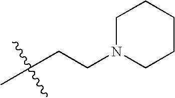 |
| 219 | Me | Me | 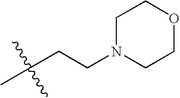 |
| 220 | Me | Et | CH₂CH₂NH₂ |
| 221 | Me | Et | CH₂CH₂NHMe |
| 222 | Me | Et | CH₂CH₂NHEt |
| 223 | Me | Et | CH₂CH₂NHPr |
| 224 | Me | Et | CH₂CH₂NH(i-Pr) |
| 225 | Me | Et | CH₂CH₂NMe₂ |
| 226 | Me | Et | CH₂CH₂NEt₂ |
| 227 | Me | Et | CH₂CH₂N(n-Pr)₂ |
| 228 | Me | Et | 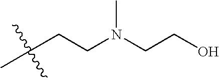 |
| 229 | Me | Et | 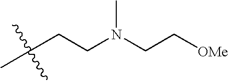 |
| 230 | Me | Et | 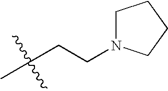 |
| 231 | Me | Et | 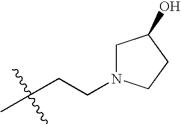 |
| 232 | Me | Et | 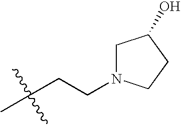 |
| 233 | Me | Et | 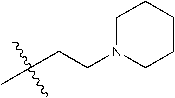 |
| 234 | Me | Et | 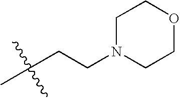 |
| 235 | Me | 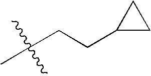 | CH₂CH₂NH₂ |
| 236 | Me | 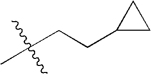 | CH₂CH₂NHMe |
| 237 | Me | 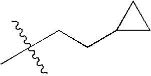 | CH₂CH₂NHEt |
| 238 | Me | 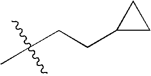 | CH₂CH₂NHPr |
| 239 | Me | 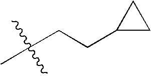 | CH₂CH₂NH(i-Pr) |
| 240 | Me | 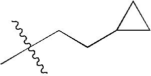 | CH₂CH₂NMe₂ |
| 241 | Me | 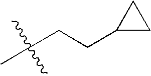 | CH₂CH₂NEt₂ |
| 242 | Me | 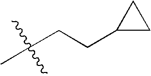 | CH₂CH₂N(n-Pr)₂ |
| 243 | Me | 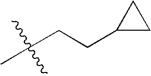 | 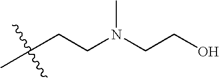 |
| 244 | Me | 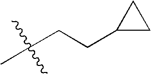 | 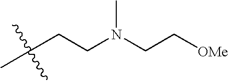 |
| 245 | Me | 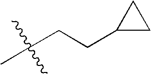 | 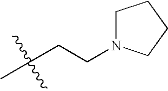 |
| 246 | Me | 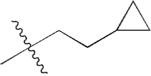 | 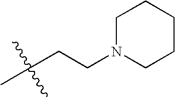 |
| 247 | Me | 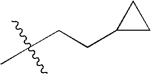 | 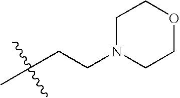 |
| 248 | 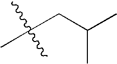 | Me | CH₂CH₂NH₂ |
| 249 | 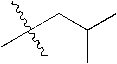 | Me | CH₂CH₂NHMe |
| 250 | 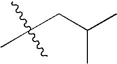 | Me | CH₂CH₂NHEt |
| 251 | 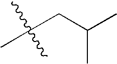 | Me | CH₂CH₂NHPr |
| 252 | 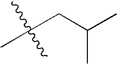 | Me | CH₂CH₂NH(i-Pr) |
| 253 | 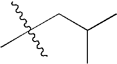 | Me | CH₂CH₂NMe₂ |
| 254 | 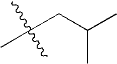 | Me | CH₂CH₂NEt₂ |
| 255 | 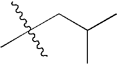 | Me | CH₂CH₂N(n-Pr)₂ |
| 256 | 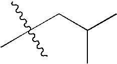 | Me | 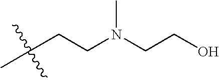 |
| 257 | 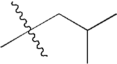 | Me | 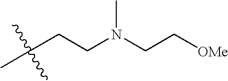 |
| 258 | 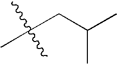 | Me | 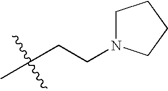 |
| 259 | 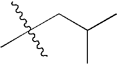 | Me | 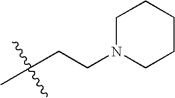 |
| 260 | 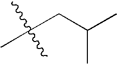 | Me | 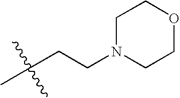 |
| 261 | 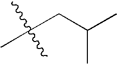 | Et | CH₂CH₂NH₂ |
| 262 | 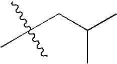 | Et | CH₂CH₂NHMe |
| 263 | 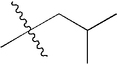 | Et | CH₂CH₂NHEt |
| 264 | 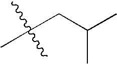 | Et | CH₂CH₂NHPr |
| 265 | 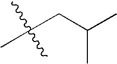 | Et | CH₂CH₂NH(i-Pr) |
| 266 | 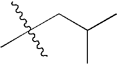 | Et | CH₂CH₂NMe₂ |
| 267 | 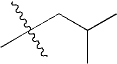 | Et | CH₂CH₂NEt₂ |
| 268 | 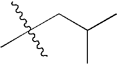 | Et | CH₂CH₂N(n-Pr)₂ |
| 269 | 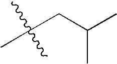 | Et | 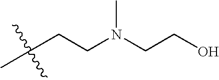 |
| 270 | 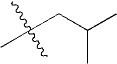 | Et | 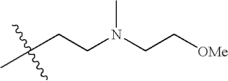 |
| 271 | 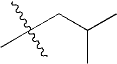 | Et | 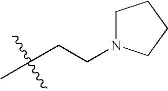 |
| 272 | 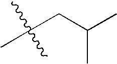 | Et | 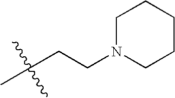 |
| 273 | 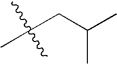 | Et | 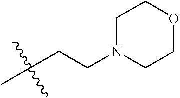 |
| 274 | 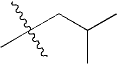 | 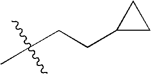 | CH₂CH₂NH₂ |
| 275 | 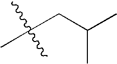 | 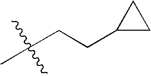 | CH₂CH₂NHMe |
| 276 | 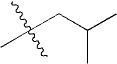 | 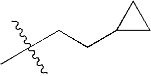 | CH₂CH₂NHEt |
| 277 | 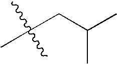 | 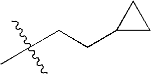 | CH₂CH₂NHPr |
| 278 | 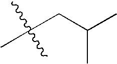 | 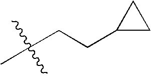 | CH₂CH₂NH(i-Pr) |
| 279 | 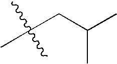 | 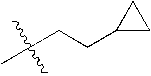 | CH₂CH₂NMe₂ |
| 280 | 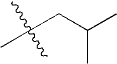 | 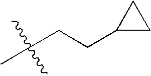 | CH₂CH₂NEt₂ |
| 281 | 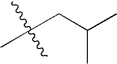 | 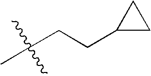 | CH₂CH₂N(n-Pr)₂ |
| 282 | 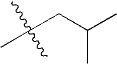 | 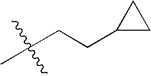 | 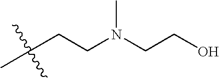 |
| 283 | 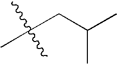 | 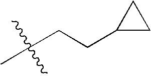 | 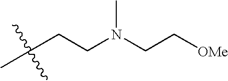 |
| 284 | 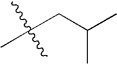 | 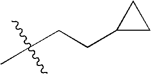 | 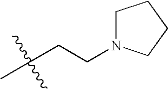 |
| 285 | 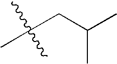 | 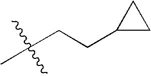 | 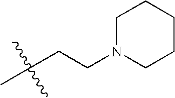 |
| 286 | 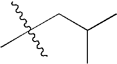 | 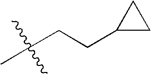 | 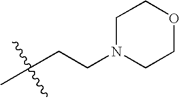 |
| 287 | 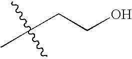 | Me | CH₂CH₂NH₂ |
| 288 | 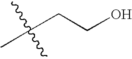 | Me | CH₂CH₂NHMe |
| 289 | 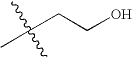 | Me | CH₂CH₂NHEt |
| 290 | 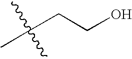 | Me | CH₂CH₂NHPr |
| 291 | 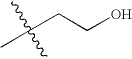 | Me | CH₂CH₂NH(i-Pr) |
| 292 | 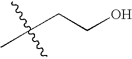 | Me | CH₂CH₂NMe₂ |
| 293 | 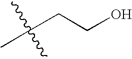 | Me | CH₂CH₂NEt₂ |
| 294 | 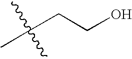 | Me | CH₂CH₂N(n-Pr)₂ |
| 295 | 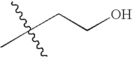 | Me | 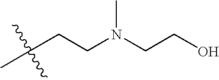 |
| 296 | 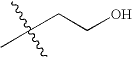 | Me | 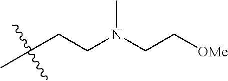 |
| 297 | 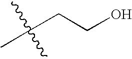 | Me | 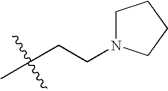 |
| 298 | 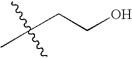 | Me | 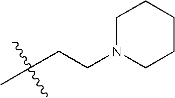 |
| 299 | 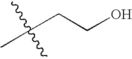 | Me | 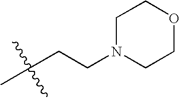 |
| 230 | 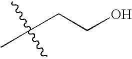 | Et | CH₂CH₂NH₂ |
| 231 | 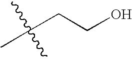 | Et | CH₂CH₂NHMe |
| 232 | 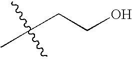 | Et | CH₂CH₂NHEt |
| 233 | 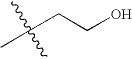 | Et | CH₂CH₂NHPr |
| 234 | 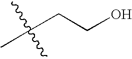 | Et | CH₂CH₂NH(i-Pr) |
| 235 | 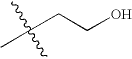 | Et | CH₂CH₂NMe₂ |
| 236 | 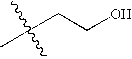 | Et | CH₂CH₂NEt₂ |
| 237 | 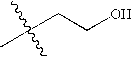 | Et | CH₂CH₂N(n-Pr)₂ |
| 238 | 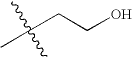 | Et | 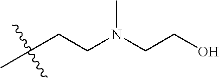 |
| 239 | 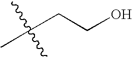 | Et | 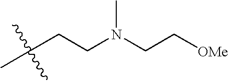 |
| 240 | 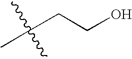 | Et | 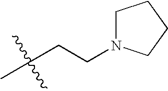 |
| 241 | 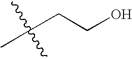 | Et | 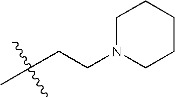 |
| 242 | 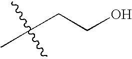 | Et | 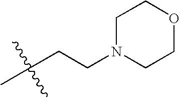 |
| 243 | 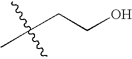 | 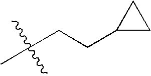 | CH₂CH₂NH₂ |
| 244 | 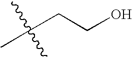 | 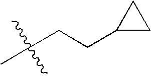 | CH₂CH₂NHMe |
| 245 | 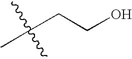 | 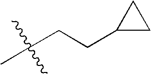 | CH₂CH₂NHEt |
| 246 | 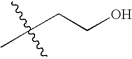 | 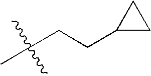 | CH₂CH₂NHPr |
| 247 | 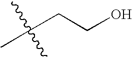 | 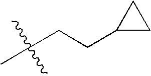 | CH₂CH₂NH(i-Pr) |
| 268 | 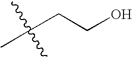 | 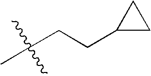 | CH₂CH₂NMe₂ |
| 249 | 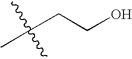 | 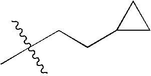 | CH₂CH₂NEt₂ |
| 250 | 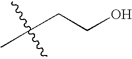 | 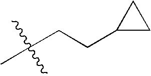 | CH₂CH₂N(n-Pr)₂ |
| 251 | 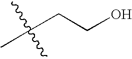 | 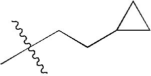 | 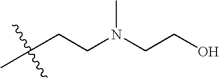 |
| 252 | 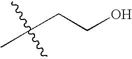 | 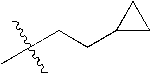 | 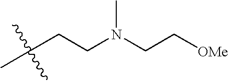 |
| 253 | 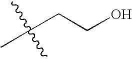 | 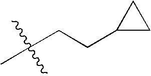 | 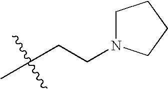 |
| 254 | 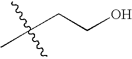 | 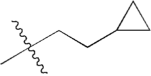 | 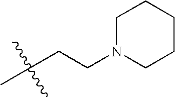 |
| 255 | 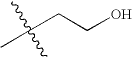 | 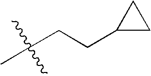 | 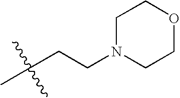 |
| 256 | 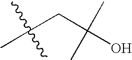 | Me | CH₂CH₂NH₂ |
| 257 | 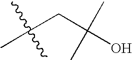 | Me | CH₂CH₂NHMe |
| 258 | 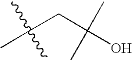 | Me | CH₂CH₂NHEt |
| 259 | 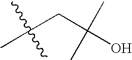 | Me | CH₂CH₂NHPr |
| 260 | 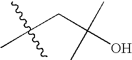 | Me | CH₂CH₂NH(i-Pr) |
| 261 | 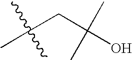 | Me | CH₂CH₂NMe₂ |
| 262 | 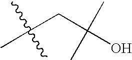 | Me | CH₂CH₂NEt₂ |
| 263 | 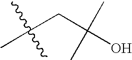 | Me | CH₂CH₂N(n-Pr)₂ |
| 264 | 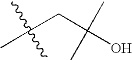 | Me | 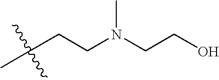 |
| 265 | 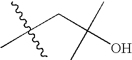 | Me | 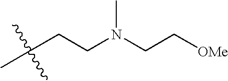 |
| 266 | 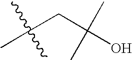 | Me | 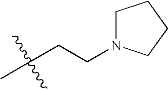 |
| 267 | 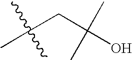 | Me | 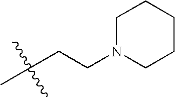 |
| 268 | 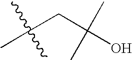 | Me | 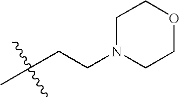 |
| 269 | 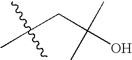 | Et | CH₂CH₂NH₂ |
| 270 | 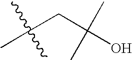 | Et | CH₂CH₂NHMe |
| 271 | 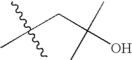 | Et | CH₂CH₂NHEt |
| 272 | 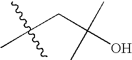 | Et | CH₂CH₂NHPr |
| 273 | 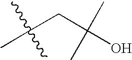 | Et | CH₂CH₂NH(i-Pr) |
| 274 | 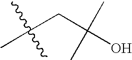 | Et | CH₂CH₂NMe₂ |
| 275 | 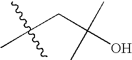 | Et | CH₂CH₂NEt₂ |
| 276 | 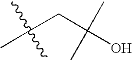 | Et | CH₂CH₂N(n-Pr)₂ |
| 277 | 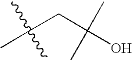 | Et | 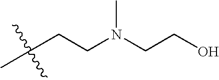 |
| 278 | 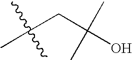 | Et | 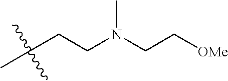 |
| 279 | 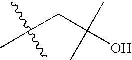 | Et | 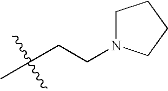 |
| 280 | 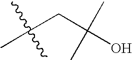 | Et | 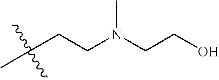 |
| 281 | 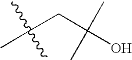 | Et | 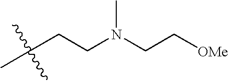 |
| 282 | 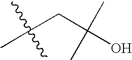 | 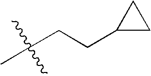 | CH₂CH₂NH₂ |
| 283 | 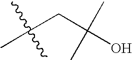 | 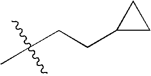 | CH₂CH₂NHMe |
| 284 | 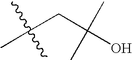 | 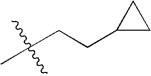 | CH₂CH₂NHEt |
| 285 | 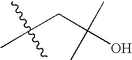 | 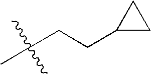 | CH₂CH₂NHPr |
| 286 | 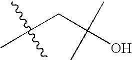 | 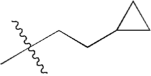 | CH₂CH₂NH(i-Pr) |
| 287 | 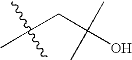 | 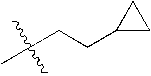 | CH₂CH₂NMe₂ |
| 288 | 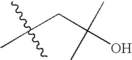 | 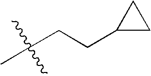 | CH₂CH₂NEt₂ |
| 289 | 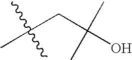 | 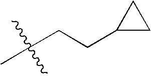 | CH₂CH₂N(n-Pr)₂ |
| 290 | 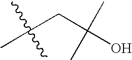 | 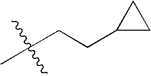 | 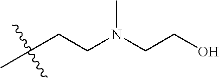 |
| 291 | 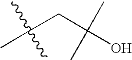 | 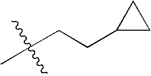 | 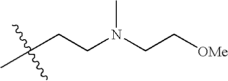 |
| 292 | 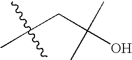 | 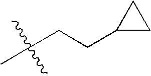 | 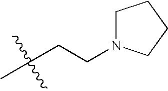 |
| 293 | 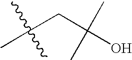 | 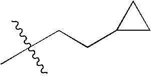 | 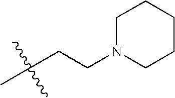 |
| 294 | 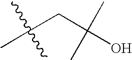 | 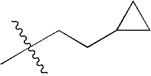 | 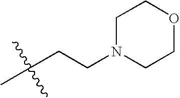 |
| 295 | 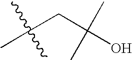 | Me | CH₂CH₂NH₂ |
| 296 | 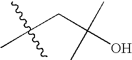 | Me | CH₂CH₂NHMe |
| 297 | 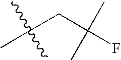 | Me | CH₂CH₂NHEt |
| 298 | 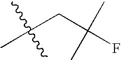 | Me | CH₂CH₂NHPr |
| 299 | 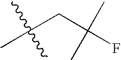 | Me | CH₂CH₂NH(i-Pr) |
| 300 | 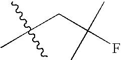 | Me | CH₂CH₂NMe₂ |
| 301 | 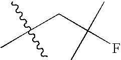 | Me | CH₂CH₂NEt₂ |
| 302 | 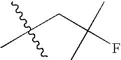 | Me | CH₂CH₂N(n-Pr)₂ |
| 303 | 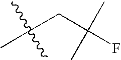 | Me | 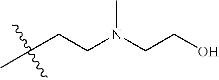 |
| 304 | 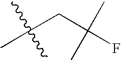 | Me | 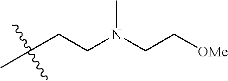 |
| 305 | 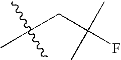 | Me | 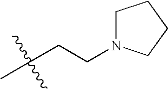 |
| 306 | 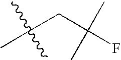 | Me | 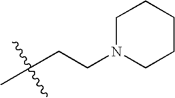 |
| 307 | 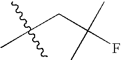 | Me | 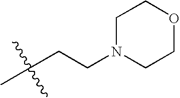 |
| 308 | 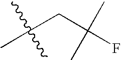 | Et | CH₂CH₂NH₂ |
| 309 | 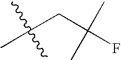 | Et | CH₂CH₂NHMe |
| 310 | 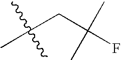 | Et | CH₂CH₂NHEt |
| 311 | 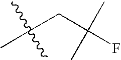 | Et | CH₂CH₂NHPr |
| 312 | 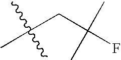 | Et | CH₂CH₂NH(i-Pr) |
| 313 | 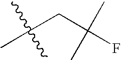 | Et | CH₂CH₂NMe₂ |
| 314 | 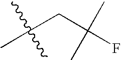 | Et | CH₂CH₂NEt₂ |
| 315 | 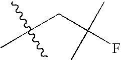 | Et | CH₂CH₂N(n-Pr)₂ |
| 316 | 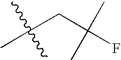 | Et | 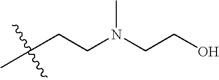 |
| 317 | 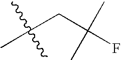 | Et | 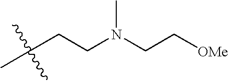 |
| 318 | 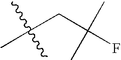 | Et | 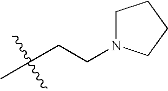 |
| 319 | 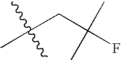 | Et | 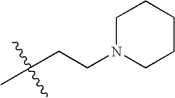 |
| 320 | 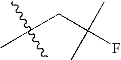 | Et | 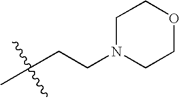 |
| 321 | 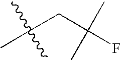 | 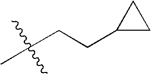 | CH₂CH₂NH₂ |
| 322 | 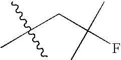 | 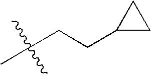 | CH₂CH₂NHMe |
| 323 | 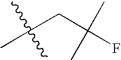 | 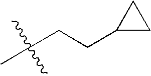 | CH₂CH₂NHEt |
| 324 | 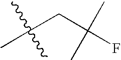 | 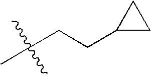 | CH₂CH₂NHPr |
| 325 | 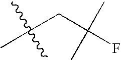 | 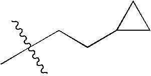 | CH₂CH₂NH(i-Pr) |
| 326 | 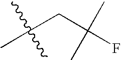 | 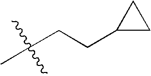 | CH₂CH₂NMe₂ |
| 327 | 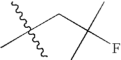 | 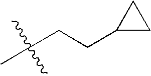 | CH₂CH₂NEt₂ |
| 328 | 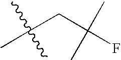 | 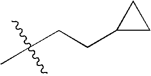 | CH₂CH₂N(n-Pr)₂ |
| 329 | 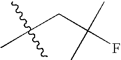 | 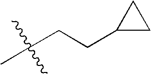 | 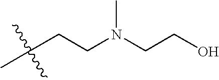 |
| 330 | 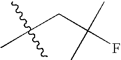 | 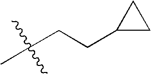 | 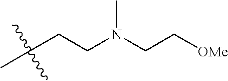 |
| 331 | 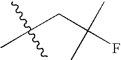 | 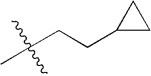 | 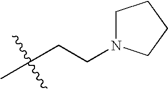 |
| 332 | 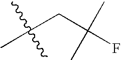 | 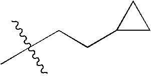 | 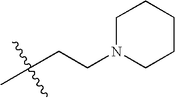 |
| 333 | 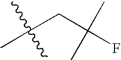 | 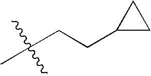 | 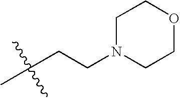 |

TABLE 3

| | |
|---|---|
| | Ic |
| 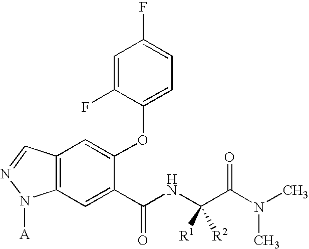 | |

| Example | | | |
|---|---|---|---|
| # | A | R¹ | R² |
| 334 | Me | Me | CH₂CH₂NH₂ |
| 335 | Me | Me | CH₂CH₂NHMe |
| 336 | Me | Me | CH₂CH₂NHEt |
| 337 | Me | Me | CH₂CH₂NHPr |
| 338 | Me | Me | CH₂CH₂NH(i-Pr) |
| 339 | Me | Me | CH₂CH₂NMe₂ |
| 340 | Me | Me | CH₂CH₂NEt₂ |
| 341 | Me | Me | CH₂CH₂N(n-Pr)₂ |
| 342 | Me | Me | 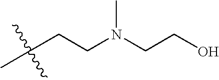 |
| 343 | Me | Me | 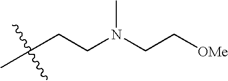 |
| 344 | Me | Me | 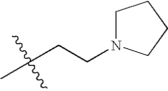 |
| 345 | Me | Me | 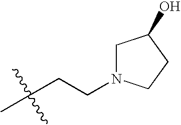 |
| 346 | Me | Me | 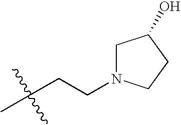 |
| 347 | Me | Me | 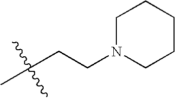 |
| 348 | Me | Me | 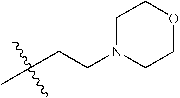 |
| 349 | Me | Et | CH₂CH₂NH₂ |
| 350 | Me | Et | CH₂CH₂NHMe |
| 351 | Me | Et | CH₂CH₂NHEt |
| 352 | Me | Et | CH₂CH₂NHPr |
| 353 | Me | Et | CH₂CH₂NH(i-Pr) |
| 354 | Me | Et | CH₂CH₂NMe₂ |
| 355 | Me | Et | CH₂CH₂NEt₂ |
| 356 | Me | Et | CH₂CH₂N(n-Pr)₂ |
| 357 | Me | Et | 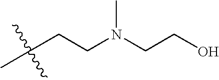 |
| 358 | Me | Et | 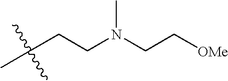 |
| 359 | Me | Et | 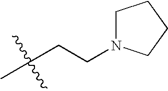 |
| 360 | Me | Et | 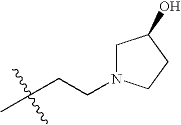 |
| 361 | Me | Et | 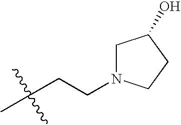 |
| 362 | Me | Et | 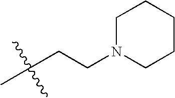 |
| 363 | Me | Et | 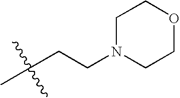 |
| 364 | Me | 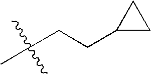 | CH₂CH₂NH₂ |
| 365 | Me | 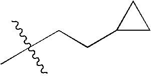 | CH₂CH₂NHMe |
| 366 | Me | 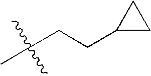 | CH₂CH₂NHEt |
| 367 | Me | 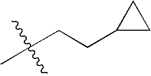 | CH₂CH₂NHPr |
| 368 | Me | 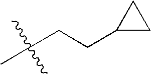 | CH₂CH₂NH(i-Pr) |
| 369 | Me | 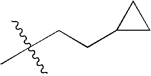 | CH₂CH₂NMe₂ |
| 370 | Me | 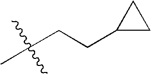 | CH₂CH₂NEt₂ |
| 371 | Me | 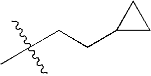 | CH₂CH₂N(n-Pr)₂ |
| 372 | Me | 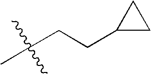 | 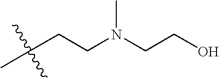 |
| 373 | Me | 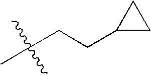 | 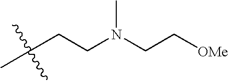 |
| 374 | Me | 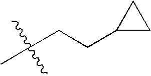 | 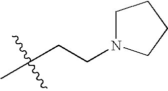 |
| 375 | Me | 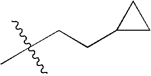 | 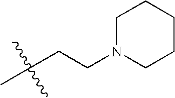 |
| 376 | Me | 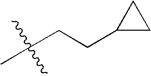 | 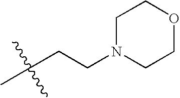 |
| 377 | 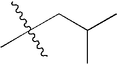 | Me | CH₂CH₂NH₂ |
| 378 | 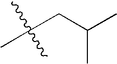 | Me | CH₂CH₂NHMe |
| 379 | 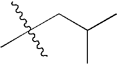 | Me | CH₂CH₂NHEt |
| 380 | 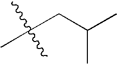 | Me | CH₂CH₂NHPr |
| 381 | 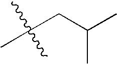 | Me | CH₂CH₂NH(i-Pr) |
| 382 | 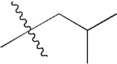 | Me | CH₂CH₂NMe₂ |
| 383 | 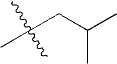 | Me | CH₂CH₂NEt₂ |
| 384 | 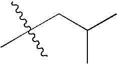 | Me | CH₂CH₂N(n-Pr)₂ |
| 385 | 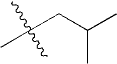 | Me | 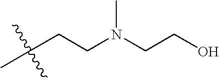 |
| 386 | 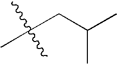 | Me | 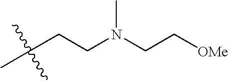 |
| 387 | 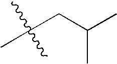 | Me | 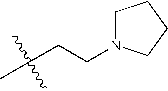 |
| 388 | 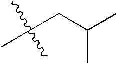 | Me | 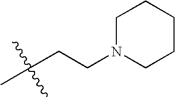 |
| 389 | 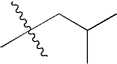 | Me | 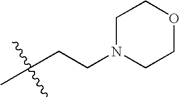 |
| 390 | 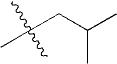 | Et | CH₂CH₂NH₂ |
| 391 | 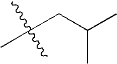 | Et | CH₂CH₂NHMe |
| 392 | 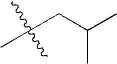 | Et | CH₂CH₂NHEt |
| 393 | 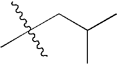 | Et | CH₂CH₂NHPr |
| 394 | 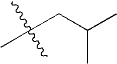 | Et | CH₂CH₂NH(i-Pr) |
| 395 | 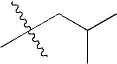 | Et | CH₂CH₂NMe₂ |
| 396 | 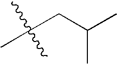 | Et | CH₂CH₂NEt₂ |
| 397 | 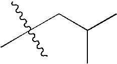 | Et | CH₂CH₂N(n-Pr)₂ |
| 398 | 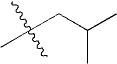 | Et | 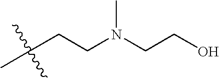 |
| 399 | 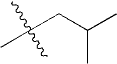 | Et | 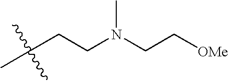 |
| 400 | 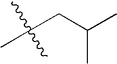 | Et | 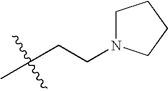 |
| 401 | 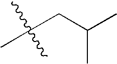 | Et | 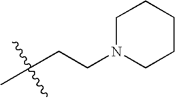 |
| 402 | 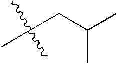 | Et | 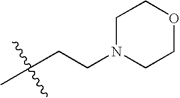 |
| 403 | 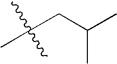 | 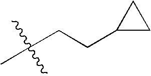 | CH₂CH₂NH₂ |
| 404 | 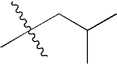 | 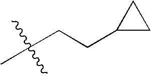 | CH₂CH₂NHMe |
| 405 | 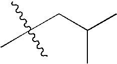 | 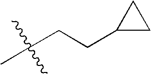 | CH₂CH₂NHEt |
| 406 | 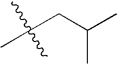 | 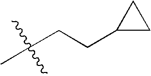 | CH₂CH₂NHPr |
| 407 | 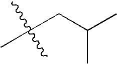 | 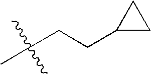 | CH₂CH₂NH(i-Pr) |
| 408 | 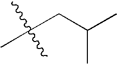 | 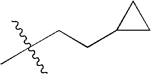 | CH₂CH₂NMe₂ |
| 409 | 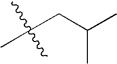 | 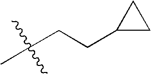 | CH₂CH₂NEt₂ |
| 410 | 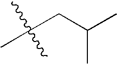 | 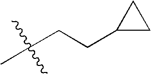 | CH₂CH₂N(n-Pr)₂ |
| 411 | 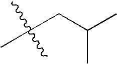 | 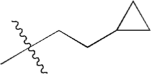 | 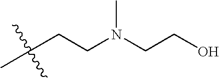 |
| 412 | 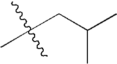 | 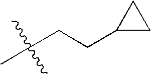 | 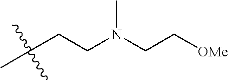 |
| 413 | 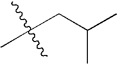 | 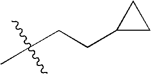 | 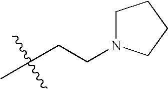 |
| 414 | 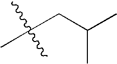 | 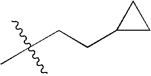 | 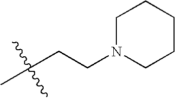 |
| 415 | 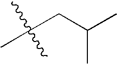 | 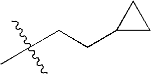 | 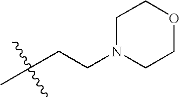 |
| 416 | 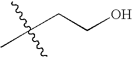 | Me | CH₂CH₂NH₂ |
| 417 | 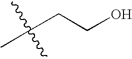 | Me | CH₂CH₂NHMe |
| 418 | 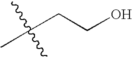 | Me | CH₂CH₂NHEt |
| 419 | 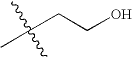 | Me | CH₂CH₂NHPr |
| 420 | 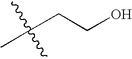 | Me | CH₂CH₂NH(i-Pr) |
| 421 | 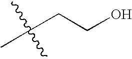 | Me | CH₂CH₂NMe₂ |
| 422 | 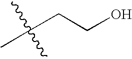 | Me | CH₂CH₂NEt₂ |
| 423 | 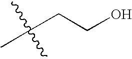 | Me | CH₂CH₂N(n-Pr)₂ |
| 424 | 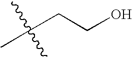 | Me | 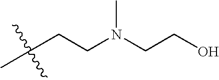 |
| 425 | 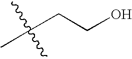 | Me | 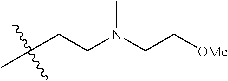 |
| 426 | 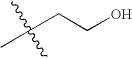 | Me | 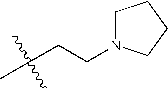 |
| 427 | 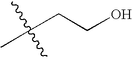 | Me | 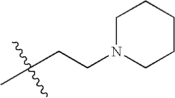 |
| 428 | 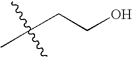 | Me | 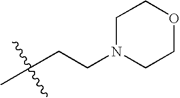 |
| 429 | 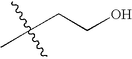 | Et | CH₂CH₂NH₂ |
| 430 | 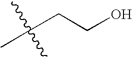 | Et | CH₂CH₂NHMe |
| 431 | 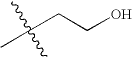 | Et | CH₂CH₂NHEt |
| 432 | 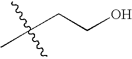 | Et | CH₂CH₂NHPr |
| 433 | 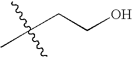 | Et | CH₂CH₂NH(i-Pr) |
| 434 | 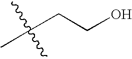 | Et | CH₂CH₂NMe₂ |
| 435 | 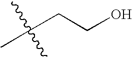 | Et | CH₂CH₂NEt₂ |
| 436 | 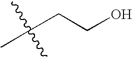 | Et | CH₂CH₂N(n-Pr)₂ |
| 437 | 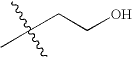 | Et | 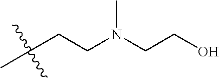 |
| 438 | 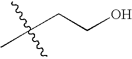 | Et | 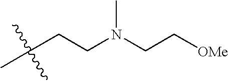 |
| 439 | 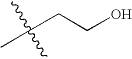 | Et | 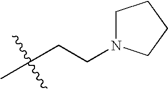 |
| 440 | 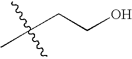 | Et | 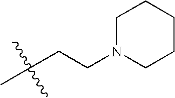 |
| 441 | 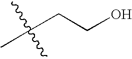 | Et | 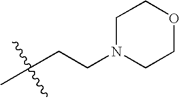 |
| 442 | 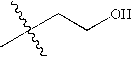 | 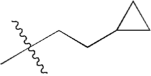 | CH₂CH₂NH₂ |
| 443 | 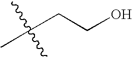 | 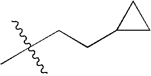 | CH₂CH₂NHMe |
| 444 | 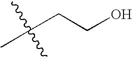 | 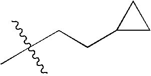 | CH₂CH₂NHEt |
| 445 | 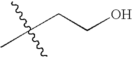 | 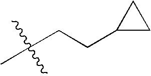 | CH₂CH₂NHPr |
| 446 | 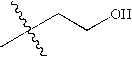 | 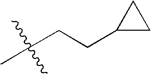 | CH₂CH₂NH(i-Pr) |
| 447 | 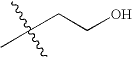 | 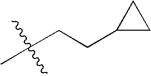 | CH₂CH₂NMe₂ |
| 448 | 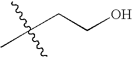 | 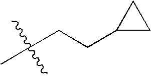 | CH₂CH₂NEt₂ |
| 449 | 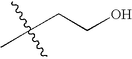 | 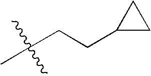 | CH₂CH₂N(n-Pr)₂ |
| 500 | 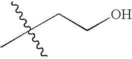 | 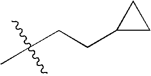 | 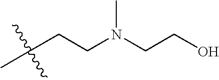 |
| 501 | 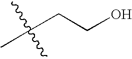 | 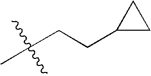 | 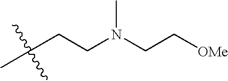 |
| 502 | 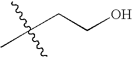 | 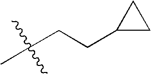 | 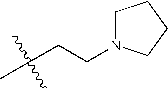 |
| 503 | 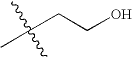 | 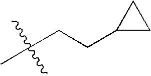 | 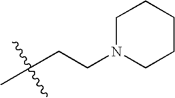 |
| 504 | 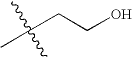 | 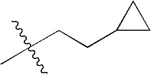 | 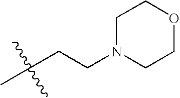 |
| 505 | 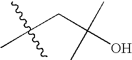 | Me | CH₂CH₂NH₂ |
| 506 | 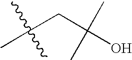 | Me | CH₂CH₂NHMe |
| 507 | 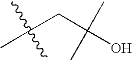 | Me | CH₂CH₂NHEt |
| 508 | 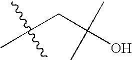 | Me | CH₂CH₂NHPr |
| 509 | 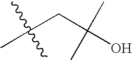 | Me | CH₂CH₂NH(i-Pr) |
| 510 | 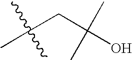 | Me | CH₂CH₂NMe₂ |
| 511 | 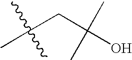 | Me | CH₂CH₂NEt₂ |
| 512 | 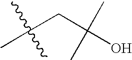 | Me | CH₂CH₂N(n-Pr)₂ |
| 513 | 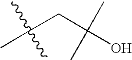 | Me | 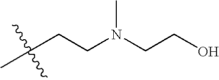 |
| 514 | 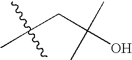 | Me | 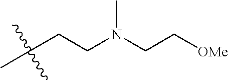 |
| 515 | 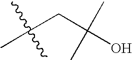 | Me | 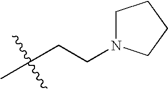 |
| 516 | 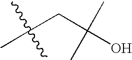 | Me | 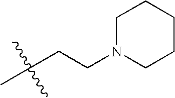 |
| 517 | 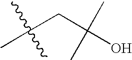 | Me | 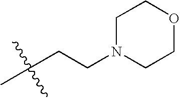 |
| 518 | 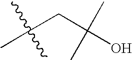 | Et | CH₂CH₂NH₂ |
| 519 | 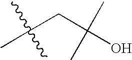 | Et | CH₂CH₂NHMe |
| 520 | 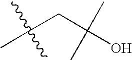 | Et | CH₂CH₂NHEt |
| 521 | 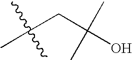 | Et | CH₂CH₂NHPr |
| 522 | 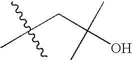 | Et | CH₂CH₂NH(i-Pr) |
| 523 | 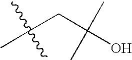 | Et | CH₂CH₂NMe₂ |
| 524 | 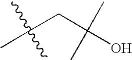 | Et | CH₂CH₂NEt₂ |
| 525 | 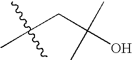 | Et | CH₂CH₂N(n-Pr)₂ |
| 526 | 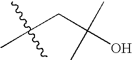 | Et | 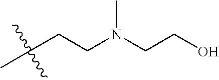 |
| 527 | 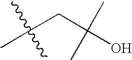 | Et | 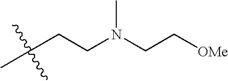 |
| 528 | 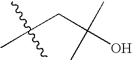 | Et | 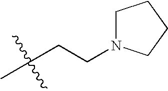 |
| 529 | 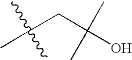 | Et | 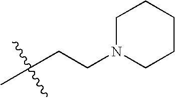 |
| 430 | 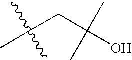 | Et | 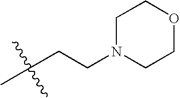 |
| 531 | 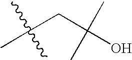 | 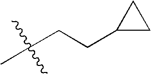 | CH₂CH₂NH₂ |
| 532 | 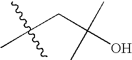 | 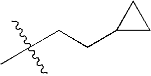 | CH₂CH₂NHMe |
| 533 | 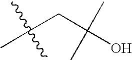 | 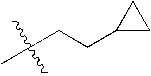 | CH₂CH₂NHEt |
| 534 | 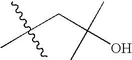 | 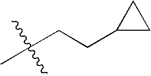 | CH₂CH₂NHPr |
| 535 | 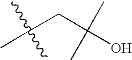 | 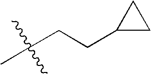 | CH₂CH₂NH(i-Pr) |
| 536 | 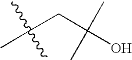 | 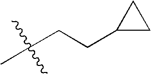 | CH₂CH₂NMe₂ |
| 537 | 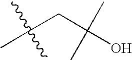 | 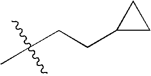 | CH₂CH₂NEt₂ |
| 538 | 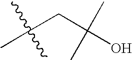 | 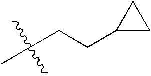 | CH₂CH₂N(n-Pr)₂ |
| 539 | 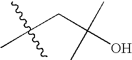 | 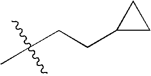 | 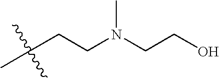 |
| 540 | 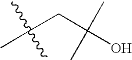 | 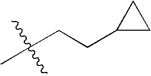 | 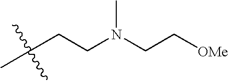 |
| 541 | 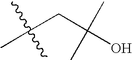 | 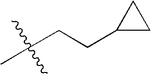 | 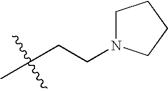 |
| 542 | 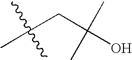 | 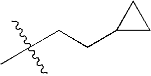 | 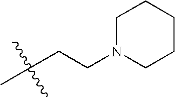 |
| 543 | 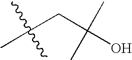 | 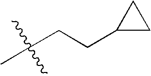 | 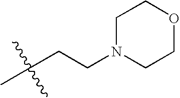 |
| 544 | 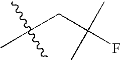 | Me | CH₂CH₂NH₂ |
| 545 | 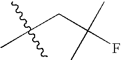 | Me | CH₂CH₂NHMe |
| 546 | 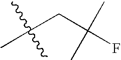 | Me | CH₂CH₂NHEt |
| 547 | 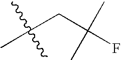 | Me | CH₂CH₂NHPr |
| 548 | 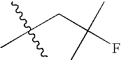 | Me | CH₂CH₂NH(i-Pr) |
| 549 | 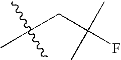 | Me | CH₂CH₂NMe₂ |
| 550 | 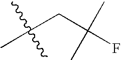 | Me | CH₂CH₂NEt₂ |
| 551 | 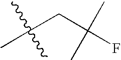 | Me | CH₂CH₂N(n-Pr)₂ |
| 552 | 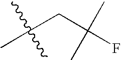 | Me | 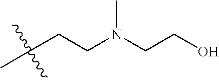 |
| 553 | 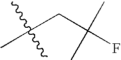 | Me | 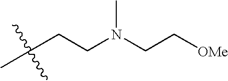 |
| 554 | 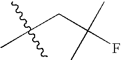 | Me | 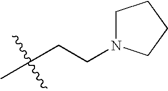 |
| 555 | 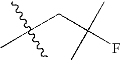 | Me | 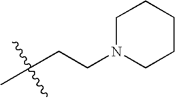 |
| 556 | 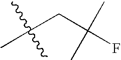 | Me | 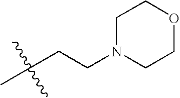 |
| 557 | 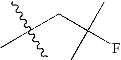 | Et | CH₂CH₂NH₂ |
| 558 | 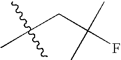 | Et | CH₂CH₂NHMe |
| 559 | 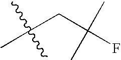 | Et | CH₂CH₂NHEt |
| 560 | 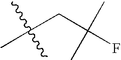 | Et | CH₂CH₂NHPr |
| 561 | 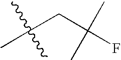 | Et | CH₂CH₂NH(i-Pr) |
| 562 | 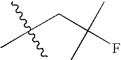 | Et | CH₂CH₂NMe₂ |
| 563 | 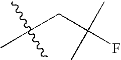 | Et | CH₂CH₂NEt₂ |
| 564 | 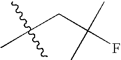 | Et | CH₂CH₂N(n-Pr)₂ |
| 565 | 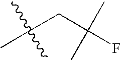 | Et | 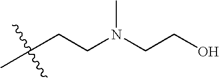 |
| 566 | 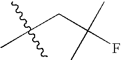 | Et | 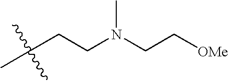 |
| 567 | 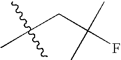 | Et | 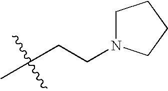 |
| 568 | 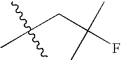 | Et | 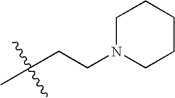 |
| 569 | 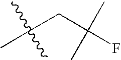 | Et | 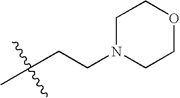 |
| 570 | 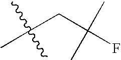 | 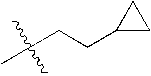 | CH₂CH₂NH₂ |
| 571 | 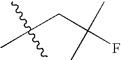 | 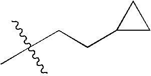 | CH₂CH₂NHMe |
| 572 | 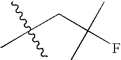 | 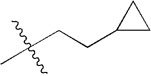 | CH₂CH₂NHEt |
| 573 | 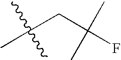 | 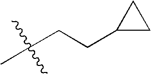 | CH₂CH₂NHPr |
| 574 | 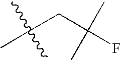 | 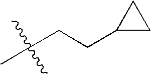 | CH₂CH₂NH(i-Pr) |
| 575 | 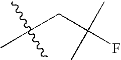 | 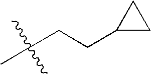 | CH₂CH₂NMe₂ |
| 576 | 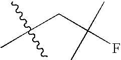 | 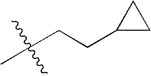 | CH₂CH₂NEt₂ |
| 577 | 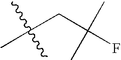 | 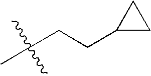 | CH₂CH₂N(n-Pr)₂ |
| 578 | 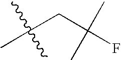 | 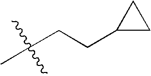 | 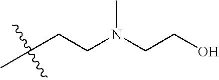 |
| 579 | 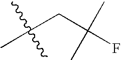 | 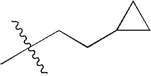 | 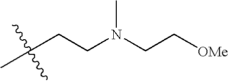 |
| 580 | 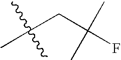 | 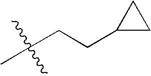 | 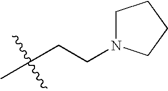 |
| 581 | 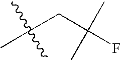 | 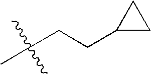 | 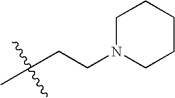 |
| 582 | 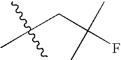 | 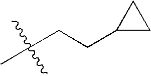 | 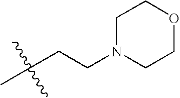 |

TABLE 4

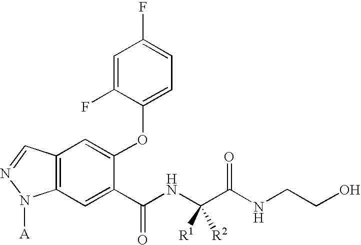

Id

| Example | | | |
|---|---|---|---|
| # | A | R¹ | R² |
| 583 | Me | Me | CH₂CH₂NH₂ |
| 584 | Me | Me | CH₂CH₂NHMe |
| 585 | Me | Me | CH₂CH₂NHEt |
| 586 | Me | Me | CH₂CH₂NHPr |
| 587 | Me | Me | CH₂CH₂NH(i-Pr) |
| 588 | Me | Me | CH₂CH₂NMe₂ |
| 589 | Me | Me | CH₂CH₂NEt₂ |
| 590 | Me | Me | CH₂CH₂N(n-Pr)₂ |
| 591 | Me | Me | 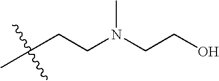 |
| 592 | Me | Me | 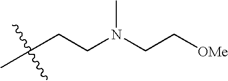 |
| 593 | Me | Me | 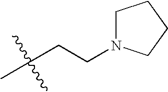 |
| 594 | Me | Me | 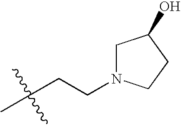 |
| 595 | Me | Me | 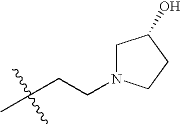 |
| 596 | Me | Me | 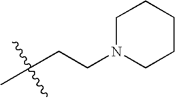 |
| 597 | Me | Me | 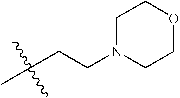 |
| 598 | Me | Et | CH₂CH₂NH₂ |
| 599 | Me | Et | CH₂CH₂NHMe |
| 600 | Me | Et | CH₂CH₂NHEt |
| 601 | Me | Et | CH₂CH₂NHPr |
| 602 | Me | Et | CH₂CH₂NH(i-Pr) |
| 603 | Me | Et | CH₂CH₂NMe₂ |
| 604 | Me | Et | CH₂CH₂NEt₂ |
| 605 | Me | Et | CH₂CH₂N(n-Pr)₂ |
| 606 | Me | Et | 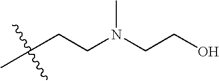 |
| 607 | Me | Et | 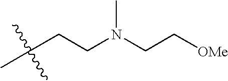 |
| 608 | Me | Et | 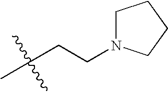 |
| 609 | Me | Et | 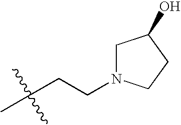 |
| 610 | Me | Et | 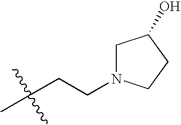 |
| 611 | Me | Et | 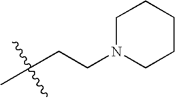 |
| 612 | Me | Et | 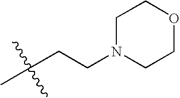 |
| 613 | Me | 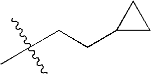 | CH₂CH₂NH₂ |
| 614 | Me | 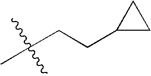 | CH₂CH₂NHMe |
| 615 | Me | 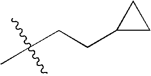 | CH₂CH₂NHEt |
| 616 | Me | 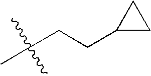 | CH₂CH₂NHPr |
| 617 | Me | 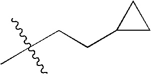 | CH₂CH₂NH(i-Pr) |
| 618 | Me | 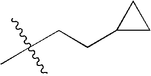 | CH₂CH₂NMe₂ |
| 619 | Me | 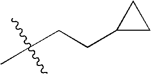 | CH₂CH₂NEt₂ |
| 620 | Me | 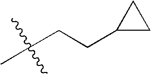 | CH₂CH₂N(n-Pr)₂ |
| 621 | Me | 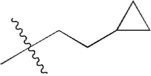 | 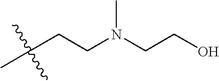 |
| 622 | Me | 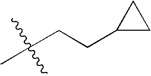 | 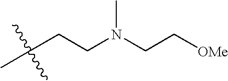 |
| 623 | Me | 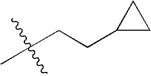 | 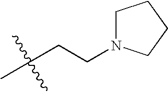 |
| 624 | Me | 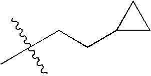 | 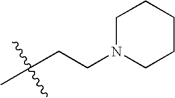 |
| 625 | Me | 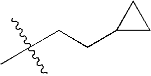 | 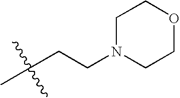 |
| 626 | 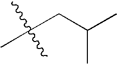 | Me | CH₂CH₂NH₂ |
| 627 | 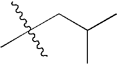 | Me | CH₂CH₂NHMe |
| 628 | 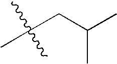 | Me | CH₂CH₂NHEt |
| 629 | 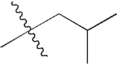 | Me | CH₂CH₂NHPr |
| 630 | 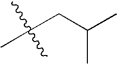 | Me | CH₂CH₂NH(i-Pr) |
| 631 | 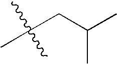 | Me | CH₂CH₂NMe₂ |
| 632 | 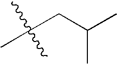 | Me | CH₂CH₂NEt₂ |
| 633 | 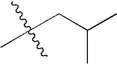 | Me | CH₂CH₂N(n-Pr)₂ |
| 634 | 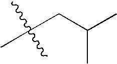 | Me | 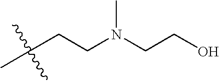 |
| 635 | 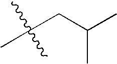 | Me | 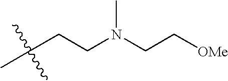 |
| 636 | 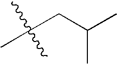 | Me | 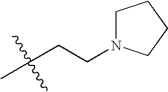 |
| 637 | 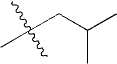 | Me | 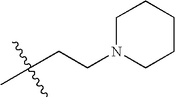 |
| 638 | 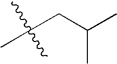 | Me | 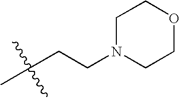 |
| 639 | 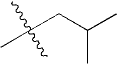 | Et | CH₂CH₂NH₂ |
| 640 | 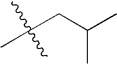 | Et | CH₂CH₂NHMe |
| 641 | 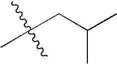 | Et | CH₂CH₂NHEt |
| 642 | 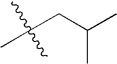 | Et | CH₂CH₂NHPr |
| 643 | 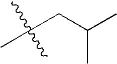 | Et | CH₂CH₂NH(i-Pr) |
| 644 | 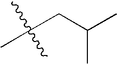 | Et | CH₂CH₂NMe₂ |
| 645 | 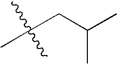 | Et | CH₂CH₂NEt₂ |
| 646 | 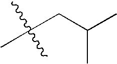 | Et | CH₂CH₂N(n-Pr)₂ |
| 647 | 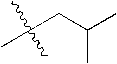 | Et | 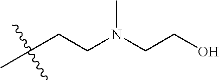 |
| 648 | 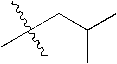 | Et | 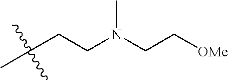 |
| 649 | 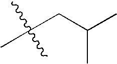 | Et | 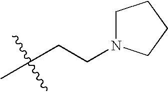 |
| 650 | 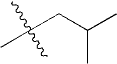 | Et | 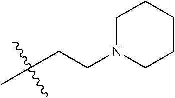 |
| 651 | 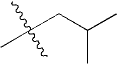 | Et | 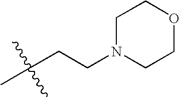 |
| 652 | 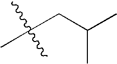 | 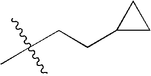 | CH₂CH₂NH₂ |
| 653 | 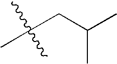 | 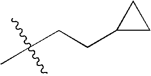 | CH₂CH₂NHMe |
| 654 | 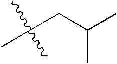 | 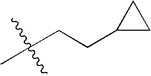 | CH₂CH₂NHEt |
| 655 | 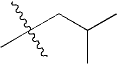 | 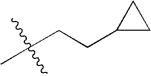 | CH₂CH₂NHPr |
| 656 | 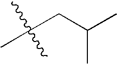 | 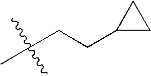 | CH₂CH₂NH(i-Pr) |
| 657 | 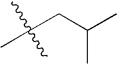 | 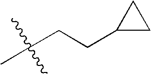 | CH₂CH₂NMe₂ |
| 658 | 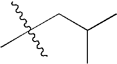 | 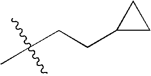 | CH₂CH₂NEt₂ |
| 659 | 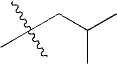 | 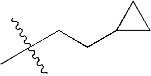 | CH₂CH₂N(n-Pr)₂ |
| 660 | 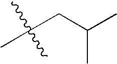 | 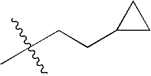 | 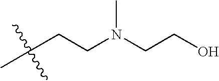 |
| 661 | 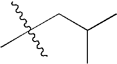 | 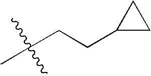 | 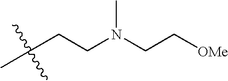 |
| 662 | 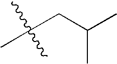 | 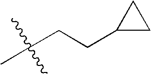 | 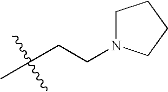 |
| 663 | 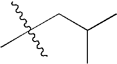 | 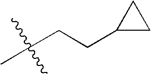 | 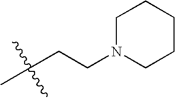 |
| 664 | 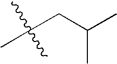 | 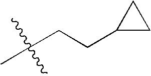 | 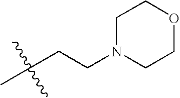 |
| 665 | 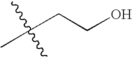 | Me | CH₂CH₂NH₂ |
| 666 | 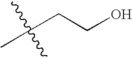 | Me | CH₂CH₂NHMe |
| 667 | 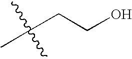 | Me | CH₂CH₂NHEt |
| 668 | 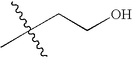 | Me | CH₂CH₂NHPr |
| 669 | 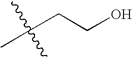 | Me | CH₂CH₂NH(i-Pr) |
| 670 | 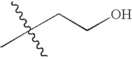 | Me | CH₂CH₂NMe₂ |
| 671 | 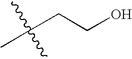 | Me | CH₂CH₂NEt₂ |
| 672 | 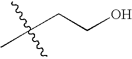 | Me | CH₂CH₂N(n-Pr)₂ |
| 673 | 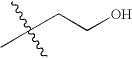 | Me | 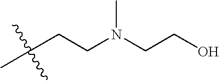 |
| 674 | 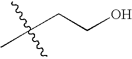 | Me | 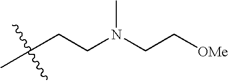 |
| 675 | 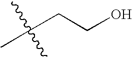 | Me | 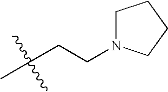 |
| 676 | 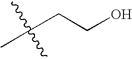 | Me | 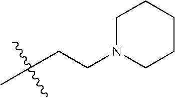 |
| 677 | 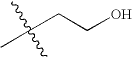 | Me | 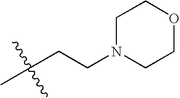 |
| 678 | 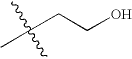 | Et | CH₂CH₂NH₂ |
| 679 | 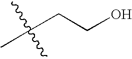 | Et | CH₂CH₂NHMe |
| 680 | 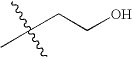 | Et | CH₂CH₂NHEt |
| 681 | 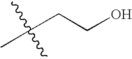 | Et | CH₂CH₂NHPr |
| 682 | 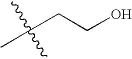 | Et | CH₂CH₂NH(i-Pr) |
| 683 | 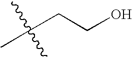 | Et | CH₂CH₂NMe₂ |
| 684 | 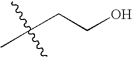 | Et | CH₂CH₂NEt₂ |
| 684 | 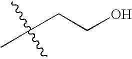 | Et | CH₂CH₂N(n-Pr)₂ |
| 686 | 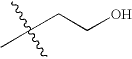 | Et | 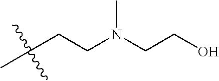 |
| 687 | 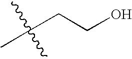 | Et | 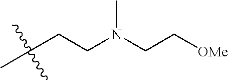 |
| 688 | 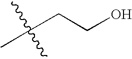 | Et | 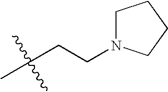 |
| 689 | 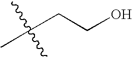 | Et | 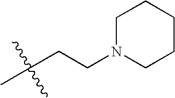 |
| 690 | 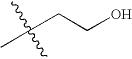 | Et | 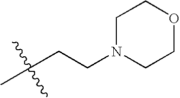 |
| 691 | 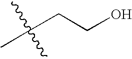 | 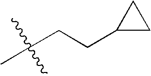 | CH₂CH₂NH₂ |
| 692 | 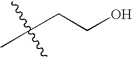 | 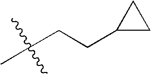 | CH₂CH₂NHMe |
| 693 | 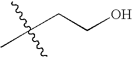 | 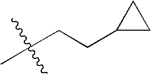 | CH₂CH₂NHEt |
| 694 | 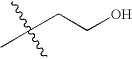 | 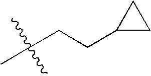 | CH₂CH₂NHPr |
| 695 | 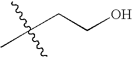 | 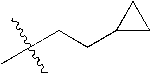 | CH₂CH₂NH(i-Pr) |
| 696 | 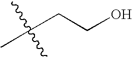 | 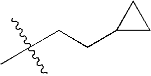 | CH₂CH₂NMe₂ |
| 697 | 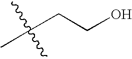 | 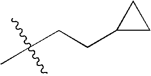 | CH₂CH₂NEt₂ |
| 698 | 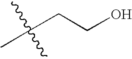 | 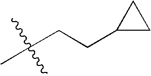 | CH₂CH₂N(n-Pr)₂ |
| 699 | 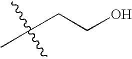 | 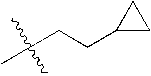 | 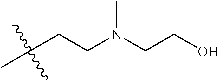 |
| 700 | 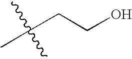 | 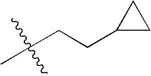 | 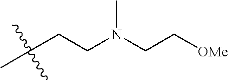 |
| 701 | 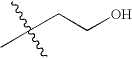 | 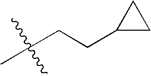 | 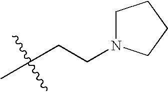 |
| 702 | 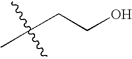 | 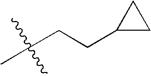 | 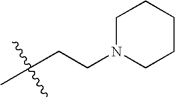 |
| 703 | 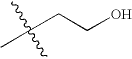 | 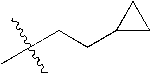 | 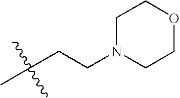 |
| 704 | 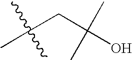 | Me | CH₂CH₂NH₂ |
| 705 | 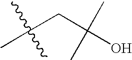 | Me | CH₂CH₂NHMe |
| 706 | 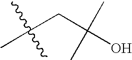 | Me | CH₂CH₂NHEt |
| 707 | 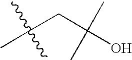 | Me | CH₂CH₂NHPr |
| 708 | 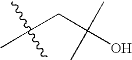 | Me | CH₂CH₂NH(i-Pr) |
| 709 | 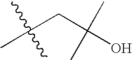 | Me | CH₂CH₂NMe₂ |
| 710 | 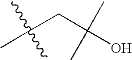 | Me | CH₂CH₂NEt₂ |
| 711 | 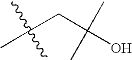 | Me | CH₂CH₂N(n-Pr)₂ |
| 712 | 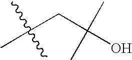 | Me | 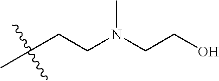 |
| 713 | 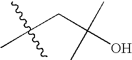 | Me | 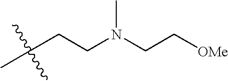 |
| 714 | 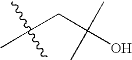 | Me | 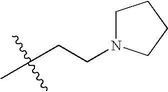 |
| 715 | 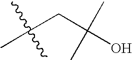 | Me | 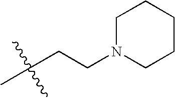 |
| 716 | 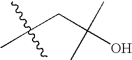 | Me | 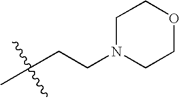 |
| 717 | 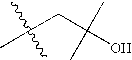 | Et | CH₂CH₂NH₂ |
| 718 | 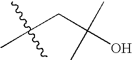 | Et | CH₂CH₂NHMe |
| 719 | 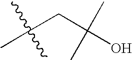 | Et | CH₂CH₂NHEt |
| 720 | 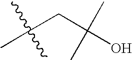 | Et | CH₂CH₂NHPr |
| 721 | 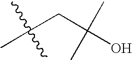 | Et | CH₂CH₂NH(i-Pr) |
| 722 | 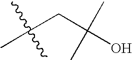 | Et | CH₂CH₂NMe₂ |
| 723 | 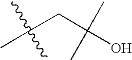 | Et | CH₂CH₂NEt₂ |
| 724 | 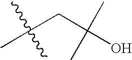 | Et | CH₂CH₂N(n-Pr)₂ |
| 725 | 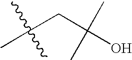 | Et | 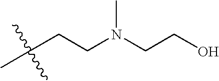 |
| 726 | 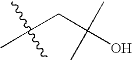 | Et | 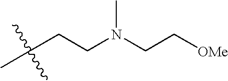 |
| 727 | 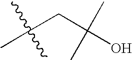 | Et | 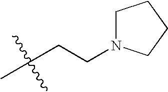 |
| 728 | 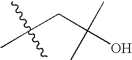 | Et | 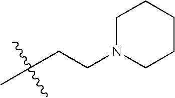 |
| 729 | 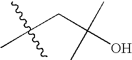 | Et | 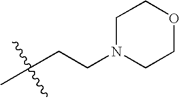 |
| 730 | 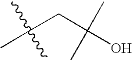 | 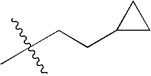 | CH₂CH₂NH₂ |
| 731 | 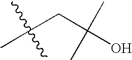 | 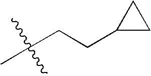 | CH₂CH₂NHMe |
| 732 | 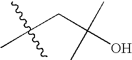 | 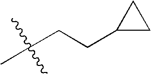 | CH₂CH₂NHEt |
| 733 | 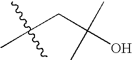 | 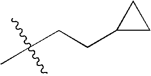 | CH₂CH₂NHPr |
| 734 | 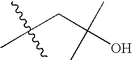 | 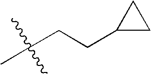 | CH₂CH₂NH(i-Pr) |
| 735 | 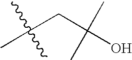 | 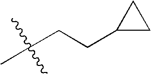 | CH₂CH₂NMe₂ |
| 736 | 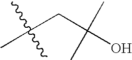 | 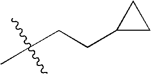 | CH₂CH₂NEt₂ |
| 737 | 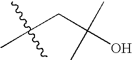 | 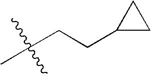 | CH₂CH₂N(n-Pr)₂ |
| 738 | 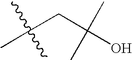 | 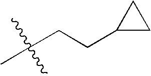 | 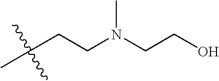 |
| 739 | 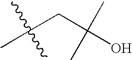 | 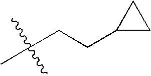 | 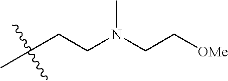 |
| 740 | 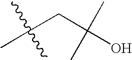 | 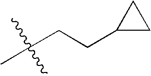 | 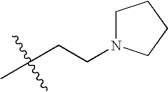 |
| 741 | 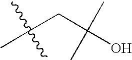 | 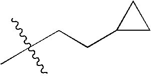 | 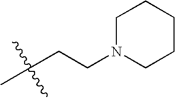 |
| 742 | 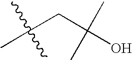 | 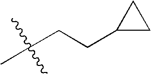 | 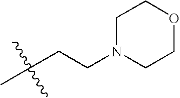 |
| 743 | 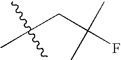 | Me | CH₂CH₂NH₂ |
| 744 | 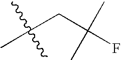 | Me | CH₂CH₂NHMe |
| 745 | 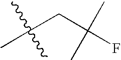 | Me | CH₂CH₂NHEt |
| 746 | 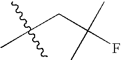 | Me | CH₂CH₂NHPr |
| 747 | 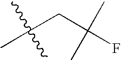 | Me | CH₂CH₂NH(i-Pr) |
| 748 | 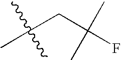 | Me | CH₂CH₂NMe₂ |
| 749 | 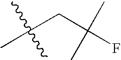 | Me | CH₂CH₂NEt₂ |
| 750 | 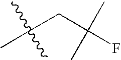 | Me | CH₂CH₂N(n-Pr)₂ |
| 751 | 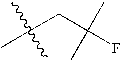 | Me | 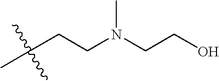 |
| 752 | 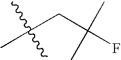 | Me | 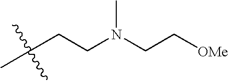 |
| 753 | 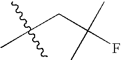 | Me | 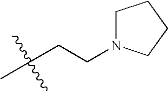 |
| 754 | 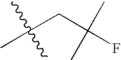 | Me | 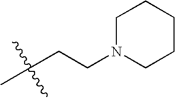 |
| 755 | 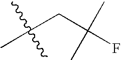 | Me | 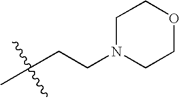 |
| 756 | 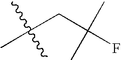 | Et | CH₂CH₂NH₂ |
| 757 | 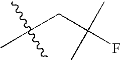 | Et | CH₂CH₂NHMe |
| 758 | 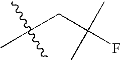 | Et | CH₂CH₂NHEt |
| 759 | 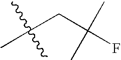 | Et | CH₂CH₂NHPr |
| 760 | 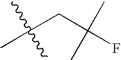 | Et | CH₂CH₂NH(i-Pr) |
| 761 | 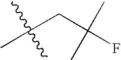 | Et | CH₂CH₂NMe₂ |
| 762 | 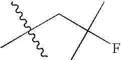 | Et | CH₂CH₂NEt₂ |
| 763 | 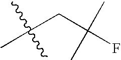 | Et | CH₂CH₂N(n-Pr)₂ |
| 764 | 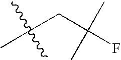 | Et | 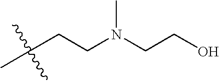 |
| 765 | 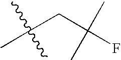 | Et | 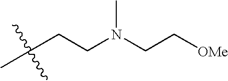 |
| 766 | 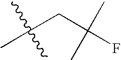 | Et | 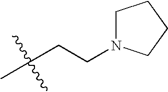 |
| 767 | 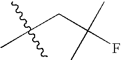 | Et | 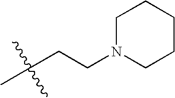 |
| 768 | 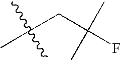 | Et | 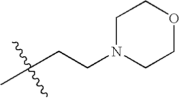 |
| 769 | 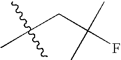 | 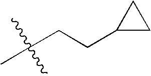 | CH₂CH₂NH₂ |
| 770 | 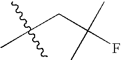 | 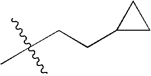 | CH₂CH₂NHMe |
| 771 | 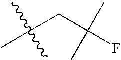 | 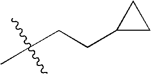 | CH₂CH₂NHEt |
| 772 | 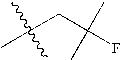 | 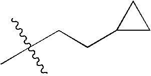 | CH₂CH₂NHPr |
| 773 | 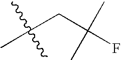 | 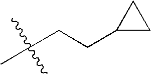 | CH₂CH₂NH(i-Pr) |
| 774 | 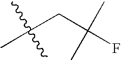 | 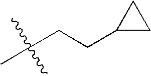 | CH₂CH₂NMe₂ |
| 775 | 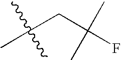 | 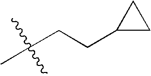 | CH₂CH₂NEt₂ |
| 776 | 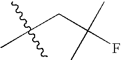 | 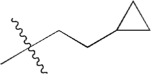 | CH₂CH₂N(n-Pr)₂ |
| 777 | 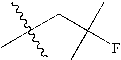 | 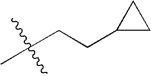 | 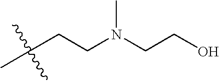 |
| 778 | 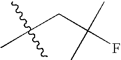 | 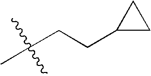 | 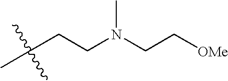 |
| 779 | 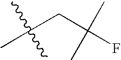 | 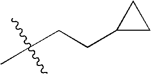 | 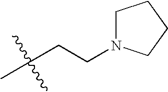 |
| 780 | 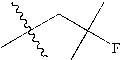 | 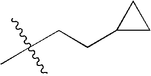 | 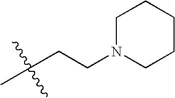 |
| 781 | 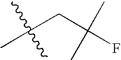 | 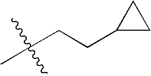 | 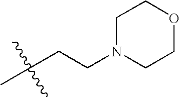 |

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A compound of Formula I: 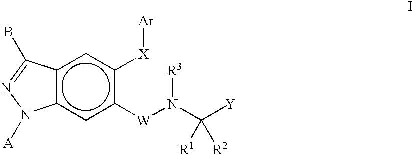
and metabolites, solvates, tautomers, resolved enantiomers, diastereomers, and salts thereof, wherein:
A is H, an amine protecting group, C₁-C₈ alkyl, C₂-C₈ alkenyl, C₂-C₈ alkynyl, saturated or partially unsaturated cycloalkyl, saturated or partially unsaturated heterocycloalkyl, aryl or heteroaryl, wherein said alkyl, alkenyl, alkynyl, cycloalkyl, heterocycloalkyl, aryl and heteroaryl are optionally substituted with one or more groups independently selected from F, Cl, Br, I, CN, ═O (with the proviso that it is not substituted on an aryl or heteroaryl), ═NOR⁴ (with the proviso that it is not substituted on an aryl or heteroaryl), —NR⁴R⁵, —NR⁴(C═O)R⁵, —NR⁴C(═O)NR⁵R⁷, —CR⁵═NOR⁴—SO₂R⁶, —SOR⁶, —SR⁵, —SO₂NR⁴R⁵, —OR⁴, —(C═O)R⁴, —(C═O)OR⁴, —O—(C═O)R⁴, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I;
B is H, —NH₂, —NHMe, —NMe₂, —CH₃, —CF₃, —CH₂OH, cyclopropyl, C₁-C₃ alkyl, —OH, CN, F, Cl, Br or I, wherein said alkyl is optionally substituted with one or more groups independently selected from F, Cl, Br and I;
W is C(═O) or SO₂;
X is O, S, SO, SO₂, NH, NCH₃, C(═O), CH₂, CH(CH₃), C(CH₃)₂, C═NOR⁴, C═CR⁴ or CHOR⁴;
Y is —C((═O)R⁴—C(═O)OR⁴, —C(═O)NR⁴R⁵, —CR⁴R⁵OR⁷, —C(═O)NR⁴OR⁵ or —C(═O)NR⁴NR⁵R⁷;
R¹ is C₁-C₈ alkyl, C₂-C₈ alkenyl, C₂-C₈ alkynyl, C₁-C₈ heteroalkyl, saturated or partially unsaturated cycloalkyl, or saturated or partially unsaturated heterocycloalkyl, wherein said alkyl, alkenyl, alkynyl, heteroalkyl, cycloalkyl and heterocycloalkyl are optionally substituted with one or more groups independently selected from F, Cl, Br, I, CN, ═O, ═NOR⁴, —NR⁴R⁵, —NR⁴(C═O)R⁵, —NR⁴C(═O)NR⁵R⁷, —CR⁵═NOR⁴—SO₂R⁶, —SOR⁶, —SR⁵, —SO₂NR⁴R⁵, —OR⁴, —(C═O)R⁴, —(C═O)OR⁴, —O—(C═O)R⁴, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I;
R² and R⁶ are independently alkyl, alkenyl, alkynyl, saturated or partially unsaturated cycloalkyl, saturated or partially unsaturated heterocycloalkyl, aryl or heteroaryl, wherein said alkyl, alkenyl, alkynyl, cycloalkyl, heterocycloalkyl, aryl and heteroaryl are optionally substituted with one or more groups independently selected from F, Cl, Br, I, CN, ═O (with the proviso that it is not substituted on an aryl or heteroaryl), ═NOR⁴ (with the proviso that it is not substituted on an aryl or heteroaryl), —NR⁴R⁵, —NR⁴(C═O)R⁵, —NR⁴C(═O)NR⁵R⁷, —CR⁵═NOR⁴—SO₂R⁶, —SOR⁶, —SR⁵, SO₂NR⁴R⁵, —OR⁴, —(C═O)R⁴, —(C═O)R⁴, —O—(C═O)R⁴, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br, I and OH,
or R¹ and R² together with the atom to which they are attached form a saturated or partially unsaturated 3-10 membered carbocyclic ring or a saturated or partially unsaturated 3-10 membered heterocyclic ring having 1 or more heteroatoms, wherein said carbocyclic and heterocyclic rings are optionally substituted with one or more groups independently selected from F, Cl, Br, I, CN, ═O, ═NOR⁴, —NR⁴R⁵, —NR⁴(C═O)R⁵, —NR⁴C(═O)NR⁵R⁷, —CR⁵═NOR⁴—SO₂R⁶, —SOR⁶, —SR⁵, SO₂NR⁴R⁵, —OR⁴, —(C═O)R⁴, —(C═O)OR⁴, —O—(C═O)R⁴, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I;
R³ is H, C₁-C₄ alkyl, C₃-C₆ cycloalkyl or —OR⁴, wherein said alkyl and cycloalkyl are optionally substituted with one or more groups independently selected from F, Cl, Br, I, and alkyl;
R⁴, R⁵ and R⁷ are independently H, alkyl, alkenyl, alkynyl, saturated or partially unsaturated cycloalkyl, saturated or partially unsaturated heterocycloalkyl, aryl, or heteroaryl, wherein said alkyl, alkenyl, alkynyl, cycloalkyl, heterocycloalkyl, aryl and heteroaryl are optionally substituted with one or more groups independently selected from F, Cl, Br, I, CN, ═O (with the proviso that it is not substituted on an aryl or heteroaryl), ═NOR⁸ (with the proviso that it is not substituted on an aryl or heteroaryl), —NR⁸R⁹, —NR⁸(C═O)R⁹, —NR⁸C(═O)NR⁹R¹⁰, —CR⁹═NOR⁸—SO₂R⁶, —SOR⁶, —SR⁹, —SO₂NR⁸R⁹, —OR⁸, —(C═O)R⁸, —(C═O)OR⁸, —O—(C═O)R⁸, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I,
or R⁴ and R⁵ together with the atom to which they are attached form a saturated or partially unsaturated 4-8 membered carbocyclic ring or a saturated or partially unsaturated 4-8 membered heterocyclic ring having 1 or more heteroatoms, wherein said carbocyclic and heterocyclic rings are optionally substituted with one or more groups independently selected from F, Cl, Br, I, CN, ═O, ═NOR⁸, —NR⁸R⁹, —NR⁸(C═O)R⁹, —NR⁸C(═O)NR⁹R¹⁰, —CR⁹═NOR⁸—SO₂R¹¹, —SOR¹¹, —SR⁹, SO₂NR⁸R⁹, —OR⁸, —(C═O)R⁸, —(C═O)OR⁸, —O—(C═O)R⁸, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I, and wherein said carbocyclic and heterocyclic rings are optionally fused to an aromatic ring,
or R⁵ and R⁷ together with the atom to which they are attached form a saturated or partially unsaturated 4-8 membered heterocyclic ring having 1 or more heteroatoms, wherein said heterocyclic ring is optionally substituted with one or more groups independently selected from F, Cl, Br, I, CN, ═O, ═NOR⁸, —NR⁸R⁹, —NR⁸(C═O)R⁹, —NR⁸C(═O)NR⁹R¹⁰, —CR⁹═NOR⁸—SO₂R¹¹, —SOR¹¹, —SR⁹, SO₂NR⁸R⁹, —OR⁸, —(C═O)R⁸, —(C═O)OR⁸, —O—(C═O)R⁸, alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I, and wherein said heterocyclic ring is optionally fused to an aromatic ring;
R⁸, R⁹ and R¹⁰ are independently H, alkyl, alkenyl, alkynyl, saturated or partially unsaturated cycloalkyl, saturated or partially unsaturated heterocycloalkyl, aryl, or heteroaryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br and I;
R¹¹ is alkyl, alkenyl, alkynyl, heteroalkyl, heteroalkenyl, heteroalkynyl, saturated or partially unsaturated cycloalkyl, saturated or partially unsaturated heterocycloalkyl, heteroaryl and aryl, wherein said alkyl, alkenyl, alkynyl, heteroalkyl, heteroalkenyl, heteroalkynyl, cycloalkyl, heterocycloalkyl, heteroaryl and aryl are optionally substituted with one or more groups independently selected from F, Cl, Br, and I; and
Ar is aryl or heteroaryl, wherein said aryl and heteroaryl are optionally substituted with one or more groups independently selected from —NH₂, —NHMe, —NMe₂, —CH₃, —CF₃, —CH₂OH, cyclopropyl, —OH, CN, F, Cl, Br, I, and C₁-C₃ alkyl, wherein said alkyl is optionally substituted with one or more groups independently selected from F, Cl, Br and I.

2. The compound of claim 1, wherein R¹ is C₁-C₈ alkyl optionally substituted with one or more groups independently selected from F, Cl, Br, I, alkyl and cycloalkyl.

3. The compound of claim 1, wherein R¹ is CH₃, CH₂CH₃, CH₂F, CHF₂, CF₃, CH₂-(cyclopropyl) or CH₂CH₂— (cyclopropyl).

4. The compound of claim 1, wherein R² is C₁-C₈ alkyl optionally substituted with —NR⁴R⁵, wherein R⁴ and R⁵ are independently H or C₁-C₈ alkyl group optionally substituted with one or more groups independently selected from OR⁸.

5. The compound of claim 1, wherein R² is CH₂CH₂NH₂, CH₂CH₂NHCH₃, CH₂CH₂NH(CH₂CH₃), CH₂CH₂NH(CH₂CH₂CH₃), CH₂CH₂NHCH(CH₃)₂, CH₂CH₂N(CH₃)₂, CH₂CH₂N(CH₂CH₃)₂, CH₂CH₂N(CH₂CH₂CH₃)₂, CH₂CH₂N(CH₃)CH₂CH₂OH, or CH₂CH₂N(CH₃)CH₂CH₂OCH₃.

6. The compound of claim 1, wherein R² is alkyl optionally substituted with NR⁴R⁵, wherein R⁴ and R⁵ together with the atoms to which they are attached form a 5 to 6 membered heterocyclic ring, and said heterocyclic ring is optionally substituted with one or more groups independently selected from OR⁸.

7. The compound of claim 1, wherein R² is selected from the structures:

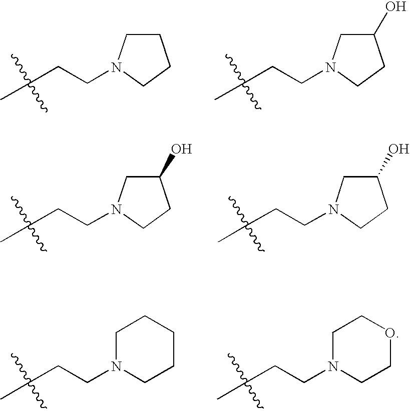

8. The compound of claim 1, wherein Y is —C(═O)OR⁴ or —C(═O)NR⁴R⁵.

9. The compound of claim 1, wherein Y is —C(═O)NR⁴R⁵ and R⁴ and R⁵ are independently H or an alkyl group optionally substituted with one or more groups independently selected from OR⁸.

10. The compound of claim 1, wherein Y is —C(═O)NH₂, —C(═O)NHCH₃, —C(═O)N(CH₃)₂, and —C(═O)NH(CH₂CH₂OH).

11. The compound of claim 1, wherein Y is —C(═O)NR⁴R⁵ and R⁴ and R⁵ together with the atoms to which they are attached form a 5 to 6 membered heterocyclic ring.

12. The compound of claim 1, wherein Y is selected from the structures

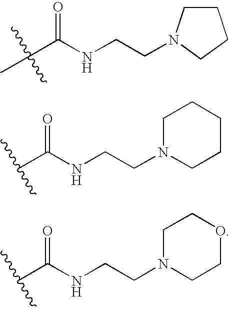

13. The compound of claim 1, wherein Y is —C(═O)OR⁴.

14. The compound of claim 1, wherein Y is —C(═O)OH, —C(═O)OCH₃, or —C(═O)OCH₂CH₃.

15. The compound of claim 1, wherein A is H, C₁-C₈ alkyl or C₂-C₈ alkenyl, wherein said alkyl and alkenyl are optionally substituted with one or more groups independently selected from F, Cl, Br, I, alkyl and OR⁴.

16. The compound of claim 1, wherein A is methyl, isopropyl, CH₂C(CH₃)₂F, CH₂CH₂OH, or CH₂C(CH₃)₂OH.

17. The compound of claim 1, wherein Ar is phenyl optionally substituted by one or more groups independently selected from —NH₂, —NHMe, —NMe₂, —CH₃, —CF₃, —CH₂OH, cyclopropyl, C₁-C₃ alkyl, —OH, CN, F, Cl, Br and I, wherein said alkyl is optionally substituted with one or more groups independently selected from F, Cl, Br and I.

18. The compound of claim 1, wherein Ar is phenyl, 2-chlorophenyl, 3-chlorophenyl, 4-chlorophenyl, 2-fluorophenyl, 3-fluorophenyl, 4-fluorophenyl, 2,4-difluorophenyl, 2,5-difluorophenyl, 2-methylphenyl, 3-methylphenyl, 4-methylphenyl, 2-methoxyphenyl, 3-methoxyphenyl, 4-methoxyphenyl, 2-trifluoromethylphenyl, 3-trifluoromethylphenyl, 4-trifluoromethylphenyl, 2-trifluoromethoxyphenyl, 3-trifluoromethoxyphenyl, 4-trifluoromethoxyphenyl, and 4-(3,5-bis-trifluoromethylphenyl).

19. The compound of claim 1, wherein Ar is 2,4-difluorophenyl.

20. The compound of claim 1, wherein X is O.

21. The compound of claim 1, wherein W is C═O.

22. The compound of claim 1, wherein B is H.

23. The compound of claim 1, wherein R³ is H.

24. The compound of claim 1, wherein said compound of Formula I has the configuration:

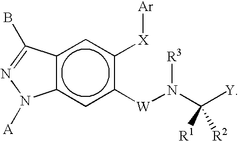

25. The compound of claim 1, selected from
(S)-Methyl 2-(5-(2,4-difluorophenoxy)-1-isobutyl-1H-indazole-6-carboxamido)-4-(dimethylamino)-2-methylbutanoate;
(S)-methyl 2-(cyclopropylmethyl)-2-(5-(2,4-difluorophenoxy)-1-isobutyl-1H-indazole-6-carboxamido)-4-(dimethylamino)butanoate;
(S)-Methyl 2-(5-(2,4-difluorophenoxy)-1-isobutyl-1H-indazole-6-carboxamido)-2-methyl-4-(pyrrolidin-1-yl)butanoate;
(S)-N-(1-amino-4-(dimethylamino)-2-methyl-1-oxobutan-2-yl)-5-(2,4-difluorophenoxy)-1-isobutyl-1H-indazole-6-carboxamide;
(S)-N-(1-amino-2-methyl-1-oxo-4-(pyrrolidin-1-yl)butan-2-yl)-5-(2,4-difluorophenoxy)-1-isobutyl-1H-indazole-6-carboxamide; and
(S)-5-(2,4-difluorophenoxy)-N-(4-(dimethylamino)-1-hydroxy-2-methylbutan-2-yl)-1-isobutyl-1H-indazole-6-carboxamide, and salts thereof.

26. A method of treating a p38-mediated condition in a human or animal, comprising administering to said human or animal a compound of claim 1, or a pharmaceutically acceptable salt or prodrug thereof in an amount effective to treat or prevent said p38-mediated condition.

27. The method of claim 26 wherein said p38-mediated condition is inflammatory disease, autoimmune disease, destructive bone disorder, hyperproliferative disorder, infectious disease, viral disease, or neurodegenerative disease.

28. A compound of claim 1 for use in therapy.

29. The use of a compound of claim 1 in the manufacture of a medicament for use as a p38 inhibitor.

30. A composition comprising a compound of claim 1.

31. A kit for treating a p38-mediated condition, wherein said kit comprises:
a) a first pharmaceutical composition comprising a compound of claim 1 or a pharmaceutically acceptable salt or prodrug thereof; and
b) instructions for use.

32. A method of preparing a compound of claim 1, comprising:
reacting a compound having the formula 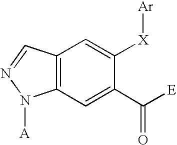
wherein E is halogen or 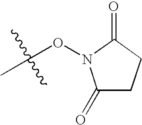
with an amino compound having the formula 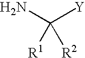
wherein Y is C(═O)NH₂ or C(═O)OMe, in the presence of a base.

33. The method of claim 32, wherein said amino compound has the formula

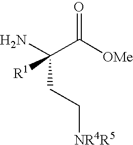

34. The method of claim 33, wherein said amino compound is prepared by the method comprising:
treating a compound having the formula 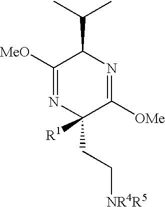
with an acid.
